(19) United States
(12) Patent Application Publication
(10) Pub. No.: US 2010/0292279 A1
(43) Pub. Date: Nov. 18, 2010

(54) Bis-(Sulfonylamino) Derivatives in Therapy

(75) Inventors: Johan Bylund, Sodertalje (SE); Maria E. Ek, Sodertalje (SE); Jörg Holenz, Sodertalje (SE); Annika Kers, Sodertalje (SE); Liselotte Öhberg, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/778,355

(22) Filed: May 12, 2010

Related U.S. Application Data
(60) Provisional application No. 61/178,104, filed on May 14, 2009.

(51) Int. Cl. A61K 31/44; C07C 311/16; A61K 31/18; C07D 307/78; A61K 31/343; C07D 207/04; A61K 31/40; C07D 213/81; A61P 29/00; A61P 25/02; A61P 19/02; A61P 35/00; A61P 9/10; A61P 25/00; A61P 25/28

(52) U.S. Cl. 514/354; 564/83; 514/603; 549/469; 514/469; 548/578; 514/408; 546/323

(57) ABSTRACT

The invention provides compounds of formula

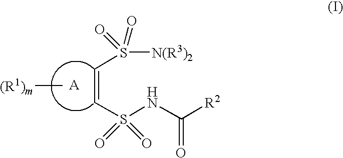

wherein R¹, R², R³, A and m are as defined in the specification and optical isomers, racemates and tautomers thereof, and pharmaceutically acceptable salts thereof; together with processes for their preparation, pharmaceutical compositions containing them and their use in therapy. The compounds are inhibitors of microsomal prostaglandin E synthase-1.

### FIELD OF THE INVENTION

The present invention relates to bis-(sulfonylamino) derivatives, processes for their preparation, pharmaceutical compositions containing them and their use in therapy.

### BACKGROUND OF THE INVENTION

Modulation of prostaglandin metabolism is at the center of current anti-inflammatory therapies. NSAIDs and COX-2 inhibitors block the activity of cyclooxygenases and their ability to convert arachidonic acid into prostaglandin H2 (PGH2). PGH2 can be subsequently metabolized by terminal prostaglandin synthases to the corresponding biologically active PGs, namely, PGI2, thromboxane (Tx) A2, PGD2, PGF2α, and PGE2. A combination of pharmacological, genetic and neutralizing antibody approaches demonstrates the importance of PGE2 in inflammation. The conversion of PGH2 to PGE2 is by prostaglandin E synthases (PGES) may therefore represent a pivotal step in the propagation of inflammatory stimuli.

Microsomal prostaglandin E synthase-1 (mPGES-1) is an inducible PGES after exposure to pro-inflammatory stimuli. mPGES-1 is induced in the periphery and in the CNS by inflammation and represents therefore a target for acute and chronic inflammatory disorders.

PGE2 is a major prostanoid driving inflammatory processes. The Prostanoid is produced from arachidonic acid liberated by Phospholipases (PLAs). Arachidonic acid is tranformed by the action of Prostaglandin H Synthase (PGH Synthase, cycloxygenase) into PGH2 which is a substrate for mPGES-1, that is the terminal enzyme transforming PGH2 to the pro-inflammatory PGE2.

NSAIDs reduce PGE2 by inhibiting cyclooxygenase, but at the same time reducing other prostanoids, giving side effects such as ulcerations in the GI tract. mPGES-1 inhibition gives a similar effect on PGE2 production without affecteing the formation of other prostanoids, and hence a more favourable profile.

By blocking the formation of PGE2 in animal models of inflammatory pain a reduced inflammation, pain and fever response has been demonstrated, Kojima et. al, The Journal of Immunology 2008, 180, 8361-6, Xu et. al., The Journal of Pharmacology and Experimental Therapeutics 2008, 326, 754-63.

In abdominal aortic aneurism, inflammation leads to connective tissue degradation and smooth muscle apoptosis ultimately leading to aortic dilation and rupture. In animals lacking mPGES-1a slower disease progression and disease severity has been demonstrated Wang et. al. Circulation, 2008, 117, 1302-1309.

Several lines of evidence indicate that PGE2 is involved in malignant growth. PGE2 facilitates tumour progression by stimulation of cellular proliferation and angiogenesis and by modulation of immunosupression. In support of a role for PGE2 in carcinogenesis genetic deletion of mPGES-1 in mice supresses the intestinal tumourogenesis (Nakanishi et. al. Cancer Research 2008, 68(9), 3251-9) and reduces tumour growth in a lung xenograft model (Kamei et al., Biochem J 2010 425(2):361-71). In man, mPGES-1 is also upregulated in cancers such as colorectal cancer (Schröder Journal of Lipid Research 2006, 47, 1071-80) and in NSCLC, Non Small Cell Lung Carcinoma (Yoshimatsu et al Clinical Cancer Research 2001, 7(9): 2669-74). Furthermore, in lung tumours levels of PGE2 are also elevated (McLemore et al., Cancer Research 1988 48(11):3140-7) and high expression of COX2 correlates with poor prognosis (Mascaux Br J Cancer. 2006 Jul. 17; 95(2):139-45).

Myositis is chronic muscle disorder characterized by muscle weakness and fatigue. Proinflammatory cytokines and prostanoids have been implicated in the development of myositis. In skeletal muscle tissue from patients suffering from myositis an increase in cyclooxygenases and mPGES-1 has been demonstrated, implicating mPGES-1 as a target for treating this condition. Korotkova Annals of the Rheumatic Diseases 2008, 67, 1596-1602.

In atherosclerosis inflammation of the vasculature leads to atheroma formation that eventually may progress into infarction. In patients with carotid atherosclerosis an increase in mPGES-1 in plauqe regions have been found Gómez-Hernández Atherosclerosis 2006, 187, 139-49. In an animal model of atherosclerosis, mice lacking the mPGES-1 receptor was found to show a retarded atherogenesis and a concommitant reduction in macrophage-derived foam cells together with an increase in vascular smooth muscle cells. Wang Proceedings of National Academy of Sciences 2006, 103(39), 14507-12.

The present invention is directed to novel compounds that are selective inhibitors of the microsomal prostaglandin E synthase-1 enzyme and would therefore be useful for the treatment of pain and inflammation in a variety of diseases or conditions.

### DISCLOSURE OF THE INVENTION

In one aspect we disclose a compound of formula (I) or a pharmaceutically acceptable salt thereof

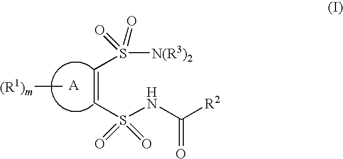

wherein:
A is selected from mono- and bicyclic aryl, mono- and bicyclic heteroaryl, cycloalkenyl and mono- and bicyclic heterocyclyl;
R¹ is independently selected from halogen, nitro, SF₅, CHO, C₀₋₆alkylCN, OC₁₋₆alkylCN, CO₀₋₆alkylOR⁵, OC₂₋₆alkylOR⁵, C₀₋₆alkylNR⁵R⁶, OC₂₋₆alkylNR⁵R⁶, OC₂₋₆alkylOC₂₋₆alkylNR⁵R⁶, C₀₋₆alkylCO₂R⁵, OC₁₋₆alkylCO₂R⁵, C₀₋₆alkylCON(R⁵)₂, OC₁₋₆alkylCON(R⁵)₂, OC₂₋₆alkylNR⁵(CO)R⁶, C₀₋₆alkylNR⁵(CO)R⁶, O(CO)NR⁵R⁶, NR⁵(CO)OR⁶, NR⁵(CO)NR⁵R⁶, O(CO)OR⁵, O(CO)R⁵, C₀₋₆alkylCOR⁵, OC₁₋₆alkylCOR⁵, NR⁵(CO)(CO)R⁵, NR⁵(CO)(CO)NR⁵R⁶, C₀₋₆alkylSR⁵, C₀₋₆alkyl(SO₂)NR⁵R⁶, OC₁₋₆alkylNR⁵(SO₂)R⁶, OC₀₋₆alkyl(SO₂)NR⁵R⁶, C₀₋₆alkyl(SO)NR⁵R⁶, OC₁₋₆alkyl(SO)NR⁵R⁶, C₀₋₆alkylOSO₂R⁵, C₀₋₆alkylNR⁵(SO₂)NR⁵R⁶, C₀₋₆alkylNR⁵(SO)R⁶, OC₂₋₆alkylNR⁵(SO)R⁶, OC₁₋₆alkylSO₂R⁵, C₀₋₆alkylSO₂R⁵, C₀₋₆alkylSOR⁵, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, CO₀₋₆alkylC₃₋₈cycloalkyl, C₀₋₆alkylaryl, C₀₋₆alkylheteroaryl or C₀₋₆alkylheterocyclyl is optionally substituted with one or more B, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with one or more B;

### R² is -L¹-G¹-L²-G²;

R³ is hydrogen;
G¹ is selected from C₃₋₁₀cycloalkyl, C₄₋₁₂cycloalkenyl, C₂₋₁₂cycloalkynyl, aryl, heteroaryl, heterocyclyl, wherein said C₃₋₁₀cycloalkyl, C₄₋₁₂cycloalkenyl, C₂₋₁₂cycloalkynyl, aryl, heteroaryl or heterocyclyl is optionally substituted with one or more R¹⁰;
G² is selected from hydrogen, C₃₋₈cycloalkyl, C₄₋₁₂cycloalkenyl, C₇₋₁₂cycloalkynyl, aryl, heteroaryl, heterocyclyl, wherein said C₃₋₈cycloalkyl, C₄₋₁₂cycloalkenyl, C₇₋₁₂cycloalkynyl, aryl, heteroaryl or heterocyclyl is optionally substituted with one or more R¹⁰;
At each occurrence, R⁵ is independently selected from hydrogen, C₁₋₆alkyl, C₂₋₆alkenyl, C₂₋₆alkynyl, CO₀₋₆alkylC₃₋₈cycloalkyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl or C₀₋₆alkylheterocyclyl is optionally substituted with one or more B;
At each occurrence, R⁶ is selected from hydrogen, C₁₋₆alkyl, C₂₋₆alkenyl, C₂₋₆alkynyl, C₂₋₆alkylOR⁵, C₀₋₆alkylC₃₋₈cycloalkyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl or C₀₋₆alkylheterocyclyl is optionally substituted with one or more B; or
R⁵ and R⁶ may together with the linking atom or atoms to which they are bonded form a 4 to 6 membered heterocyclic ring containing one or more heteroatoms selected from N, O or S that is optionally substituted with B; whenever two R⁵ groups occur in the structure then they may optionally together with the linking atom or atoms to which they are bonded form a 5 or 6 membered heterocyclic ring containing one or more heteroatoms selected from N, O or S, that is optionally substituted with one or more B;
L¹ and L² independently represent a bond or a 1-7 membered non-cyclic linking group containing 0-2 heteroatoms selected from O, N, and S, said linking group optionally containing CO, S(O)ₙ, C═C or an acetylenic group, and optionally being substituted with one or more R⁸;
R⁸ is selected from halogen, nitro, CHO, CN, OH, OC₁₋₆alkyl, O(C₁₋₆alkyl)O(C₁₋₆alkyl), C₁₋₆alkyl, C₂₋₆alkenyl, C₂₋₆alkynyl N(C₁₋₆alkyl)(C₁₋₆alkyl), NH₂, NH(C₁₋₆alkyl), S(O)ₙ(C₁₋₆alkyl), SO₂N(C₁₋₆alkyl)(C₁₋₆alkyl), SO₂NH₂, SO₂NH(C₁₋₆alkyl), CF₃, CHF₂, CFH₂, C(O)(C₁₋₆alkyl), C(O)N(C₁₋₆alkyl)(C₁₋₆alkyl), C(O)NH(C₁₋₆alkyl), C(O)NH₂, N(C₁₋₆alkyl)(CO)N(C₁₋₆alkyl)(C₁₋₆alkyl), NH(CO)N(C₁₋₆alkyl)(C₁₋₆alkyl), N(C₆₋₆alkyl)(CO)NH(C₁₋₆alkyl), NH(CO)NH₂, N(C₁₋₆alkyl)(CO)NH₂;
Whenever two R⁸ groups are connected to the same atom of the linking group L¹, they may optionally together form a 3 to 6 membered non-aromatic, carbocyclic or heterocyclic (containing one or more heteroatoms selected from N, O or S) ring, that is optionally substituted with one or more R⁹;
R⁹ is selected from halogen, nitro, CHO, CN, OH, OC₁₋₆alkyl, O(C₁₋₆alkyl)O(C₁₋₆alkyl), C₁₋₆alkyl, C₂₋₆alkenyl, C₂₋₆alkynyl N(C₁₋₆alkyl)(C₁₋₆alkyl), NH₂, NH(C₁₋₆alkyl), S(O)ₙ(C₁₋₆alkyl), SO₂N(C₁₋₆alkyl)(C₁₋₆alkyl), SO₂NH₂, SO₂NH(C₁₋₆alkyl), CF₃, CHF₂, CFH₂, C(O)(C₁₋₆alkyl), C(O)N(C₁₋₆alkyl)(C₁₋₆alkyl), C(O)NH(C₁₋₆alkyl), C(O)NH₂, N(C₁₋₆alkyl)(CO)N(C₁₋₆alkyl)(C₁₋₆alkyl), NH(CO)N(C₁₋₆alkyl)(C₁₋₆alkyl), N(C₁₋₆alkyl)(CO)NH(C₁₋₆alkyl), NH(CO)NH₂, N(C₁₋₆alkyl)(CO)NH₂;
B is selected from halogen, nitro, SF₅, OSF₅, CN, OR¹⁵, OC₂₋₆alkylNR¹⁵R¹⁶, NR¹⁵R¹⁶, CONR¹⁵R¹⁶, NR¹⁵(CO)R¹⁶, O(CO)C₁₋₆alkyl, (CO)OC₁₋₆alkyl, COR¹⁵, (SO₂)NR¹⁵R¹⁶, NR¹⁵SO₂R¹⁵, SO₂R¹⁵, SOR¹⁵, (CO)C₁₋₆alkylNR¹⁵R¹⁶, (SO₂)C₁₋₆alkylNR¹⁵R¹⁶, OSO₂R¹⁵, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
R¹⁵ is selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
R¹⁶ is selected from hydrogen, C₁₋₆alkyl, C₂₋₆alkenyl, C₂₋₆alkynyl, C₀₋₆alkylOR⁵, C₀₋₆alkylC₃₋₈cycloalkyl, C₀₋₆alkylaryl, C₀₋₆alkylheteroaryl and C₀₋₆alkylheterocyclyl; or
R¹⁵ and R¹⁶ may together with the linking atom or atoms to which they are bonded form a 4 to 6 membered heterocyclic ring containing one or more heteroatoms selected from N, O or S; whenever two R¹⁵ groups occur in the structure then they may optionally together with the linking atom or atoms to which they are bonded form a 5 or 6 membered heterocyclic ring containing one or more heteroatoms selected from N, O or S;
D is selected from halogen, nitro, SF₅, OSF₅, CN, OR¹³, OC₂₋₆alkylNR¹³R¹⁴, NR¹³R¹⁴, CONR¹³R¹⁴, NR¹³(CO)R¹⁴, O(CO)C₁₋₆alkyl, (CO)OC₁₋₆alkyl, COR¹³, (SO₂)NR¹³R¹⁴, NR¹³SO₂R¹⁴, SO₂R¹³, SOR¹³, (CO)C₁₋₆alkylNR¹³R¹⁴, (SO₂)C₁₋₆alkylNR¹³R¹⁴, OSO₂R¹³, C₁₋₆alkyl, C₂₋₆alkenyl, C₂₋₆alkynyl, C₀₋₆alkylC₃₋₈cycloalkyl, and C₀₋₆alkylheterocyclyl;
R¹⁰ is independently selected from halogen, nitro, SF₅, OSF₅, CN, OR¹¹, C≡CR¹¹, OC₂₋₆alkylNR¹¹R¹², NR¹¹R¹², CONR¹¹R¹², NR¹¹(CO)R¹², O(CO)C₁₋₆alkyl, (CO)OC₁₋₆alkyl, COR¹¹, (SO₂)NR¹¹R¹², NR¹¹SO₂R¹¹, SO₂R¹¹, SOR¹¹, (CO)C₁₋₆alkylNR¹¹R¹², (SO₂)C₁₋₆alkylNR¹¹R¹², OSO₂R¹¹, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl, C₀₋₆alkylheterocyclyl and OC₂₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl, C₀₋₆alkylheterocyclyl or OC₂₋₆alkylheterocyclyl is optionally substituted with one or more E, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with one or more E;

At each occurrence, R¹¹ is independently selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein any of the individual C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl groups may be optionally substituted with one or more E;

R¹² is selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein any of the individual C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl groups may be optionally substituted with one or more E; or
R¹¹ and R¹² may together with the linking atom or atoms to which they are bonded form a 4 to 6 membered heterocyclic ring containing one or more heteroatoms selected from N, O or S that is optionally substituted with B; whenever two R¹¹ groups occur in the structure then they may optionally together with the linking atom or atoms to which they are bonded form a 5 or 6 membered heterocyclic ring containing one or more heteroatoms selected from N, O or S, where the ring system is optionally substituted with one or more E;
R¹³ is independently selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
R¹⁴ is selected from hydrogen, C₁₋₆alkyl, C₂₋₆alkenyl, C₂₋₆alkynyl, C₀₋₆alkylOR⁵, C₀₋₆alkylC₃₋₈cycloalkyl, C₀₋₆alkylaryl, C₀₋₆alkylheteroaryl and C₀₋₆alkylheterocyclyl; or
R¹³ and R¹⁴ may together with the linking atom or atoms to which they are bonded form a 4 to 6 membered heterocyclic ring containing one or more heteroatoms selected from N, O or S; whenever two R¹³ groups occur in the structure then they may optionally together with the linking atom or atoms to which they are bonded form a 5 or 6 membered heterocyclic ring containing one or more heteroatoms selected from N, O or S;
E is selected from halogen, nitro, SF₅, OSF₅, CN, OR⁵, OC₂₋₆alkylNR⁵R⁶, NR⁵R⁶, CONR⁵R⁶, NR⁵(CO)R⁶, O(CO)C₁₋₆alkyl, (CO)OC₁₋₆alkyl, COR^{S}, (SO₂)NR⁵R⁶, NR⁵SO₂R⁵, SO₂R⁵, SOR⁵, (CO)C₁₋₆alkylNR⁵R⁶, (SO₂)C₁₋₆alkylNR⁵R⁶, OSO₂R⁵, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and CO₀₋₆alkylheterocyclyl;
m=0, 1, 2, 3, 4;
n=0, 1, 2;
wherein said compound is selected from the group consisting of:
4-(Benzyloxy)-3-(cyclopropylmethoxy)-N-(2-sulfamoylphenylsulfonyl)benzamide;
3-Methoxy-N-(2-sulfamoylphenylsulfonyl)-4-(p-tolyloxymethyl)benzamide;
3-(2,2-Difluoroethoxy)-4-(phenylethynyl)-N-(2-sulfamoylphenylsulfonyl)benzamide;
3-(2,2-Difluoroethoxy)-N-(2-sulfamoylphenylsulfonyl)-4-(p-tolylethynyl)benzamide;
4-(Cyclopentylethynyl)-3-(2,2-difluoroethoxy)-N-(2-sulfamoylphenylsulfonyl)benzamide;
3-(2,2-Difluoroethoxy)-4-(3,3-dimethylbut-1-ynyl)-N-(2-sulfamoylphenylsulfonyl)-benzamide;
4-(Benzofuran-2-yl)-3-(2,2-difluoroethoxy)-N-(2-sulfamoylphenylsulfonyl)benzamide;
N-(2-Sulfamoylphenylsulfonyl)-4-(o-tolylethynyl)benzamide;
N-(2-sulfamoylphenylsulfonyl)-4-(p-tolylethynyl)benzamide;
4-((4-Chlorophenyl)ethynyl)-N-(2-sulfamoylphenylsulfonyl)benzamide;
4-((4-Fluorophenyl)ethynyl)-N-(2-sulfamoylphenylsulfonyl)benzamide;
4-((3-Chlorophenyl)ethynyl)-N-(2-sulfamoylphenylsulfonyl)benzamide;
4-((3-Fluorophenyl)ethynyl)-N-(2-sulfamoylphenylsulfonyl)benzamide;
3-Methoxy-N-(2-sulfamoylphenylsulfonyl)-4-(o-tolylethynyl)benzamide;
4-((4-Chlorophenyl)ethynyl)-3-methoxy-N-(2-sulfamoylphenylsulfonyl)benzamide;
4-((4-Fluorophenyl)ethynyl)-3-methoxy-N-(2-sulfamoylphenylsulfonyl)benzamide;
3-Methoxy-N-(2-sulfamoylphenylsulfonyl)-4-(p-tolylethynyl)benzamide;
3-Methoxy-N-(2-sulfamoylphenylsulfonyl)-4-(m-tolylethynyl)benzamide;
4-((3-Chlorophenyl)ethynyl)-3-methoxy-N-(2-sulfamoylphenylsulfonyl)benzamide;
4-((3-Fluorophenyl)ethynyl)-3-methoxy-N-(2-sulfamoylphenylsulfonyl)benzamide;
4-(Benzofuran-2-yl)-3-(cyclopropylmethoxy)-N-(2-sulfamoylphenylsulfonyl)benzamide;
3-(Cyclopropylmethoxy)-4-(3,3-dimethylbut-1-ynyl)-N-(2-sulfamoylphenylsulfonyl)-benzamide;
3-(Cyclopropylmethoxy)-4-(3-methylbut-1-ynyl)-N-(2-sulfamoylphenylsulfonyl)-benzamide;
N-(2-Sulfamoylphenylsulfonyl)-4-((3,3,4,4-tetrafluoropyrrolidin-1-yl)methyl)benzamide;
3-(3-Hydroxy-3-methylbutoxy)-4-(3-methylbut-1-ynyl)-N-(2-sulfamoylphenylsulfonyl)-benzamide;
3-Isopropoxy-4-(3-methylbut-1-ynyl)-N-(2-sulfamoylphenylsulfonyl)benzamide;
4-(Cyclopropylethynyl)-3-(3-methoxy-3-methylbutoxy)-N-(2-sulfamoylphenylsulfonyl)-benzamide;
3-Isobutoxy-4-(3-methylbut-1-ynyl)-N-(2-sulfamoylphenylsulfonyl)benzamide;
3-Methoxy-4-(4-methylbenzyloxy)-N-(2-sulfamoylphenylsulfonyl)benzamide;
4-(Benzyloxy)-N-(2-sulfamoylphenylsulfonyl)-3-(3,3,3-trifluoropropoxy)benzamide;
4-(3,3-Dicyclopropyl-3-hydroxyprop-1-ynyl)-N-(2-sulfamoylphenylsulfonyl)-benzamide;
3-(Hydroxymethyl)-4-(3-methylhex-1-ynyl)-N-(2-sulfamoylphenylsulfonyl)benzamide;
3-Isopropoxy-N-(2-sulfamoylphenylsulfonyl)-4-((4-(trifluoromethyl)phenyl)ethynyl)-benzamide;
4-(3-Methylhex-1-ynyl)-N-(2-sulfamoylphenylsulfonyl)-3-(2-(2,2,2-trifluoroethoxy)-ethoxy)benzamide;
4-(3-Methylhex-1-ynyl)-N-(2-sulfamoylphenylsulfonyl)-3-(3,3,3-trifluoropropoxy)benzamide;
4-(3,3-Dicyclopropylprop-1-ynyl)-N-(2-sulfamoylphenylsulfonyl)-3-(3,3,3-trifluoropropoxy)benzamide;
3-Methoxy-4-(3-methylhex-1-ynyl)-N-(2-sulfamoylphenylsulfonyl)benzamide;
3-Methoxy-N-(2-sulfamoylphenylsulfonyl)-4-((4-(trifluoromethyl)phenyl)ethynyl)-benzamide;
3-(Hydroxymethyl)-N-(2-sulfamoylphenylsulfonyl)-4-((4-(trifluoromethyl)phenyl)-ethynyl)benzamide;
4-(3-Methylhex-1-ynyl)-N-(2-sulfamoylphenylsulfonyl)benzamide;
N-(2-Sulfamoylphenylsulfonyl)-4-((4-(trifluoromethyl)phenyl)ethynyl)benzamide;
4-(6-Chlorohex-1-ynyl)-N-(2-sulfamoylphenylsulfonyl)benzamide;
N-(2-Sulfamoylphenylsulfonyl)-6-((4-(trifluoromethyl)phenyl)ethynyl)nicotinamide;
5-Chloro-6-(3-methylbut-1-ynyl)-N-(2-sulfamoylphenylsulfonyl)nicotinamide;
4-(3-Methylbut-1-ynyl)-N-(2-sulfamoylphenylsulfonyl)-3-(3,3,3-trifluoropropoxy)-benzamide;
5-Fluoro-6-(3-methylbut-1-ynyl)-N-(2-sulfamoylphenylsulfonyl)nicotinamide;
5-Fluoro-N-(2-sulfamoylphenylsulfonyl)-6-((4-(trifluoromethyl)phenyl)ethynyl)-nicotinamide;
N-(2-Sulfamoylphenylsulfonyl)-6-((4-(trifluoromethyl)phenyl)ethynyl)-5-(3,3,3-trifluoro-propoxy)nicotinamide;
6-(3-Methylbut-1-ynyl)-N-(2-sulfamoylphenylsulfonyl)-5-(3,3,3-trifluoropropoxy)-nicotinamide;
3-Methyl-4-(3-methylbut-1-ynyl)-N-(2-sulfamoylphenylsulfonyl)benzamide;
4-(3-Methylbut-1-ynyl)-N-(2-sulfamoylphenylsulfonyl)-3-(2-(2,2,2-trifluoroethoxy)-ethoxy)benzamide;
3-(2,2-Difluoroethoxy)-4-(3-methylbut-1-ynyl)-N-(2-sulfamoylphenylsulfonyl)-benzamide;
4-(3-Methylbut-1-ynyl)-N-(2-sulfamoylphenylsulfonyl)-3-(2,2,2-trifluoroethoxy)-benzamide;
4-(3-Methylbut-1-ynyl)-N-(2-sulfamoylphenylsulfonyl)-3-(trifluoromethoxy)benzamide;
4-(3-Methylbut-1-ynyl)-N-(2-sulfamoylphenylsulfonyl)-3-((3,3,3-trifluoropropoxy)-methyl)benzamide;
4-(3-Methylbut-1-ynyl)-N-(2-sulfamoylphenylsulfonyl)-3-((2,2,2-trifluoroethoxy)methyl)-benzamide;
4-(Benzofuran-2-yl)-3-fluoro-N-(2-sulfamoylphenylsulfonyl)benzamide;
4-(3-Methylbut-1-ynyl)-N-(2-sulfamoylphenylsulfonyl)-3-(2,2,3,3-tetrafluoropropoxy)-benzamide;
4-(Benzofuran-2-yl)-N-(2-sulfamoylphenylsulfonyl)-3-(3,3,3-trifluoropropoxy)benzamide;
4-(3,3-Dimethylbut-1-ynyl)-N-(2-sulfamoylphenylsulfonyl)-3-(3,3,3-trifluoropropoxy)-benzamide;
4-(3,3-Dimethylbut-1-ynyl)-N-(2-sulfamoylphenylsulfonyl)-3-(2-(2,2,2-trifluoroethoxy)-ethoxy)benzamide;
4-(Benzofuran-2-yl)-N-(2-sulfamoylphenylsulfonyl)-3-(2-(2,2,2-trifluoroethoxy)ethoxy)-benzamide;
4-(Phenylethynyl)-N-(2-sulfamoylphenylsulfonyl)-3-(2-(2,2,2-trifluoroethoxy)ethoxy)-benzamide;
4-(3-Methylbut-1-ynyl)-N-(2-sulfamoylphenylsulfonyl)-3-(4,4,4-trifluorobutoxy)-benzamide;
4-(Phenylethynyl)-N-(2-sulfamoylphenylsulfonyl)-3-(3,3,3-trifluoropropoxy)benzamide;
4-(Cyclopentylethynyl)-N-(2-sulfamoylphenylsulfonyl)-3-(3,3,3-trifluoropropoxy)-benzamide;
4-(Cyclopentylethynyl)-N-(2-sulfamoylphenylsulfonyl)-3-(2-(2,2,2-trifluoroethoxy)-ethoxy)benzamide;
4-(Cyclopentylethynyl)-3-(3,3-difluoropropoxy)-N-(2-sulfamoylphenylsulfonyl)-benzamide;
4-(Cyclopentylethynyl)-N-(2-sulfamoylphenylsulfonyl)-3-(4,4,4-trifluorobutoxy)-benzamide;
3-(3,3-Difluoropropoxy)-N-(2-sulfamoylphenylsulfonyl)-4-(p-tolylethynyl)benzamide;
3-(3,3-Difluoropropoxy)-4-(3-methylbut-1-ynyl)-N-(2-sulfamoylphenylsulfonyl)-benzamide;
3-(3-Fluoropropoxy)-4-(3-methylbut-1-ynyl)-N-(2-sulfamoylphenylsulfonyl)benzamide;
3-Methoxy-4-(phenylethynyl)-N-(2-sulfamoylphenylsulfonyl)benzamide;
3-Methoxy-4-(3-methylbut-1-ynyl)-N-(2-sulfamoylphenylsulfonyl)benzamide;
4-Hydroxy-benzene-1,2-disulfonic acid 2-amide 1-(4-benzofuran-2-yl-benzoylamide);
4-Methoxy-benzene-1,2-disulfonic acid 2-amide 1-(4-benzofuran-2-yl-benzoylamide);
4-Hydroxy-benzene-1,2-disulfonic acid 2-amide 1-[4-(3,3-dimethyl-but-1-ynyl)-benzoylamide];
4-Methoxy-benzene-1,2-disulfonic acid 2-amide 1-[4-(3,3-dimethyl-but-1-ynyl)-benzoylamide] and
3-Methoxy-N-(2-sulfamoylphenylsulfonyl)-4-(p-tolylethynyl)benzamide or a pharmaceutically acceptable salts thereof.

As used herein, “alkyl”, used alone or as a suffix or prefix, denotes both branched and straight chain saturated aliphatic hydrocarbon groups having the specified number of carbon atoms. For example, “C₀₋₆ alkyl” denotes either a direct bond (C₀) or an alkyl group having 1, 2, 3, 4, 5 or 6 carbon atoms. Thus a group such as C₀₋₆ alkylCN may represent simply a CN group (C₀) or a C₁₋₆ alkylCN group such as —CH₂CN or —CH₂CH₂CN.

Thus a group such as C₀₋₆alkylheteroaryl may represent simply a heteroaryl group (C₀) or a C₁₋₆alkylheteroaryl group such as —CH₂-heteroaryl or —CH₂CH₂-heteroaryl.

In this way combinations may be formed of any of the herein defined groups, e.g. C₀₋₆alkyl that is covalent bonded to another herein defined group e.g. aryl is forming C₀₋₆alkylaryl.

Examples of alkyl include, but are not limited to, methyl, ethyl, n-propyl, i-propyl, n-butyl, i-butyl, sec-butyl, t-butyl, pentyl, and hexyl. For the avoidance of doubt, where two or more alkyl moieties are present in a substituent, the alkyl moieties may be the same or different.

As used herein, “alkenyl” used alone or as a suffix or prefix denotes an alkyl group as defined above that contains one or more carbon-carbon double bonds. For example, “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used alone or as a suffix or prefix denotes an alkyl group as defined above that contains one or more carbon-carbon triple bonds. For example, “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, the term “aryl” refers to an aromatic ring structure made up of from 5 to 14 carbon atoms. Ring structures containing 5, 6, 7 and 8 carbon atoms would be single-ring (monocyclic) aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes—unless stated to the contrary—polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 4 to 12 carbons atoms.

As used herein, “cycloalkynyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, azetidinyl, pyrazolidinyl, piperidyl, piperidin-2,6-dionyl, piperidin-2-onyl, perhydroazepinyl (hexamethylene iminyl), piperazinyl, morpholinyl, thiomorpholinyl, S-oxothiomorpholinyl, S,S-dioxothiomorpholinyl, 1,3-dioxolanyl, 1,4-dioxanyl, pyrrolidinyl, imidazolidinyl, imidazol-2-onyl, pyrrolidin-2-onyl, tetrahydrofuranyl, tetrahydrothienyl, S,S-dioxotetrahydrothienyl (tetramethylenesulfonyl), dithiolanyl, thiazolidinyl, oxazolidinyl, tetrahydropyranyl and pyrazolinyl moieties. In one embodiment, a 5- to 8-membered heterocyclyl moiety is morpholinyl, tetrahydrofuranyl or S,S-dioxotetrahydrothienyl.

As used herein, “heteroaryl” or “heteroaromatic” refers to an aromatic heterocycle having at least one heteroatom ring member such as sulfur, oxygen, or nitrogen provided that no single ring contains more than three nitrogen atoms. Heteroaryl groups include—unless otherwise stated—monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, indazolyl, 1,2,4-thiadiazolyl, isothiazolyl, thiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms provided that no single ring contains more than three nitrogen atoms. In some embodiments, the heteroaryl or heteroaromatic group has 1 heteroatom. In one embodiment, a 5- or 6-membered heteroaryl moiety is pyrrolyl, thienyl, furanyl, pyridyl, pyrimidinyl, oxazolyl, thiazolyl or pyrazolyl moiety.

For the avoidance of doubt, although the above definitions of heteroaryl and heterocyclyl groups refer to an “N” moiety which can be present in the ring, as will be evident to a skilled chemist the N atom will carry a hydrogen atom (or will carry a substituent as defined above) if it is attached to each of the adjacent ring atoms via a single bond.

As used herein, “L¹ and L²” independently refer to a bond or a 1-7 membered non-aromatic linking group containing 0-2 heteroatoms selected from O, N, and S, said linking group optionally containing CO, S(O)ₙ, C═C or an acetylenic group, and optionally being substituted with one or more R⁸. Examples include but are not limited to —O—, —NH—, —S—, —S(O)—, —S(O)₂—, —CH₂—, —C(O)—, —CH₂CH₂—, —CH═CH—, —C≡C—, —OCH₂—, —CH₂O—, —SCH₂, CH₂S—, —S(O)CH₂—, —CH₂S(O)—, —S(O)₂CH₂—, —CH₂S(O)₂—, —NHCH₂—, —CH₂NH—, —C(O)CH₂—, —CH₂C(O)—, —C(O)O—, —OCH₂CH₂—, —CH₂OCH₂, —CH₂CH₂O—, —CH═CHCH₂—, CH₂CH═CH—, —CH₂S(O)₂CH₂—, —CH₂C≡C—, —C≡CCH₂—, —NHCHMeCH₂—, —(CH₂)₃—, —(CH₂)₄—, —(CH₂)₅—, —CH₂CH═CHCH₂—, —O(CH₂)₃O— and —CH₂NHC(O)—.

As used herein, a C₁₋₆alkoxy moiety is a said C₁₋₆alkyl moiety attached to an oxygen atom. Examples include methoxy and ethoxy.

Examples of bicyclic ring systems in which the two rings are fused together include naphthyl, indanyl, quinolyl, tetrahydroquinolyl, benzofuranyl, indolyl, isoindolyl, indolinyl, benzofuranyl, benzothienyl, indazolyl, benzimidazolyl, benzthiazolyl, benzmorpholinyl, isoquinolyl, chromanyl, indenyl, quinazolyl, quinoxalyl, isocromanyl, tetrahydronaphthyl, pyrido-oxazolyl, pyridothiazolyl, dihydrobenzofuranyl, 1,3-benzodioxolyl, 2,3-dihydro-1,4-benzodioxinyl, 1,3-benzodioxinyl and 3,4-dihydro-isochromenyl. In one embodiment, a bicyclic fused ring system is a naphthyl, indanyl, indolyl, benzofuranyl, benzothienyl, benzthiazolyl, benzmorpholinyl, pyrido-oxazolyl, pyridothiazolyl or dihydrobenzofuranyl moiety.

Examples of tricyclic ring systems in which the three rings are fused together include xanthenyl, carbazolyl, acridinyl, phenothiazinyl, phenoxazinyl, dibenzofuranyl, dibenzothienyl, S,S,-dioxodibenzothienyl, fluorenyl, phenanthrenyl and anthracenyl. In one embodiment, a tricyclic fused ring system is a dibenzofuranyl or S,S,-dioxodibenzothienyl moiety.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as selective inhibitors of the microsomal prostaglandin E synthase-1 enzyme, and may therefore be beneficial in the treatment or prophylaxis of pain and of inflammatory diseases and conditions. Furthermore, by selectively inhibiting the pro-inflammatory PGE2, it is believed that compounds of the invention would have a reduced potential for side effects associated with the inhibition of other prostaglandins by conventional non-steroidal anti-inflammatory drugs, such as gastrointestinal and renal toxicity.

More particularly, the compounds of formula (I) and their pharmaceutically acceptable salts may be used in the treatment of osteoarthritis, rheumatoid arthritis, acute or chronic pain, neuropathic pain, apnea, sudden infant death (SID), wound healing, cancer, benign or malignant neoplasias, stroke, atherosclerosis and Alzheimer's disease.

Even more particularly, the compounds of formula (I) and their pharmaceutically acceptable salts may be used in the treatment of osteoarthritis, rheumatoid arthritis, benign or malignant neoplasias or acute or chronic pain.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

In a further aspect, the present invention provides the use of a compound of formula (I) or a pharmaceutically acceptable salt thereof as hereinbefore defined in the manufacture of a medicament for the treatment of human diseases or conditions in which modulation of microsomal prostaglandin E synthase-1 activity is beneficial.

In a further aspect, the present invention provides the use of a compound of formula (I) or a pharmaceutically acceptable salt thereof as hereinbefore defined in the manufacture of a medicament for use in the treatment of an inflammatory disease or condition.

In a further aspect, the present invention provides the use of a compound of formula (I) or a pharmaceutically acceptable salt thereof as hereinbefore defined in the manufacture of a medicament for use in treating osteoarthritis, rheumatoid arthritis, acute or chronic pain, neuropathic pain, apnea, SID, wound healing, cancer, benign or malignant neoplasias, stroke, atherosclerosis or Alzheimer's disease.

In a further aspect, the present invention provides the use of a compound of formula (I) or a pharmaceutically acceptable salt thereof as hereinbefore defined in the manufacture of a medicament for use in treating acute or chronic pain, nociceptive pain, neuropathic pain, apnea, sudden infant death (SID), atherosclerosis, cancer, aneurysm, hyperthermia, myositis, Alzheimer's disease or arthritis.

In one embodiment of the invention, where cancer is mentioned in any aspect or embodiment of this specification, the cancer is colorectal cancer or lung cancer.

In one embodiment of the invention, where cancer is mentioned in any aspect or embodiment of this specification, the cancer is lung cancer.

In a further aspect, the present invention provides the use of a compound of formula (I) or a pharmaceutically acceptable salt thereof as hereinbefore defined in the manufacture of a medicament for use in treating osteoarthritis, rheumatoid arthritis, benign or malignant neoplasias or acute or chronic pain.

In another aspect, the invention provides a compound of formula (I) or a pharmaceutically acceptable salt thereof as hereinbefore defined for use as a medicament.

In another aspect, the invention provides a compound of formula (I) or a pharmaceutically acceptable salt thereof as hereinbefore defined for the treatment of diseases or conditions in which modulation of microsomal prostaglandin E synthase-1 activity is beneficial.

In another aspect, the invention provides a compound of formula (I) or a pharmaceutically acceptable salt thereof as hereinbefore defined for the treatment of an inflammatory disease or condition.

In another aspect, the invention provides a compound of formula (I) or a pharmaceutically acceptable salt thereof as hereinbefore defined for the treatment of osteoarthritis, is rheumatoid arthritis, acute or chronic pain, neuropathic pain, apnea, SID, wound healing, cancer, benign or malignant neoplasias, stroke, atherosclerosis or Alzheimer's disease.

In another aspect, the invention provides a compound of formula (I) or a pharmaceutically acceptable salt thereof as hereinbefore defined for the treatment of osteoarthritis, rheumatoid arthritis, benign or malignant neoplasias or acute or chronic pain.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, a disease or condition in which modulation of microsomal prostaglandin E synthase-1 activity is beneficial which comprises administering to a patient in need thereof a therapeutically effective amount of a compound of formula (I) or a pharmaceutically acceptable salt thereof as hereinbefore defined.

The invention still further provides a method of treating, or reducing the risk of, an inflammatory disease or condition which comprises administering to a patient in need thereof a therapeutically effective amount of a compound of formula (I) or a pharmaceutically acceptable salt thereof as hereinbefore defined.

The invention still further provides a method of treating, or reducing the risk of, osteoarthritis, rheumatoid arthritis, acute or chronic pain, neuropathic pain, apnea, SID, wound healing, cancer, benign or malignant neoplasias, stroke, atherosclerosis or Alzheimer's disease which comprises administering to a patient in need thereof a therapeutically effective amount of a compound of formula (I) or a pharmaceutically acceptable salt thereof as hereinbefore defined.

The invention still further provides a method of treating, or reducing the risk of, osteoarthritis, rheumatoid arthritis, benign or malignant neoplasias or acute or chronic pain is which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. The daily dosage of the compound of the invention may be in the range from 0.05 mg/kg to 100 mg/kg.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin) in the form, e.g., of creams, solutions or suspensions; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

Thus, the invention further relates to combination therapies wherein a compound of formula (I) or a pharmaceutically acceptable salt thereof, or a pharmaceutical composition or formulation comprising a compound of formula (I) is administered concurrently, simultaneously, sequentially or separately with another pharmaceutically active compound or compounds selected from the following:

(i) neuropathic pain therapies including lidocain, capsaicin, and anticonvulsants such as gabapentin, pregabalin, and antidepressants such as duloxetine, venlafaxine, amitriptyline, klomipramine, and equivalents and pharmaceutically active isomer(s) and metabolite(s) thereof.
(ii) nociceptive pain therapies including paracetamol, NSAIDS and coxibs, such as celecoxib, etoricoxib, lumiracoxib, valdecoxib, parecoxib, diclofenac, loxoprofen, naproxen, ketoprofen, ibuprofen, nabumeton, meloxicam, piroxicam and opioids such as morphine, oxycodone, buprenorfin, tramadol and equivalents and pharmaceutically active isomer(s) and metabolite(s) thereof.
(iii) migraine therapies including for example almotriptan, amantadine, bromocriptine, butalbital, cabergoline, dichloralphenazone, dihydroergotamine, eletriptan, frovatriptan, lisuride, naratriptan, pergolide, pizotiphen, pramipexole, rizatriptan, ropinirole, sumatriptan, zolmitriptan, zomitriptan, and equivalents and pharmaceutically active isomer(s) and metabolite(s) thereof.
(iv) Alzheimer's therapies including for example donepezil, rivastigmine, galantamine, memantine, and equivalents and pharmaceutically active isomer(s) and metabolite(s) thereof.
(v) stroke therapies including for example thrombolytic therapy with eg activase and desmoteplase, abciximab, citicoline, clopidogrel, eptifibatide, minocycline, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in their respective publication reference(s).

The anti-cancer treatment defined hereinbefore may be applied as a sole therapy or may involve, in addition to the compounds of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, approaches to decrease the function of immune suppressive cells such as regulatory T cells, myeloid-derived suppressor cells or IDO (indoleamine 2,3,-deoxygenase)-expressing dendritic cells, and approaches using cancer vaccines consisting of proteins or peptides derived from tumour-associated antigens such as NY-ESO-1, MAGE-3, WT1 or Her2/neu.

According to this aspect of the invention there is provided a pharmaceutical product comprising a compound of the formula (I) as defined hereinbefore and an additional anti-tumour substance as defined hereinbefore for the conjoint treatment of cancer.

According to this aspect of the invention there is provided a combination suitable for use in the treatment of cancer comprising a compound of formula (I) as defined hereinbefore, or a pharmaceutically acceptable salt thereof, and any one of the anti tumour agents listed under (i)-(ix) above.

Therefore in a further aspect of the invention there is provided a compound of Formula (I), or a pharmaceutically acceptable salt thereof, in combination with an anti-tumour agent selected from one listed under (i)-(ix) herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of formula (I), or a pharmaceutically acceptable salt thereof, in combination with an anti-tumour agent selected from one listed under (i)-(ix) herein above, in association with a pharmaceutically acceptable diluent or carrier.

According to a further aspect of the invention there is provided a pharmaceutical composition which comprises a compound of formula (I), or a pharmaceutically acceptable salt thereof, in combination with an anti-tumour agent selected from one listed under (i)-(ix) herein above, in association with a pharmaceutically acceptable diluent or carrier for use in the treatment of cancer.

According to another feature of the invention there is provided the use of a compound of the formula (I), or a pharmaceutically acceptable salt thereof, in combination with an anti-tumour agent selected from one listed under (i)-(ix) herein above, in the manufacture of a medicament for use in cancer in a warm-blooded animal, such as man.

According to another feature of the invention there is provided a compound of the formula (I), or a pharmaceutically acceptable salt thereof, in combination with an anti-tumour agent selected from one listed under (i)-(ix) herein above for use in the treatment of cancer in a warm-blooded animal, such as man.

Therefore in an additional feature of the invention, there is provided a method of treating cancer in a warm-blooded animal, such as man, in need of such treatment which comprises administering to said animal an effective amount of a compound of formula (I), or a pharmaceutically acceptable salt thereof, in combination with an anti-tumour agent selected from one listed under (i)-(ix) herein above.

According to a further aspect of the present invention there is provided a kit comprising a compound of formula (I), or a pharmaceutically acceptable salt thereof, in combination with an anti-tumour agent selected from one listed under (i)-(ix) herein above.

According to a further aspect of the present invention there is provided a kit comprising:

a) a compound of formula (I), or a pharmaceutically acceptable salt thereof, in a first unit dosage form;
b) an anti-tumour agent selected from one listed under (i)-(ix) herein above; in a second unit dosage form; and
c) container means for containing said first and second dosage forms.

### Methods of Preparation

The present invention further provides a process for the preparation of a compound of formula (I) or a pharmaceutically acceptable salt thereof as defined above which comprises;

### Preparation of Intermediates

The process, wherein A, R¹ and m, unless otherwise specified, are as defined hereinbefore, comprises,

(a) converting a compound of formula II to a compound of formula III, wherein B is an optionally substituted aryl or heteroaryl and alkyl is as defined hereinbefore.

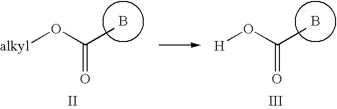

The reaction may be performed by treating an appropriate ester such as a compound of formula II with a suitable base such as lithium hydroxide monohydrate or sodium hydroxide. The reaction may be performed in a suitable solvent or a mixture of solvent such as tetrahydrofuran, water or methanol at a temperature between 0° C. and reflux of the solvent.

(b) converting a compound of formula IV to a compound of formula VI, wherein C is an optionally substituted aryl and R¹⁵ is an optionally substituted alkyl or aryl.

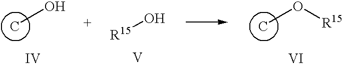

The reaction may be performed by treating a suitable phenol such as a compound of formula IV with a suitable alcohol such as a compound of formula V in the presence of triphenylphosphine and diisopropyl azodicarboxylate or diethyl azodicarboxylate. The reaction may be performed in a suitable solvent such as tetrahydrofuran, dichloromethane, diethyl ether or toluene at a temperature between 0° C. and reflux of the solvent.

(c) deprotection of a compound of formula VII to give a compound of formula VIII, wherein A, R¹ and m are as defined above.

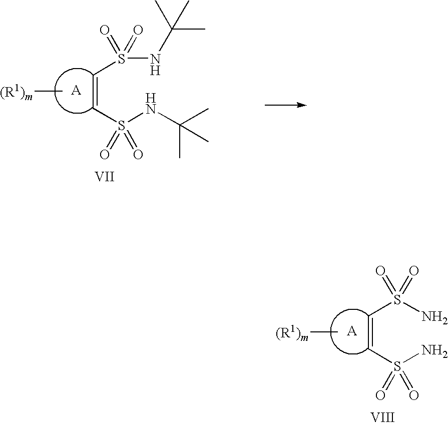

The reaction may be performed by treating a suitable sulfonamide such as a compound of formula VII with boron tribromide in a suitable solvent such as dichloromethane. The reaction may be carried out at a temperature between 0° C. and room temperature.

(d) reaction of a compound of formula IX and a compound of formula X to give a compound of formula XI, wherein C is an optionally substituted aryl and R¹⁶ and R¹⁷ are an optionally substituted alkyl or hydrogen.

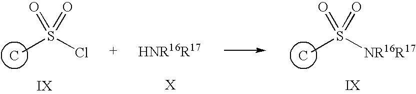

The reaction may be performed by treating a sulfonyl chloride such as a compound of formula IX with a suitable amine such as a compound of formula X. The reaction may be performed in the presences of a tertiary amine such as triethylamine in a suitable solvent such as dichloromethane or tetrahydrofuran at a temperature between 0° C. and room temperature.

(e) a reaction of a compound of formula XII with a compound of formula XIII to a compound of formula XIV, wherein R¹⁸ and R¹⁹ are an optionally substituted aryl or alkyl.

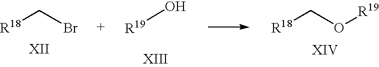

The reaction may be performed by treating an appropriate alcohol such as a compound of formula XIII and an appropriate bromide such as a compound of formula XII in the presence of a suitable base such as potassium carbonate or sodium hydride. The reaction may be performed in a suitable solvent such as acetone, acetonitrile, tetrahydrofuran or N,N-dimethylformamide at a temperature between 0° C. and reflux of the solvent.

(f) a reaction of a compound of formula XV and a compound of formula XVI to give a compound of formula XVII, wherein B is an optionally substituted aryl or heteroaryl and C is an optionally substituted aryl.

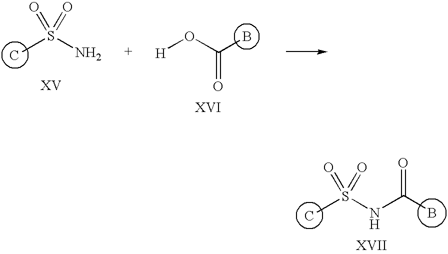

The reaction may be carried out by reacting an appropriate sulfonamide such as a compound of formula XV with an appropriate carboxylic acid such as a compound of formula XVI. If necessary or desired, a base and/or a coupling reagent such as 4-(dimethylamino)pyridine, 1-ethyl-3-(3-dimethylaminopropyl)carbodiimide hydrochloride, 1,1′-carbonyldiimidazole, O-(7-azabenzotriazol-1-yl)-N,N,N′,N′-tetramethyluronium hexafluorophosphate, O-(1H-benzotriazol-1-yl)-N,N,N′,N′-tetramethyluronium, 1-hydroxy-7-azabenzotriazole, 1-hydroxybenzotriazole hydrate, triethylamine or N,N-diisopropylethylamine, and any combinations of the above, may be added. The reaction may be performed in an appropriate solvent such as acetonitrile, dichloromethane, N,N-dimethylformamide or N-methylpyrrolidinone at a temperature between 0° C. and reflux of the solvent.

(g) a reaction of a compound of formula XVIII to a compound of formula XIX, wherein C is an optionally substituted aryl and R²⁰ and R²¹ are an optionally substituted alkyl that together may be linked to form a cyclic amine

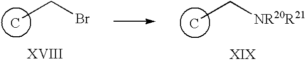

The reaction may be performed by treating an appropriate amine such as 3,3,4,4-tetrafluoropyrrolidine hydrochloride with an appropriate bromide such as a compound of formula XVIII in the presence of a suitable base such as potassium carbonate, sodium hydride or sodium carbonate. The reaction may be performed in a suitable solvent such as acetone, acetonitrile, tetrahydrofuran or N,N-dimethylformamide at a temperature between 0° C. and reflux of the solvent.

(h) a reaction between a compound of formula XX and a compound of formula XXI to give a compound of formula XXII, wherein C is an optionally substituted aryl, R²² is an optionally substituted alkyl and R²³ is methyl or 4-toluene.

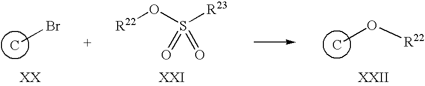

The reaction may be performed by treating an appropriate aryl bromide such as a compound of formula XX with a mesylated or tosylated alcohol such as a compound of formula XXI in the presence of a suitable base such as cesium carbonate, potassium carbonate, sodium hydride, sodium hydroxide or potassium tert-butoxide. The reaction may be performed in a suitable solvent such as N,N-dimethylformamide, toluene, acetonitrile, tetrahydrofuran or dichloromethane at temperature between 0° C. and reflux of the solvent.

(i) a Grignard reaction between a compound of formula XXIII and a compound of formula XXIV to give a compound of formula XXV, wherein R²⁴ and R²⁵ are an optionally substituted alkyl and X is bromine or chlorine.

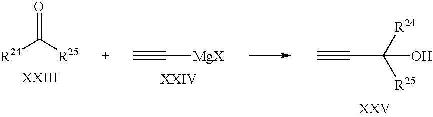

The reaction may be performed by treating a suitable carbonyl compound such as a compound of formula XXIII with a suitable Grignard reagent such as a compound of formula XXIV. The reaction may be performed in a suitable solvent such as tetrahydrofuran or diethyl ether at a temperature between −78° C. and reflux of the solvent.

(j) a reaction of a compound of formula XVIII to give a compound of formula XXVI, wherein C is an optionally substituted aryl.

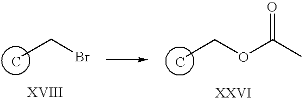

The reaction may be performed by treating an appropriate bromide such as a compound of formula XVIII with a suitable base such as potassium acetate in acetic acid. The reaction may be performed at a temperature between 0° C. and reflux of the solvent.

(k) bromination of a compound of formula XXVII to give a compound of formula XVIII, wherein C is an optionally substituted aryl.

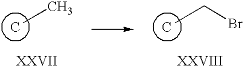

The reaction may be performed by treating a suitable benzyl such as a compound of formula XXVII with a suitable brominating agent such as N-bromosuccinimide and a radical initiator such as 2,2′ azobisisobutyronitrile or benzoyl peroxide. The reaction may be performed in a suitable solvent such as carbon tetrachloride, cyclohexane, benzene or dichloromethane at a temperature between room temperature and reflux of the solvent.

(l) reduction of a compound of formula XXVIII to a compound of formula XXIX, wherein C is an optionally substituted aryl and R²⁶ and R²⁷ are an optionally substituted alkyl.

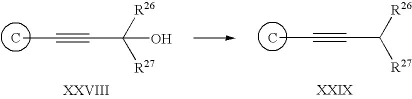

The reaction may be performed by treating a suitable alcohol such as a compound of formula XXVIII with trifluoroacetic acid and triethylsilane. The reaction may be performed in a suitable solvent such as dichloromethane at temperature between 0° C. and room temperature.

(m) oxidation of a compound of formula XXX to a compound of formula XXXI wherein B is an optionally substituted heteroaryl or aryl.

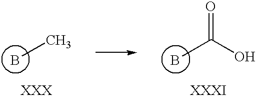

The reaction may be performed by treating a compound such as a compound of formula XXX with a suitable oxidizing agent such as potassium permanganate. The reaction may be performed in a suitable solvent or mixture of solvents such as water and pyridine at a temperature between room temperature and reflux of the solvent.

(n) an oxidation of a compound of formula XX to a compound of formula XXXII, wherein C is an optionally substituted aryl.

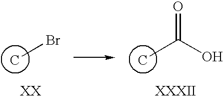

The reaction may be performed by treating a suitable arylbromide such as a compound of formula XX with a suitable base such as n-butyllithium, s-buthyllithium, isopropyl magnesium chloride or treating the arylbromide with magnesium followed by addition of dry ice. The reaction may be performed in a suitable solvent such as tetrahydrofuran, diethyl ether, hexane or toluene at a temperature between −78° C. and room temperature.

(o) a reaction of a compound of formula XXXIII to a compound of formula XXXIV, wherein R²⁸ is an optionally substituted alkyl.

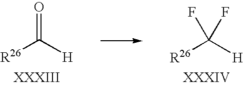

The reaction may be performed by treating a suitable aldehyde such as a compound of formula XXXIII with a suitable fluorinating agent such as bis(2-methyoxyethyl)amino-sulfur trifluoride. The reaction may be performed in a suitable solvent such as dichloromethane, toluene or ethanol at temperature between 0° C. and room temperature.

(p) oxidation of a compound of formula XXXV to a compound of formula XXXIII, wherein R²⁸ is an optionally substituted alkyl.

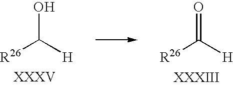

The reaction may be performed by treating a suitable alcohol such as a compound of formula XXXV with a suitable oxidizing agent such as Dess-Martin periodinane (1,1,1-tri(acetoxy)-1,1-dihydro-1,2-benziodoxol-3-(1H)-one). If necessary or desired tert-butanol might be added to the reaction. The reaction may be performed in a suitable solvent such as dichloromethane at a temperature between −20° C. and room temperature.

(q) deprotection of a compound of formula XXXVI to give a compound of formula XXXV, wherein R²⁸ is a optionally protected alkyl.

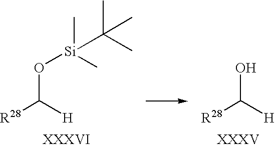

The reaction may be performed by treating a suitable silyl protected alcohol such as a compound of formula XXXVI with tetra-n-butylammonium fluoride. The reaction may be performed in a suitable solvent such as tetrahydrofuran at a temperature between 0° C. and room temperature.

(r) oxidation of a compound of formula XXXVII to a compound of formula IX, wherein C is an optionally substituted aryl.

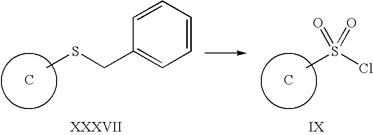

The reaction may be performed by treating a suitable bezylthiol such as a compound of formula XXXVII in formic or acetic acid with chlorine gas. The reaction may be performed in a suitable solvent or mixture of solvents such as dichloromethane and water at a temperature between 0° C. and room temperature.

(s) bezylation of a compound of formula XXXVIII to a compound of formula XXXVII, wherein C is an optionally substituted aryl and halo is bromine or iodine.

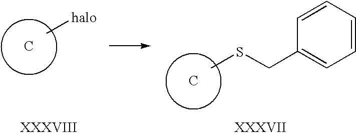

The reaction may be performed by treating a suitable arylhalide such as a compound of formula XXXVIII with benzyl mercaptan. The reaction may be carried out using a suitable palladium catalyst such as tris(dibenzylideneacetone)dipalladium in the presence of a suitable ligand such as 4,5-bis-(diphenylphosphino)-9,9-dimethylxantene or bis(diphenylphosphino)ferrocene. A suitable base such as N,N-diisopropylamine or triethylamine may be used in the reaction, which can be performed at a the temperature between room temperature and reflux of the solvent in a suitable solvent such as dioxane or N,N-dimethylformamide.

(t) a bromination of a compound of formula XXXIX to give a compound of formula XX, wherein C is an optionally substituted aryl.

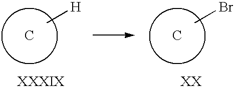

The reaction may be performed by treating a suitable aryl such as a compound of formula XXXIX with a suitable brominating agent such as bromine in acetic acid or with N-bromosuccinimide. The reaction may be performed in a suitable solvent such as acetic acid, N,N-dimethylformamide or acetonitrile at a temperature between room temperature and reflux of the solvent.

Another object of the invention are processes a, b, c or d for the preparation of compounds of general formula I, wherein A, R¹, R², R³ and m, unless otherwise specified, are defined as hereinbefore, and salts thereof. When it is desired to obtain the acid salt, the free base may be treated with an acid such as a hydrogen halide such as hydrogen chloride, sulphuric acid, a sulphonic acid such as methane sulphonic acid or a carboxylic acid such as acetic or citric acid in a suitable solvent such as tetrahydrofuran, diethyl ether, methanol, ethanol, chloroform or dichloromethane or mixtures thereof, the reaction may occur between −30° C. to 50° C.

These processes comprise;

(a) reaction of a compound of formula XL and a compound of formula XLI to a compound of formula I, wherein A, R¹, R², R³, m, L¹, G¹, L² and G² are as defined hereinbefore.

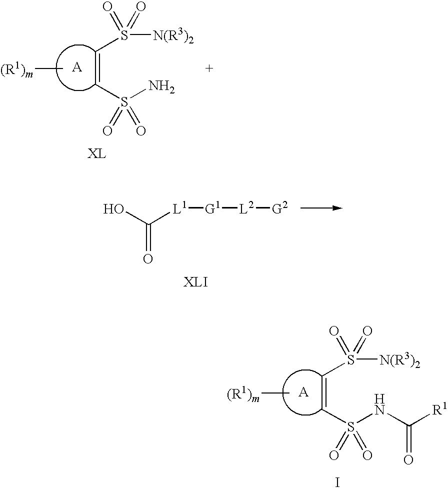

In process (a), the reaction may be carried out by reacting an appropriate sulfonamide such as a compound of formula XL with an appropriate carboxylic acid such as a compound of formula XLI. If necessary or desired, a base and/or a coupling reagent such as 4-(dimethylamino)pyridine (DMAP), 1-ethyl-3-(3-dimethylaminopropyl)carbodiimide hydrochloride (EDC), 1,1′-carbonyldiimidazole (CDI), O-(7-azabenzotriazol-1-yl)-N,N,N′,N′-tetramethyluronium hexafluorophosphate (HATU), O-(1H-benzotriazol-1-yl)-N,N,N′,N′-tetramethyluronium (HBTU), 1-hydroxy-7-azabenzotriazole (HOAT), 1-hydroxybenzotriazole hydrate (HOBT), triethylamine or N,N-diisopropylethylamine (DIEA), and any combinations of the above, may be added. The reaction may be performed in an appropriate solvent such as acetonitrile, dichloromethane, N,N-dimethylformamide or N-methylpyrrolidinone at a temperature between 0° C. to the boiling point of the solvent.

(b) reaction of a compound of formula XLII and a compound of formula XLI to a compound of formula I, wherein A, R¹, R², R³, L¹ and m are as defined hereinbefore, G¹ and G² are an optionally substituted aryl, halo is a halogen and M represents an organo-tin or organo boronic acid group.

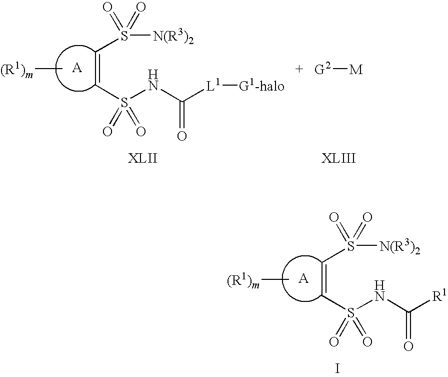

In process (b), the reaction may conveniently be carried out by reaction with an appropriate aryl boronic acid or an aryl boronic ester such as a compound of formula XLIII. The reaction may be carried out using a suitable palladium catalyst such as tetrakis(triphenylphosphine) palladium(0)(Pd(PPh₃)₄), dichloro 1,1′-bis(diphenylphosphino)ferrocene palladium(II)(Pd(dppf)Cl₂), trans-dichlorobis(triphenylphosphine) palladium(II)(Pd(PPh₃)₂Cl₂) or palladium(II) acetate (Pd(OAc)₂) or tris(dibenzylideneacetone)dipalladium(0)(Pd₂(dba)₃) together with a suitable ligand such as P(tert-butyl)₃, 2-(dicyclohexylphosphino)biphenyl, or 2-(2′,6′-dimethoxybiphenyl)-dicyclohexylphosphine, or a nickel catalyst such as nickel on charcoal or Ni(dppe)Cl₂ together with zinc and sodium triphenylphosphinetrimetasulfonate. A suitable base such as an alkyl amine, e.g. triethylamine, or potassium carbonate, sodium carbonate, cesium carbonate, sodium hydroxide or cesium fluoride may be used in the reaction, which can be performed in the temperature range of room temperature and reflux of the solvent, using an oil bath or a microwave oven, in a suitable solvent or solvent mixture such as toluene, tetrahydrofuran, dimethoxyethane/water, N,N-dimethylformamide or dioxane. The boronic acid or boronic ester may be formed in situ, by reaction of the corresponding aryl halide (e.g., the aryl bromide) with an alkyllithium reagent such as butyllithium to form an intermediate aryl lithium species, which then is reacted with a suitable boron compound, e.g., trimethyl borate, tributyl borate or triisopropyl borate.

(c) reaction of a compound of formula XLII to a compound of formula I, wherein A, R¹, R², R³, L¹ and m are as defined hereinbefore, G¹ is an optionally substituted aryl and halo is a halogen.

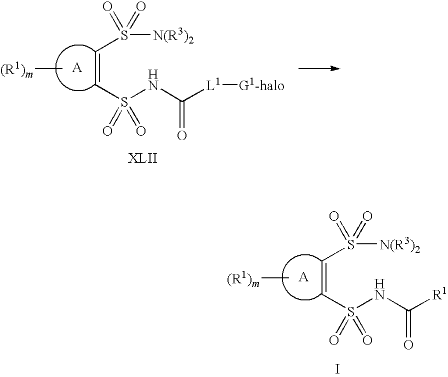

In process (c), the reaction may be carried out by reaction with an appropriate optionally substituted alkyne. The reaction may be carried out using a suitable palladium catalyst such as tetrakis(triphenylphosphine) palladium(0) (Pd(PPh₃)₄), trans-dichlorobis(triphenylphosphine) palladium(II)(PdCl₂(PPh₃)₂) or bis(acetonitrile)dichloropalladium(II) ([PdCl₂(CH₃CN)₂]). The reaction may be performed in the presence of a suitable ligand such as 2-dicyclohexylphosphino-2′,4′,6′-triisopropylbiphenyl (Xphos). The reaction may be performed in the presence of a suitable copper catalyst such as copper(I) iodide. A suitable base such as triethylamine, butylamine, diisopropylamine or cesium carbonate may be used in the reaction, which can be performed in the temperature range of room temperature and reflux of the solvent, using an oil bath or a microwave oven, in a suitable solvent or a mixture of solvents such as N,N-dimethylformamide, dimethyl sulfoxide, acetonitrile, toluene, tetrahydrofuran, dimethoxyethane/water or dioxane.

(d) reaction of a compound of formula XLIV to a compound of formula I, wherein A, R², R³, L¹ and m are as defined hereinbefore and R¹ is hydroxy.

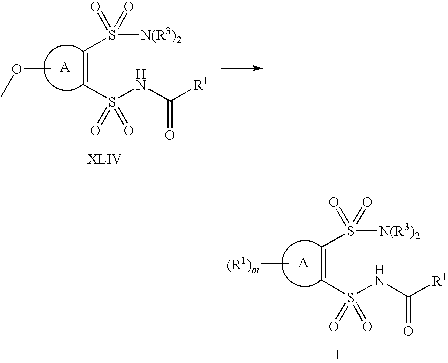

The reaction may be performed by deprotection a suitable methyl ether such as a compound of formula XLIV with boron tribromide in a suitable solvent such as dichloromethane. The reaction may be carried out at a temperature between −78° C. and room temperature.

and optionally after (a), (b), (c) or (d) carrying out one or more of the following:
converting the compound obtained to a further compound of the invention
forming a pharmaceutically acceptable salt of the compound.

Specific processes for the preparation of compounds of Formula (I) are disclosed within the Examples section of the present specification. Such processes form an aspect of the present invention.

The necessary starting materials are either commercially available, are known in the literature or may be prepared using known techniques. Specific processes for the preparation of certain key starting materials are disclosed within the Examples section of the present specification and such processes form an aspect of the present invention.

Certain intermediates are novel. Such novel intermediates form another aspect of the invention.

Compounds of formula (I) can be converted into further compounds of formula (I) using standard procedures.

It will be appreciated by those skilled in the art that in the processes of the present invention certain functional groups such as hydroxyl or amino groups may need to be protected by protecting groups. Thus, the preparation of the compounds of formula (I) may involve, at an appropriate stage, the addition and/or removal of one or more protecting groups.

The protection and deprotection of functional groups is described in ‘Protective Groups in Organic Chemistry’, edited by J. W. F. McOmie, Plenum Press (1973) and ‘Protective is Groups in Organic Synthesis’, 3rd edition, T. W. Greene and P. G. M. Wuts, Wiley-Interscience (1999).

The present invention will now be further explained by reference to the following illustrative examples.

### General Methods

All solvents used were commercially available and were used without further purification. Reactions were typically run using anhydrous solvents under an inert atmosphere of nitrogen or argon.

Starting materials used were either available from commercial sources or prepared according to literature procedures and had experimental data in accordance with those reported.

Chemical shifts are given in ppm down- and upfield from TMS (0.00 ppm). The following reference signals were used: TMS δ 0.00, or the residual solvent signal of DMSO-d₆ δ 2.49, CD₃OD δ 3.30, acetone-d₆ 2.04 or CDCl₃ δ 7.25 (unless otherwise indicated).

Resonance multiplicities are denoted s, d, t, quint, m, br and app for singlet, doublet, triplet, quartet, multiplet, broad and apparent, respectively.

¹H and ¹³C NMR spectra were recorded on a Varian Unity+ 400 NMR spectrometer operating at 400 MHz for ¹H and 100 MHz for ¹³C equipped with a 5 mm BBO probehead with Z-gradients.

Alternatively, on a Bruker av400 NMR spectrometer operating at 400 MHz ¹H and 100 MHz for ¹³C equipped with a 3 mm flow injection SEI ¹H/D-13C probehead with Z-gradients, using a BEST 215 liquid handler for sample injection.

Alternatively, on a Bruker DPX400 NMR spectrometer operating at 400 MHz for ¹H, 376 MHz for ¹⁹F, and 100 MHz for ¹³C equipped with a 4-nucleus probehead with Z-gradients.

Alternatively, on a Bruker DRX600 NMR spectrometer, operating at 600 MHz for ¹H, 150 MHz for ¹³C, and 60 MHz for ¹⁵N equipped with a 5 mm TXI probehead with Z-gradients, or equipped with a 5 mm BBO probehead with Z-gradients, or equipped with a 2.5 mm BBI probehead with Z-gradients.

Alternatively, on a Bruker 500 MHz Avance III NMR spectrometer, operating at 500 MHz for ¹H, 125 MHz for ¹³C, and 50 MHz for ¹⁵N equipped with a 5 mm TXI probehead with Z-gradients.

Alternatively, ¹H and ¹³C NMR spectra were recorded at 400 MHz for proton and 100 MHz for carbon-13 on a Varian Mercury Plus 400 NMR Spectrometer equipped with a Varian 400 ATB PFG probe.

LC-MS analyses were performed on a LC-MS system consisting of a Waters Alliance 2795 HPLC, a Waters PDA 2996 diode array detector, a Sedex 85 ELS detector and a ZQ single quadrupole mass spectrometer. The mass spectrometer was equipped with an electrospray ion source (ES) operated in positive and negative ion mode. The capillary voltage was set to 3.3 kV and the cone voltage to 28 V, respectively. The mass spectrometer scanned between m/z 100-800 with a scan time of 0.3 s. The diode array detector scanned from 200-400 nm. The temperature of the ELS detector was adjusted to 40° C. and the pressure was set to 1.9 bar. Separation was performed on an Gemini C18, 3.0 mm×50 mm, 3 μm, (Phenomenex) run at a flow rate of 1 mL/min. A linear gradient was applied starting at 100% A (A: 10 mM ammonium acetate in 5% acetonitrile) ending at 100% B (B: acetonitrile), followed by 100% B. The column oven temperature was set to 40° C.

alternatively, LC-MS analyses were recorded on a Waters MS consisting of an Alliance 2795 (LC) and Waters Micromass ZQ detector at 120° C. The mass spectrometer was equipped with an electrospray ion source (ES) operated in a positive or negative ion mode. The mass spectrometer was scanned between m/z 100-1000 with a scan time of 0.3 s;

Purity analyses were performed on an Agilent HP1100 system consisting of a G1322A Micro Vacuum Degasser, a G1312A Binary Pump, a G1367 A Well-Plate Autosampler, a G1316A Thermostatted Column Compartment, a G1315C Diode Array Detector and a 6120, G1978B mass spectrometer. The mass spectrometer was configured with an atmospheric pressure chemical ionisation (APCI) ion source operated in positive and negative ion mode. The APCI corona was set to 5.0 μA. The capillary voltage was set to 2.0 kV. Desolvation temperature was at 350° C. and desolvation gas at 5 L/min. The mass spectrometer was scanning between m/z 100-1000. The column used was a Gemini C18 3.0×50, 3 μm (Phenomenex) run at a flow rate of 1.0 mL/min. The column oven temperature was set to 40° C. The diode array detector scanned from 200-400 nm. The purity method consisted of two or three parts: firstly a 3 minute column wash was applied (this part is optional), secondly a blank run was performed and finally the sample was analysed. A linear gradient was used for both the blank and the sample, starting at 100% A (A: 10 mM ammonium acetate in 5% acetonitrile) and ending at 95% B (B: acetonitrile) after 3.0 minutes, then 95% B during 1 min stop at 4.0 min. Integration was on at 0 to 4.9 min. The blank run was subtracted from the sample run at the wavelengths 220 nm, 254 nm, 290 nm and from the chromatograms of the mass spectrometer in positive and negative mode.

GC-MS analyses were performed on a GC/DIP-MS system supplied by Agilent Technologies. Consisting of a GC 6890N, G1530N, a G2614A Auto-sampler, G2613A injector and a G2589N mass spectrometer. The mass spectrometer was equipped with a Direct Inlet Probe (DIP) interface manufactured by SIM GmbH. The mass spectrometer was equipped with an electron impact (E1) ion source and the electron voltage was set to 70 eV. The mass spectrometer scanned between m/z 50-550 and the scan speed was set to 2.91 scan/s. Solvent delay was set from 0 min to 0.8 min. The column used was a DB-5 MS, ID 0.18 mm×10m, 0.18 μm (J&W Scientific). When introduced by GC, a linear temperature gradient was applied starting at 40-90° C. (depending on method), a gradient of 40° C./minute, ending at 300° C.

HPLC analyses were performed on an Agilent HP 1100 system consisting of a G1379A Micro Vacuum Degasser, a G1312A Binary Pump, a G1367A Well-Plate Autosampler, a G1316A Thermostated Column Compartment and a G1315B Diode Array Detector. The diode array detector was scanned from 200 to 400 nm, step and peak width were set to 2 nm and 0.01 min, respectively. The column used was a Gemini C18, 3.0×50 mm, 3.0 μm, 110 Å run at a flow rate of 1.0 mL/min. The column oven temperature was set to 40° C. A linear gradient was applied, starting at 100% A (A: 10 mM ammonium acetate in 5% acetonitrile) and ending at 100% B (B: acetonitrile) then 95% B.

Alternatively, using a G1322A Micro Vacuum Degasser, a G1312A Binary Pump, a G1367A Well-Plate Autosampler, a G1316A Thermostated Column Compartment and a G1315B Diode HPLC analyses were performed on an Agilent HP1100 system consisting of a Array Detector. The diode array detector was scanned from 200 to 400 nm, step and peak width were set to 2 nm and 0.01 min, respectively. The column used was a Gemini C18, 3.0×50 mm, 3.0 110 Å run at a flow rate of 1.0 mL/min. The column oven temperature was set to 40° C. A linear gradient was applied, starting at 100% A (A: 10 mM ammonium acetate in 5% acetonitrile) and ending at 100% B (B: acetonitrile) then 95% B.

Alternatively, HPLC analyses were performed on a Water 600 Controller system with a Waters 717 6 min, holding at 90% C for 4 min and then ending back at 95% A. The column was at ambient temperature with the flow rate of 1.0 mL/min. The Diode Array Detector was scanned from 200-400 nm.

Microwave heating was performed in a CEM Discover LabMate or in a Creator™, Initiator™ or Smith Synthesizer™ Single-mode microwave cavity producing continuous irradiation at 2450 MHz at the indicated temperature in the recommended microwave tubes.

Thin layer chromatography (TLC) was performed on Merck TLC-plates (Silica gel 60 F₂₅₄) and UV visualized the spots.

Alternatively, on Alugram® (Silica gel 60 F₂₅₄) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), vanillin (generated by dissolving about 1 g vanillin in 100 mL 10% H₂SO₄), ninhydrin (available commercially from Aldrich), or Magic Stain (generated by thoroughly mixing 25 g (NH₄)₆Mo₇O₂₄.4H₂O, 5 g (NH₄)₂Ce(IV)(NO₃)₆, 450 mL H₂O and 50 mL concentrated H₂SO₄) to visualize the compound.

Column chromatography was preformed using 40-63 μm (230-400 mesh) silica gel from Silicycle following analogous techniques to those disclosed in Still, W. C.; Kahn, M.; and Mitra, M. Journal of Organic Chemistry, 1978, 43, 2923-2925.

Alternatively, column chromatography was performed on a Combi Flash® Companion™ using RediSep™ normal-phase columns or using Merck Silica gel 60 (0.040-0.063 mm)

Typical solvents used for column chromatography were mixtures of chloroform/methanol, dichloromethane/methanol, heptane/ethyl acetate, chloroform/methanol/ammonia (aq.) and dichloromethane/methanol/ammonia (aq.).

Preparative chromatography was performed on either a Waters Prep LC 4000 System using a Waters 2487 Diode Array or on a Waters LC Module 1 plus. The column used was either a Waters XTerra Prep C₁₈, 5 nm, 30×100 mm (flow rate 40 mL/min) or a Phenomenex Luna C₁₈, 5 nm, 21.6×250 mm (flow rate 20 mL/min) Narrow gradients with acetonitrile/water, with the water containing either 0.1% trifluoroacetic acid or 10 mM ammonium acetate, were used to elute the compound in a total run time between 20-30 min.

Alternatively, preparative chromatography was run on a Waters FractionLynx system with a Autosampler combined Automated Fraction Collector (Waters 2767), Gradient Pump (Waters 2425), Make Up Pump (Waters 515), Waters Passive Splitter, Column Switch (Waters SFO), PDA (Waters 2996) and Waters ZQ mass spectrometer. Column; XBridge™ Prep C8 5 μm OBD™ 19×250 mm, with guard column; XTerra® Prep MS C8 10 μm 19×10 mm Cartridge. A gradient from within 100% A (95% 0.1 M ammonium acetate in MilliQ water and 5% acetonitrile) to 100% B (100% acetonitrile) was applied for LC-separation at flow rate 20 mL/min. The PDA was scanned from 210-350 nm. The ZQ mass spectrometer was run with ESI in positive or negative mode. The Capillary Voltage was 3 kV and the Cone Voltage was 30V. Mixed triggering, UV and MS signal, determined the fraction collection.

Naming was done with CambridgeSoft MedChem ELN v2.1 or ChemDraw Ultra 7.0.

### EXAMPLE 1

### 4-(Benzyloxy)-3-(cyclopropylmethoxy)-N-(2-sulfamoylphenylsulfonyl)benzamide

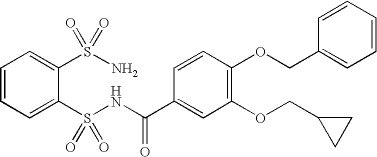

4-(Benzyloxy)-3-(cyclopropylmethoxy)benzoic acid (0.038 g, 0.13 mmol), N-(3-dimethylaminopropyl)-N′-ethylcarbodiimide hydrochloride (0.037 g, 0.19 mmol) and 4-(dimethylamino)pyridine (0.023 g, 0.19 mmol) were added to a solution of benzene-1,2-disulfonamide (0.030 g, 0.13 mmol) in N,N-dimethylformamide (6 mL) at room temperature. After stirring over night the reaction mixture was partitioned between water and ethyl acetate. The aqueous phase was acidified with 2.0 M aqueous hydrochloric acid and extracted with ethyl acetate. The organic phase was dried over magnesium sulfate and the solvent was evaporated. Purification by preparative HPLC gave 0.029 g (44% yield) of the title compound. ¹H NMR (400 MHz, DMSO-d₆) δ ppm 8.31 (d, 1H), 8.13 (d, 1H), 7.80-7.94 (m, 2H), 7.47-7.54 (m, 2H), 7.42-7.47 (m, 2H) 7.36-7.42 (m, 4H), 7.28-7.36 (m, 1H), 7.10 (d, 1H), 5.20 (s, 2H) 3.86 (d, 2H), 1.14-1.31 (m, 1H), 0.50-0.63 (m, 2H), 0.25-0.38 (m, 2H); MS (ESI) m/z 515 [M−1]⁻.

### a) 4-(Benzyloxy)-3-(cyclopropylmethoxy)benzoic acid

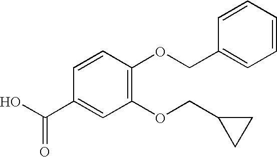

A solution of lithium hydroxide (0.022 g, 0.53 mmol in water (1 mL) was added to a solution of methyl 4-(benzyloxy)-3-(cyclopropylmethoxy)benzoate (0.055 g, 0.18 mmol) in tetrahydrofuran (6 mL) at room temperature. After stirring for 3 days the reaction mixture was partitioned between water and ethyl acetate. The aqueous phase was acidified with 2 M aqueous hydrochloric acid and extracted with ethyl acetate. The organic phase was dried over magnesium sulfate and the solvent was evaporated to give 0.039 g (75% yield) of the title compound. ¹H NMR (400 MHz, DMSO-d₆) δ ppm 7.58 (dd, 1H), 7.49-7.55 (m, 3H), 7.43-7.49 (m, 2H), 7.36-7.43 (m, 1H), 7.18 (d, 1H), 5.26 (s, 2H), 3.93 (d, 2H), 1.20-1.36 (m, 1H), 0.57-0.67 (m, 2H), 0.34-0.45 (m, 2H); MS (ESI) m/z 297 [M−1]⁻.

### b) Methyl 4-(benzyloxy)-3-(cyclopropylmethoxy)benzoate

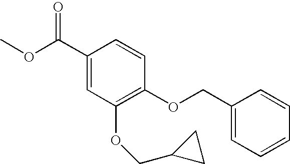

Diisopropyl azodicarboxylate (0.186 mL, 0.94 mmol), triphenylphosphine (0.248 g, 0.94 mmol) and cyclopropanemethanol (0.061 mL, 0.76 mmol) were added to a solution of methyl 4-(benzyloxy)-3-hydroxybenzoate (0.145 g, 0.56 mmol, Chunlin He et al, J. Org. Chem., 2008, 73(15), 5872-5880,) in tetrahydrofuran (8 mL). The reaction mixture was stirred over night and concentrated. Purification by column chromatography, using heptane/ethyl acetate (10:1) as the eluent, gave 0.057 g (32% yield) of the title compound. GC MS (EI) m/z 312 [M]^{+•}.

### c) Benzene-1,2-disulfonamide

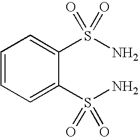

Boron tribromide (7.80 mL, 82.50 mmol) was added to a solution of N1,N2-di-tert-butylbenzene-1,2-disulfonamide (57.5 g, 165.00 mmol) in dichloromethane (300 mL) at room temperature under an atmosphere of nitrogen. The resulting mixture was stirred over night. Dichloromethane (200 mL) and additional boron tribromide (3.9 mL, 41.25 mmol) were added and stirring was continued for 1 hour. The reaction slurry was cooled to 10° C. and quenched with water (150 mL). The pH of the aqueous phase was adjusted to basic with 4 M sodium hydroxide (42 mL). The mixture was filtered, the product washed with water and dichloromethane and dried in a vacuum cabinet at 50° C. over night to give 37 g (95% yield) of the title compound. ¹H NMR (400 MHz, DMSO-d₆): δ ppm 8.14 (dd, 2H), 7.86 (dd, 2H), 7.27 (s, 4H); MS (ESI) m/z 235.1 [M−1]⁻

### d) N1,N2-di-tert-Butylbenzene-1,2-disulfonamide

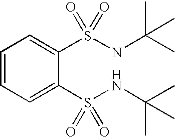

A solution of 1,2-benzenedisulfonyl chloride (56.9 g, 207 mmol) in dichloromethane (1 L) was added to a stirred mixture of tert-butylamine (43.5 mL, 414 mmol) and triethylamine (57.7 mL, 414 mmol) in dichloromethane (1 L) at room temperature. The resulting mixture was stirred at room temperature for 1 hour and hydrochloric acid (3% aqueous solution, 500 mL) was added. The organic phase was separated, washed with brine, dried over anhydrous magnesium sulfate and concentrated under reduced pressure to give 50.0 g (87% yield) of the title compound. ¹H NMR (400 MHz, CDCl₃) δ ppm 8.26 (dd, 2H), 7.70 (dd, 2H), 6.37 (s, 2H), 1.19 (s, 18H).

### EXAMPLE 2

### 3-Methoxy-N-(2-sulfamoylphenylsulfonyl)-4-(p-tolyloxymethyl)benzamide

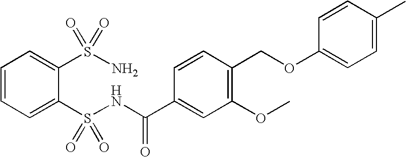

The title compound was synthesized as described for Example 1 in 30% yield, starting from 3-methoxy-4-(p-tolyloxymethyl)benzoic acid. The residue was purified by column chromatography, using ethyl acetate followed by ethyl acetate/methanol (100:1 and 10:1) as the eluent, followed by an purification by preparative HPLC. ¹H NMR (400 MHz, DMSO-d₆) δ ppm 8.29-8.40 (m, 1H), 8.12-8.20 (m, 1H), 7.81-7.97 (m, 2H), 7.56 (d, 1H), 7.47-7.53 (m, 1H), 7.38-7.47 (m, 3H), 7.04-7.12 (m, 2H), 6.81-6.91 (m, 2H), 5.05 (s, 2H), 3.88 (s, 3H), 2.22 (s, 3H); MS (ESI) m/z 489 [M−1]⁻.

### a) 3-Methoxy-4-(p-tolyloxymethyl)benzoic acid

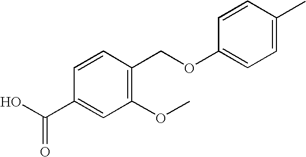

The title compound was synthesized as described for Example 1a) in 18% yield, starting from methyl 3-methoxy-4-(p-tolyloxymethyl)benzoate. MS (ESI) m/z 271 [M−1]⁻.

### b) Methyl 3-methoxy-4-(p-tolyloxymethyl)benzoate

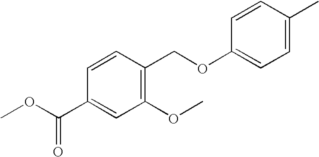

Potassium carbonate (0.693 g, 5.02 mmol) was added to a stirred solution of methyl 4-(bromomethyl)-3-methoxybenzoate (1.0 g, 3.86 mmol) and p-cresol (0.417 g, 3.86 mmol) in anhydrous acetone at room temperature. The reaction mixture was stirred over night, filtered and the filtrate was evaporated. Purification by column chromatography using heptane/ethyl acetate (10:1 and 7:1) as the eluent, gave the crude title compound. GC MS (EI) m/z 286 [M]^{+•}.

### EXAMPLE 3

### 3-(2,2-Difluoroethoxy)-4-(phenylethynyl)-N-(2-sulfamoylphenylsulfonyl)benzamide

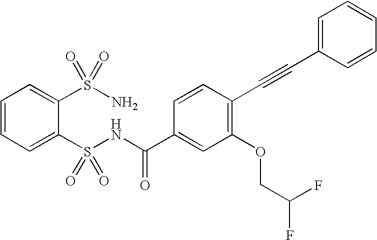

Ethynylbenzene (0.053 g, 0.51 mmol), tetrakis(triphenylphosphine)palladium(0) (0.050 g, 0.04 mmol) and triethylamine (1.792 mL, 12.86 mmol) was added to a solution of 4-bromo-3-(2,2-difluoroethoxy)-N-(2-sulfamoylphenylsulfonyl)benzamide (0.214 g, 0.43 mmol) in N,N-dimethylformamide (8 mL) under an atmosphere of argon. The reaction mixture was stirred at room temperature for 5 min, copper(I) iodide (0.012 g, 0.06 mmol) was added and the reaction mixture was heated at 65° C. After stirring over night the reaction mixture was partitioned between water and ethyl acetate. The aqueous phase was acidified with 2.0 M aqueous hydrochloric acid and extracted with ethyl acetate. The organic phase was dried over magnesium sulfate and the solvent was evaporated. Purification by preparative HPLC gave 67.0 g (30% yield) of the title compound. ¹H NMR (500 MHz, CD₃OD) δ ppm 8.46-8.53 (m, 1H), 8.23-8.31 (m, 1H), 7.80-7.90 (m, 2H,) 7.59 (s, 1H), 7.53-7.57 (m, 2H), 7.49-7.53 (m, 2H), 7.35-7.44 (m, 3H), 6.11-6.43 (m, 1H), 4.39 (td, 2H); MS (ES) m/z 519 [M−1]⁻.

### EXAMPLE 4

### 3-(2,2-Difluoroethoxy)-N-(2-sulfamoylphenylsulfonyl)-4-(p-tolylethynyl)benzamide

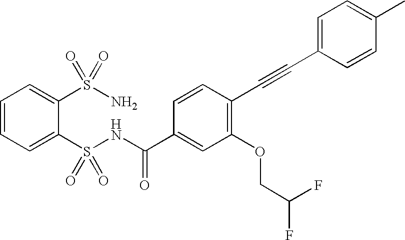

The title compound was synthesized as described for Example 3 in 33% yield, starting from 1-ethynyl-4-methylbenzene. ¹H NMR (400 MHz, DMSO-d₆) δ ppm 8.30-8.41 (m, 1H) 8.09-8.22 (m, 1H), 7.81-7.97 (m, 2H), 7.65 (d, 1H), 7.50-7.62 (m, 2H), 7.45 (s, 2H), 7.37-7.44 (m, 2H), 7.25 (dd, 2H), 6.26-6.69 (m, 1H), 4.47 (td, 2H), 2.27-2.42 (m, 3H); MS (ESI) m/z 533 [M−1]⁻.

### EXAMPLE 5

### 4-(Cyclopentylethynyl)-3-(2,2-difluoroethoxy)-N-(2-sulfamoylphenylsulfonyl)benzamide

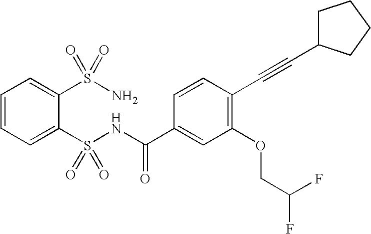

The title compound was synthesized as described for Example 3 in 19% yield, starting from ethynylcyclopentane. ¹H NMR (400 MHz, DMSO-d₆) δ ppm 8.30-8.39 (m, 1H), 8.10-8.19 (m, 1H), 7.82-7.99 (m, 2H), 7.58 (d, 1H), 7.42-7.50 (m, 3H), 7.36-7.42 (m, 1H), 6.24-6.59 (m, 1H), 4.40 (td, 2H), 2.90 (t, 1H), 1.87-1.99 (m, 2H), 1.67-1.78 (m, 2H), 1.51-1.67 (m, 4H); MS (ESI) m/z 511 [M−1]⁻.

### EXAMPLE 6

### 3-(2,2-Difluoroethoxy)-4-(3,3-dimethylbut-1-ynyl)-N-(2-sulfamoylphenylsulfonyl)benzamide

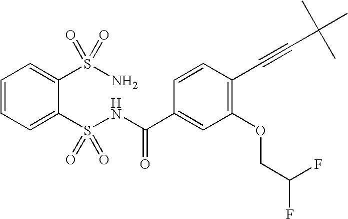

The title compound was synthesized as described for Example 3 in 27% yield, starting from diisopropyl 3,3-dimethylbut-1-ynylboronate. ¹H NMR (500 MHz, DMSO-d₆) δ ppm 8.30-8.39 (m, 1H), 8.13-8.19 (m, 1H), 7.80-7.96 (m, 2H), 7.58 (d, 1H), 7.47 (dd, 1H), 7.42 (s, 2H), 7.38 (d, 1H), 6.25-6.55 (m, 1H), 4.39 (td, 2H), 1.28 (s, 9H); MS (ESI) m/z 499 [M−1]⁻.

### EXAMPLE 7

### 4-(Benzofuran-2-yl)-3-(2,2-difluoroethoxy)-N-(2-sulfamoylphenylsulfonyl)benzamide

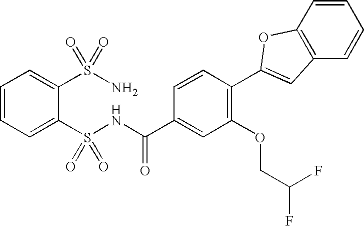

2.0 M Aqueous sodium carbonate (0.625 mL, 1.25 mmol) was added to a mixture of 4-bromo-3-(2,2-difluoroethoxy)-N-(2-sulfamoylphenylsulfonyl)benzamide (0.208 g, 0.42 mmol), benzofuran-2-ylboronic acid (0.135 g, 0.83 mmol), 1,1′-bis(diphenylphosphino)ferrocene-palladium dichloride (0.034 g, 0.04 mmol) in N,N-dimethylformamide (3 mL) under an atmosphere of argon. The reaction mixture was heated in a microwave at 120° C. for 20 min under an atmosphere of argon. The reaction mixture was partitioned between water and ethyl acetate. The aqueous phase was acidified with 2.0 M aqueous hydrochloric acid and extracted with dichloromethane. The organic phase was dried over magnesium sulfate and the solvent was evaporated. Purification by preparative HPLC gave 0.104 g (46% yield) of the title compound. ¹H NMR (500 MHz, DMSO-d₆) δ ppm 8.37 (d, 1H), 8.16 (d, 1H), 8.06 (d, 1H), 7.85-7.95 (m, 2H), 7.77 (s, 1 H), 7.67-7.75 (m, 2H), 7.65 (d, 1H), 7.52 (s, 1H), 7.45 (s, 2H), 7.35-7.41 (m, 1H), 7.29 (t, 1H), 6.53-6.81 (m, 1H), 4.63 (td, 2H); MS (ESI) m/z 535 [M−1]⁻.

### EXAMPLE 8

### N-(2-Sulfamoylphenylsulfonyl)-4-(o-tolylethynyl)benzamide

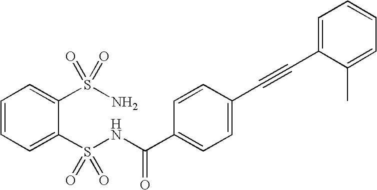

The title compound was synthesized as described for Example 3 in 33% yield, starting from 4-bromo-N-(2-sulfamoylphenylsulfonyl)benzamide and 1-ethynyl-2-methylbenzene. ¹H NMR (500 MHz, DMSO-d₆) δ ppm 8.35 (d, 1H), 8.15 (d, 1H), 7.93 (d, 2H), 7.84-7.91 (m, 2H), 7.65 (d, 2H), 7.53 (d, 1H), 7.42 (br. s., 2H), 7.34 (d, 2H), 7.25 (dd, 1H), 2.47 (s, 3H); MS (ESI) m/z 453 [M−1]⁻.

### a) 4-Bromo-N-(2-sulfamoylphenylsulfonylbenzamide

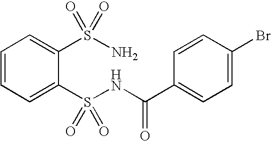

Benzene-1,2-disulfonamide (118 mg, 0.5 mmol), 4-bromobenzoic acid (131 mg, 0.65 mmol), N-(3-dimethylaminopropyl)-N′-ethylcarbodiimide hydrochloride (124 mg, 0.65 mmol) and 4-(dimethylamino)pyridine (183 mg, 1.5 mmol) were mixed in N,N-dimethylformamide (3 mL) and the reaction mixture was stirred for 3 hours. The reaction mixture was diluted with water (0.5 mL) and filtered. The filtrate was purified by HPLC to give 91 mg (43% yield) of the title compound. ¹H NMR (400 MHz, DMSO-d₆) δ ppm 8.14 (d, 1H), 7.98 (d, 1H), 7.80 (d, 2H), 7.54-7.67 (m, 2H), 7.51 (d, 2H), 7.42 (s, 2H); MS (ESI) m/z 419, 421 [M+1]⁺, MS (ESI) m/z 417, 419 [M−1]⁻.

### EXAMPLE 9

### N-(2-sulfamoylphenylsulfonyl)-4-(p-tolylethynyl)benzamide

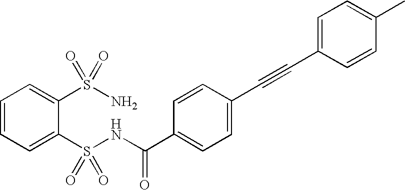

The title compound was synthesized as described for Example 3 in 38% yield, starting from 4-bromo-N-(2-sulfamoylphenylsulfonyl)benzamide and 1-ethynyl-4-methylbenzene. ¹H NMR (500 MHz, DMSO-d₆) δ ppm 8.35 (d, 1H), 8.15 (d, 1H), 7.82-7.98 (m, 4H), 7.62 (d, 2H), 7.47 (d, 2H), 7.42 (br. s., 2H), 7.26 (d, 2H), 2.34 (s, 3H); MS (ES) m/z 453 [M−1]⁻.

### EXAMPLE 10

### 4-((4-Chlorophenyl)ethynyl)-N-(2-sulfamoylphenylsulfonyl)benzamide

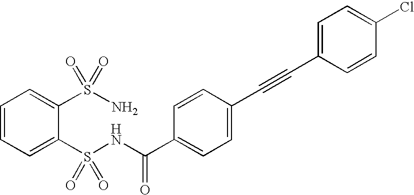

The title compound was synthesized as described for Example 3 in 21% yield, starting from 4-bromo-N-(2-sulfamoylphenylsulfonyl)benzamide and 1-chloro-4-ethynylbenzene. ¹H NMR (500 MHz, DMSO-d₆) δ ppm 8.34 (d, 1H), 8.15 (d, 1H), 7.93 (d, 2H), 7.82-7.91 (m, 2H), 7.65 (d, 2H), 7.61 (d, 2H), 7.52 (d, 2H), 7.42 (br. s., 2H); MS (ESI) m/z 473, 475, 477 [M−1]⁻.

### EXAMPLE 11

### 4-((4-Fluorophenyl)ethynyl)-N-(2-sulfamoylphenylsulfonyl)benzamide

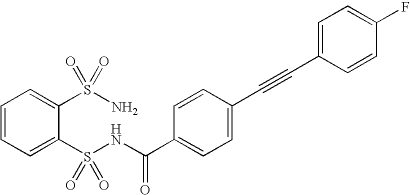

The title compound was synthesized as described for Example 3 in 39% yield, starting from 4-bromo-N-(2-sulfamoylphenylsulfonyl)benzamide and 1-ethynyl-4-fluorobenzene. ¹H NMR (500 MHz, DMSO-d₆) δ ppm 8.35 (d, 1H), 8.15 (d, 1H), 7.83-7.99 (m, 4H), 7.59-7.71 (m, 4H), 7.42 (br. s., 2H), 7.30 (t, 2H); MS (ESI) m/z 457 [M−1]⁻.

### EXAMPLE 12

### 4-((3-Chlorophenyl)ethynyl)-N-(2-sulfamoylphenylsulfonyl)benzamide

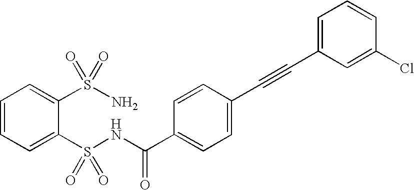

The title compound was synthesized as described for Example 3 in 36% yield, starting from 4-bromo-N-(2-sulfamoylphenylsulfonyl)benzamide and 1-chloro-3-ethynylbenzene. ¹H NMR (500 MHz, DMSO-d₆) δ ppm 8.34 (d, 1H), 8.15 (d, 1H), 7.93 (d, 2H), 7.83-7.91 (m, 2H), 7.63-7.72 (m, 3H), 7.51-7.58 (m, 2H), 7.48 (t, 1H), 7.42 (br. s., 2H); MS (ESI) m/z 473, 475, 477 [M−1]⁻.

### EXAMPLE 13

### 4-((3-Fluorophenyl)ethynyl)-N-(2-sulfamoylphenylsulfonyl)benzamide

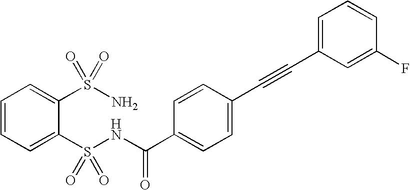

The title compound was synthesized as described for Example 3 in 40% yield, starting from 4-bromo-N-(2-sulfamoylphenylsulfonyl)benzamide and 1-ethynyl-3-fluorobenzene. ¹H NMR (500 MHz, DMSO-d₆) δ ppm 8.29-8.41 (m, 1H), 8.11-8.23 (m, 1H), 7.93 (d, 2H), 7.83-7.91 (m, 2H), 7.67 (d, 2H), 7.38-7.53 (m, 5H), 7.26-7.36 (m, 1H); MS (ESI) m/z 457 [M−1]⁻.

### EXAMPLE 14

### 3-Methoxy-N-(2-sulfamoylphenylsulfonyl)-4-(o-tolylethynyl)benzamide

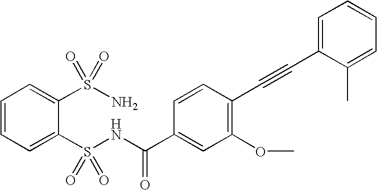

The title compound was synthesized as described for Example 3 in 19% yield, starting from 4-bromo-3-methoxy-N-(2-sulfamoylphenylsulfonyl)benzamide and 1-ethynyl-2-methylbenzene. ¹H NMR (500 MHz, DMSO-d₆) δ ppm 8.35 (d, 1H), 8.16 (d, 1H), 7.83-7.95 (m, 2H), 7.54-7.62 (m, 2H), 7.46-7.53 (m, 2H), 7.43 (br. s., 2H), 7.33 (d, 2H), 7.20-7.28 (m, 1H), 3.92 (s, 3H), 2.47 (s, 3H); MS (ESI) m/z 483 [M−1]⁻.

### EXAMPLE 15

### 4-((4-Chlorophenyl)ethynyl)-3-methoxy-N-(2-sulfamoylphenylsulfonyl)benzamide

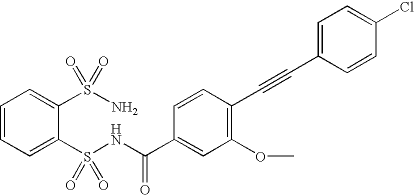

The title compound was synthesized as described for Example 3 in 19% yield, starting from 4-bromo-3-methoxy-N-(2-sulfamoylphenylsulfonyl)benzamide and 1-chloro-4-ethynylbenzene. ¹H NMR (500 MHz, CD₃OD) δ ppm 8.31 (dd, 1H), 8.20 (dd, 1H), 7.72 (d, 1H), 7.64-7.68 (m, 2H), 7.58 (dd, 1H), 7.47-7.51 (m, 2H), 7.41 (d, 1H), 7.36-7.40 (m, 2H), 3.94 (s, 3H); MS (ESI) m/z 503, 505, 507 [M−1]⁻.

### EXAMPLE 16

### 4-((4-Fluorophenyl)ethynyl)-3-methoxy-N-(2-sulfamoylphenylsulfonyl)benzamide

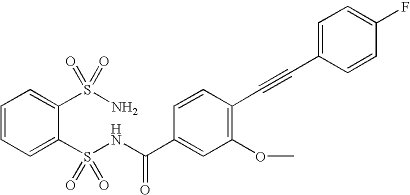

The title compound was synthesized as described for Example 15 in 48% yield, starting from 4-bromo-3-methoxy-N-(2-sulfamoylphenylsulfonyl)benzamide and 1-ethynyl-4-fluorobenzene. ¹H NMR (500 MHz, DMSO-d₆) δ ppm 8.31-8.41 (m, 1H), 8.16 (dd, 1H), 7.82-7.98 (m, 2H), 7.53-7.65 (m, 4H), 7.49 (dd, 1H), 7.43 (s, 2H), 7.24-7.33 (m, 2 H), 3.91 (s, 3H); MS (ESI) m/z 487 [M−1]⁻.

### EXAMPLE 17

### 3-Methoxy-N-(2-sulfamoylphenylsulfonyl)-4-(p-tolylethynyl)benzamide

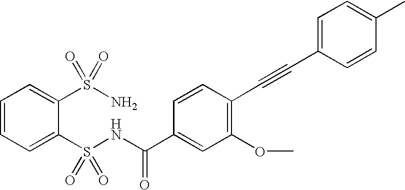

The title compound was synthesized as described for Example 3 in 42% yield, starting from 4-bromo-3-methoxy-N-(2-sulfamoylphenylsulfonyl)benzamide and 1-ethynyl-4-methylbenzene. ¹H NMR (500 MHz, DMSO-d₆) δ ppm 8.31-8.40 (m, 1H), 8.10-8.20 (m, 1H), 7.84-7.95 (m, 2H), 7.58 (d, 1H), 7.52-7.56 (m, 1H), 7.47-7.52 (m, 1H), 7.40-7.46 (m, 4H), 7.25 (d, 2H), 3.91 (s, 3H), 2.34 (s, 3H); MS (ESI) m/z 483 [M−1]⁻.

### EXAMPLE 18

### 3-Methoxy-N-(2-sulfamoylphenylsulfonyl)-4-(m-tolylethynyl)benzamide

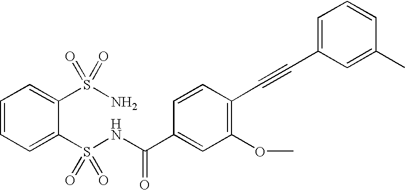

The title compound was synthesized as described for Example 3 in 45% yield, starting from 4-bromo-3-methoxy-N-(2-sulfamoylphenylsulfonyl)benzamide and 1-ethynyl-3-methylbenzene. ¹H NMR (500 MHz, DMSO-d₆) δ ppm 8.32-8.41 (m, 1H), 8.12-8.21 (m, 1H), 7.83-7.98 (m, 2H), 7.59 (d, 1H), 7.53-7.57 (m, 1H), 7.47-7.52 (m, 1H), 7.43 (s, 2H), 7.37 (s, 1H), 7.29-7.35 (m, 2H), 7.25 (d, 1H), 3.91 (s, 3H), 2.32 (s, 3H); MS (ESI) m/z 483 [M−1]⁻.

### EXAMPLE 19

### 4-((3-Chlorophenyl)ethynyl)-3-methoxy-N-(2-sulfamoylphenylsulfonyl)benzamide

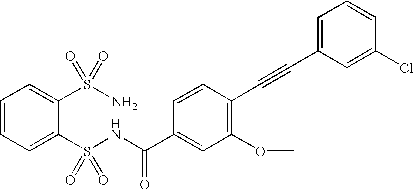

The title compound was synthesized as described for Example 3 in 30% yield, starting from 4-bromo-3-methoxy-N-(2-sulfamoylphenylsulfonyl)benzamide and 1-chloro-3-ethynylbenzene. ¹H NMR (500 MHz, DMSO-d₆) δ ppm 8.36 (d, 1H), 8.13-8.21 (m, 1H), 7.84-7.96 (m, 2H), 7.58-7.63 (m, 3H), 7.46-7.54 (m, 4H), 7.45 (br. s., 2H) 3.92 (s, 3H); MS (ESI) m/z 503, 505, 507 [M−1]⁻.

### a) 4-Bromo-3-methoxy-N-(2-sulfamoylphenylsulfonyl)benzamide

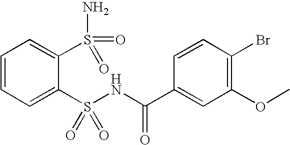

The title compound was synthesized as described for Example 31b) in 80% yield, starting from 4-bromo-3-methoxybenzoic acid. ¹H NMR (400 MHz, DMSO-d₆) δ ppm 8.36 (dd, 1.64 Hz, 1H), 8.17 (dd, 1H), 7.86-7.97 (m, 4H), 7.70 (d, 1H), 7.59 (d, 1H), 7.44 (s, 1H), 7.40 (dd, 1H), 3.90 (s, 3H); MS (ESI) m/z 449, 451 [M+H]⁺

### EXAMPLE 20

### 4-((3-Fluorophenyl)ethynyl)-3-methoxy-N-(2-sulfamoylphenylsulfonyl)benzamide

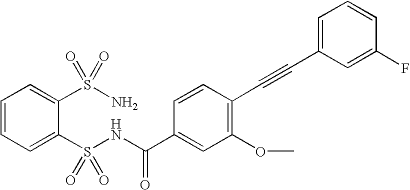

The title compound was synthesized as described for Example 3 in 39% yield, starting from 4-bromo-3-methoxy-N-(2-sulfamoylphenylsulfonyl)benzamide and 1-ethynyl-3-fluorobenzene. ¹H NMR (500 MHz, DMSO-d₆) δ ppm 8.36 (d, 1H), 8.11-8.22 (m, 1H), 7.84-7.98 (m, 2H), 7.56-7.65 (m, 2H), 7.46-7.54 (m, 2H), 7.44 (s, 2H), 7.37-7.42 (m, 2H), 7.26-7.34 (m, 1H), 3.92 (s, 3H); MS (ESI) m/z 487 [M−1]⁻.

### EXAMPLE 21

### 4-(Benzofuran-2-yl)-3-(cyclopropylmethoxy)-N-(2-sulfamoylphenylsulfonyl)benzamide

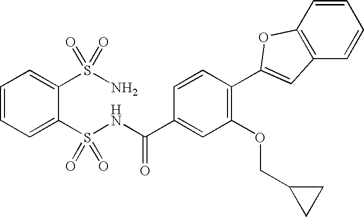

The title compound was synthesized as described for Example 7 in 54% yield, starting from 4-bromo-3-(cyclopropylmethoxy)-N-(2-sulfamoylphenylsulfonyl)benzamide and benzofuran-2-ylboronic acid. ¹H NMR (500 MHz, DMSO-d₆) δ ppm 8.36 (dd, 1H) 8.16 (dd, 1H), 8.03 (d, 1H), 7.84-7.96 (m, 2H), 7.72 (d, 1H), 7.68 (d, 1H), 7.59-7.66 (m, 3 H), 7.43 (s, 2H), 7.36 (td, 1H), 7.24-7.31 (m, 1H), 4.12 (d, 2H), 1.45 (d, 1H), 0.65-0.75 (m, 2H), 0.40-0.50 (m, 2H); MS (ESI) m/z 525 [M−1]⁻.

### 4-Bromo-3-(cyclopropylmethoxy)-N-(2-sulfamoylphenylsulfonyl)benzamide

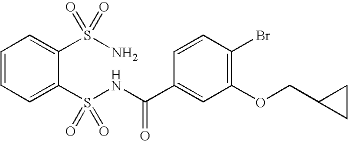

4-Bromo-3-(cyclopropylmethoxy)benzoic acid (0.573 g, 2.11 mmol), N-(3-dimethylaminopropyl)-N′-ethylcarbodiimide hydrochloride (0.608 g, 3.17 mmol) and 4-(dimethylamino)pyridine (0.387 g, 3.17 mmol) were added to a solution of benzene-1,2-disulfonamide (0.499 g, 2.11 mmol) in N,N-dimethylformamide (10 mL) at room temperature, and the resulting mixture was stirred over night. The mixture was concentrated and purified by column chromatography, using ethylacetate followed by ethyl acetate/methanol 100:1 and 10:1 as the eluent, to give 690 mg of the title compound. ¹H NMR (400 MHz, CD₃OD) δ ppm 8.42-8.52 (m, 1H) 8.22-8.32 (m, 1H) 7.78-7.92 (m, 3H) 7.62 (d, 1H) 7.49 (d, 1H) 7.33 (dd, 1H) 3.97 (d, 3H) 1.27-1.40 (m, 2H) 0.56-0.68 (m, 2H) 0.34-0.46 (m, 2H)

### a) 4-Bromo-3-(cyclopropylmethoxy)benzoic acid

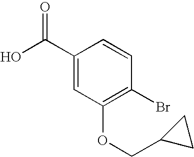

The title compound was synthesized as described for Example 1a) in 89% yield, starting from methyl 4-bromo-3-(cyclopropylmethoxy)benzoate. Workup was performed after stirring over the weekend. ¹H NMR (500 MHz, CD₃OD) δ ppm 7.64 (d, 1H) 7.58 (d, 1H), 7.50 (dd, 1H), 3.97 (d, 2H), 1.26-1.37 (m, 1H), 0.60-0.68 (m, 2H), 0.40-0.46 (m, 2H); MS (ESI) m/z 269, 271 [M−1]⁻.

### b) Methyl 4-bromo-3-(cyclopropylmethoxy)benzoate

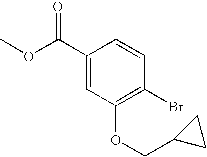

The title compound was synthesized as described for Example 1b), starting from methyl 4-bromo-3-hydroxybenzoate and cyclopropanemethanol. Purification by column chromatography, using heptane/ethyl acetate (10:1) as the eluent, gave the crude title compound. GC MS (EI) m/z 284, 286 [M]^{+•}.

### EXAMPLE 22

### 3-(Cyclopropylmethoxy)-4-(3,3-dimethylbut-1-ynyl)-N-(2-sulfamoylphenylsulfonyl)benzamide

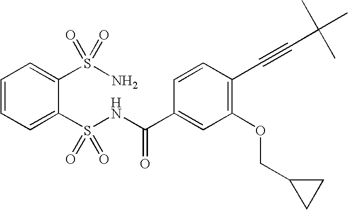

The title compound was synthesized as described for Example 7 in 27% yield, starting from 4-bromo-3-(cyclopropylmethoxy)-N-(2-sulfamoylphenylsulfonyl)benzamide and diisopropyl 3,3-dimethylbut-1-ynylboronate. ¹H NMR (500 MHz, CD₃OD) δ ppm 8.44-8.50 (m, 1H), 8.24-8.29 (m, 1H,) 7.82-7.89 (m, 2H), 7.41 (d, 1H), 7.35-7.39 (m, 1H), 7.30-7.34 (m, 1H), 3.96 (d, 2H), 1.32 (s, 9H), 1.24-1.31 (m, 1H), 0.56-0.63 (m, 2H), 0.41-0.46 (m, 2H); MS (ESI) m/z 489 [M−1]⁻.

### EXAMPLE 23

### 3-(Cyclopropylmethoxy)-4-(3-methylbut-1-ynyl)-N-(2-sulfamoylphenylsulfonyl)benzamide

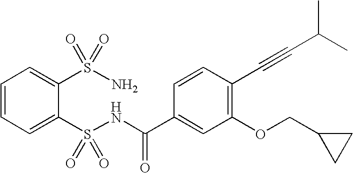

The title compound was synthesized as described for Example 3 in 27% yield, starting from 4-bromo-3-(cyclopropylmethoxy)-N-(2-sulfamoylphenylsulfonyl)benzamide and 3-methylbut-1-yne. ¹H NMR (400 MHz, CD₃OD) δ ppm 8.43-8.53 (m, 1H), 8.22-8.33 (m, 1H), 7.78-7.92 (m, 2H), 7.41 (d, 1H), 7.30-7.38 (m, 2H), 3.94 (d, 2H), 2.74-2.91 (m, 1H), 1.20-1.34 (m, 7H), 0.54-0.66 (m, 2H), 0.36-0.48 (m, 2H); MS (ESI) m/z 475 [M−1]⁻.

### EXAMPLE 24

### N-(2-Sulfamoylphenylsulfonyl)-4-((3,3,4,4-tetrafluoropyrrolidin-1-yl)methyl)benzamide

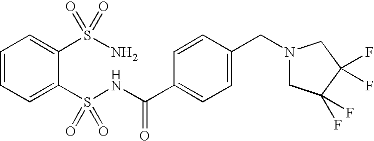

The title compound was synthesized as described for Example 1 in 13% yield, starting from 4-((3,3,4,4-tetrafluoropyrrolidin-1-yl)methyl)benzoic acid. Purification by column chromatography, using ethyl acetate/heptane (10:1) followed by ethyl acetate and a gradient of ethyl acetate/methanol (100:1 to 10:1) as the eluent, followed by purification by preparative HPLC. ¹H NMR (500 MHz, DMSO-d₆) δ ppm 8.35 (dd, 1H), 8.09-8.20 (m, 1H), 7.89 (d, 4H), 7.33-7.47 (m, 4H), 3.77 (s, 2H), 3.16 (t, 4H); MS (ESI) m/z 494 [M−1]⁻.

### a) 4-((3,3,4,4-Tetrafluoropyrrolidin-1-yl)methyl)benzoic acid

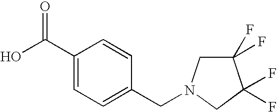

Sodium hydride (60% dispersion in mineral oil, 0.122 g, 3.06 mmol) was added to a solution of 3,3,4,4-tetrafluoropyrrolidine hydrochloride (0.274 g, 1.53 mmol) in N,N-dimethylformamide (4 mL) at room temperature. After 20 min of stirring a solution of methyl 4-(bromomethyl)benzoate (0.350 g, 1.53 mmol) in N,N-dimethylformamide (4 mL) was added to the reaction mixture. After stirring over night the reaction mixture was partitioned between water and ethyl acetate, the organic phase was dried over magnesium sulfate and concentrated. The residue was dissolved in tetrahydrofuran (15 mL) and treated with a solution of lithium hydroxide (0.193 g, 4.59 mmol) in water (4 mL) at room temperature. After stirring over night the reaction mixture was acidified with 2.0 M aqueous hydrochloric acid and partitioned between water and ethyl acetate. The organic phase was dried over magnesium sulfate and the solvent was evaporated to give 0.243 g (90% yield) of the title compound. ¹H NMR (400 MHz, CD₃OD) δ ppm 7.89-8.15 (m, 2H), 7.45 (d, 2H), 3.78 (s, 2H), 2.99-3.21 (m, 4H); MS (ESI) m/z 276 [M−1]⁻.

### EXAMPLE 25

### 3-(3-Hydroxy-3-methylbutoxy)-4-(3-methylbut-1-ynyl)-N-(2-sulfamoylphenylsulfonyl)benzamide

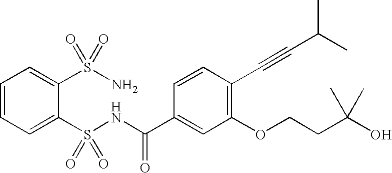

The title compound was synthesized as described for Example 3 in 13% yield, starting from 4-bromo-3-(3-hydroxy-3-methylbutoxy)-N-(2-sulfamoylphenylsulfonyl)benzamide and 3-methylbut-1-yne. ¹H NMR (500 MHz, CD₃OD) δ ppm 8.39-8.49 (m, 1H) 8.21-8.31 (m, 1H), 7.76-7.90 (m, 2H), 7.51 (s, 1H), 7.38-7.44 (m, 1H), 7.33 (d, 1H) 4.22 (t, 2H), 2.80 (m, 1H), 1.97-2.06 (m, 2H), 1.28-1.37 (m, 7H), 1.24-1.28 (m, 3H), 1.24 (s, 3H); MS (ESI) m/z 507 [M−1]⁻.

### a) 4-Bromo-3-(3-hydroxy-3-methylbutoxy)-N-(2-sulfamoylphenylsulfonyl)benzamide

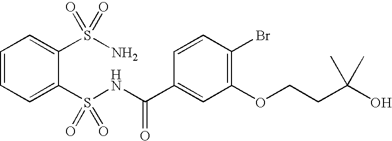

The title compound was synthesized as described for Example 1 in 88% yield, starting from 4-bromo-3-(3-hydroxy-3-methylbutoxy)benzoic acid. Purification by column chromatography using ethyl acetate/methanol (100:1 and 10:1) as the eluent. ¹H NMR (500 MHz, CD₃OD) δ ppm 8.30 (dd, 1H), 8.19 (dd, 1H), 7.61-7.74 (m, 3H), 7.44-7.53 (m, 2H), 4.25 (t, 2H), 2.03 (t, 2H), 1.31 (s, 6H); MS (ESI) m/z 519, 521.

### b) 4-Bromo-3-(3-hydroxy-3-methylbutoxy)benzoic acid

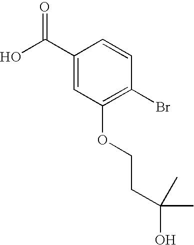

The title compound was synthesized as described for Example 1a) in 93% yield, starting from methyl 4-bromo-3-(3-hydroxy-3-methylbutoxy)benzoate. Workup was performed after stirring over night. ¹H NMR (500 MHz, CD₃OD) δ ppm 7.65 (d, 1H), 7.63 (d, 1H), 7.51 (dd, 1H), 4.26 (t, 2H), 2.05 (t, 2H), 1.32 (s, 6H); MS (ESI) m/z 301, 303.

### c) Methyl 4-bromo-3-(3-hydroxy-3-methylbutoxy)benzoate

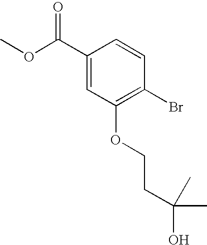

Cesium carbonate (0.423 g, 1.30 mmol) was added to a solution of methyl 4-bromo-3-hydroxybenzoate (0.2 g, 0.87 mmol) and 3-hydroxy-3-methylbutyl 4-methylbenzenesulfonate (0.268 g, 1.04 mmol, Shimizu, Masato et al, Bioorg. Med. Chem., 2006, 14(12), 4277-4294) in N,N-dimethylformamide (10 mL). The reaction mixture was stirred over the weekend, diluted with ethyl acetate and filtered. The filtrate was partitioned between water and ethyl acetate. The organic phase was dried over magnesium sulfate and the solvent was evaporated to give the title compound. ¹H NMR (500 MHz, CDCl₃) δ ppm 7.62 (d, 1H), 7.58 (d, 1H), 7.52-7.56 (m, 1H), 4.32 (t, 2H), 2.10 (t, 2 H), 1.35 (s, 6H); GC MS (EI) m/z 316, 318 [M]⁺.

### EXAMPLE 26

### 3-Isopropoxy-4-(3-methylbut-1-ynyl)-N-(2-sulfamoylphenylsulfonyl)benzamide

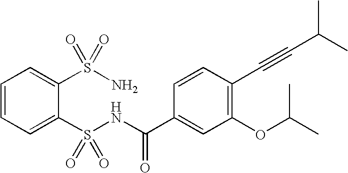

The title compound was synthesized as described for Example 1 in 62% yield, starting from 3-isopropoxy-4-(3-methylbut-1-ynyl)benzoic acid. Purification by preparative HPLC. ¹H NMR (500 MHz, CD₃OD) δ ppm 8.46 (d, 1H), 8.21-8.32 (m, 1H), 7.79-7.91 (m, 2 H), 7.46 (s, 1H), 7.36-7.42 (m, 1H), 7.28-7.36 (m, 1H), 4.68 (dt, 1H), 2.70-2.86 (m, 1H), 1.34 (s, 3H), 1.33 (s, 3H), 1.26 (s, 3H), 1.25 (s, 3H); MS (ESI) m/z 463 [M−1]⁻.

### a) 3-Isopropoxy-4-(3-methylbut-1-ynyl)benzoic acid

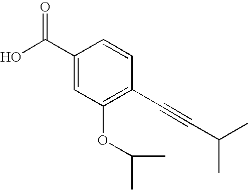

The title compound was synthesized as described for Example 1a) in 99% yield, starting from methyl 3-isopropoxy-4-(3-methylbut-1-ynyl). Workup was performed after stirring over night. ¹H NMR (500 MHz, CD₃OD) δ ppm 7.51-7.57 (m, 2H), 7.32-7.39 (m, 1H), 4.56-4.72 (m, 1H), 2.73-2.90 (m, 1H), 1.33-1.40 (m, 6H), 1.22-1.32 (m, 6H); MS (ESI) m/z 245 [M−1]⁻.

### b) Methyl 3-isopropoxy-4-(3-methylbut-1-ynyl)benzoate

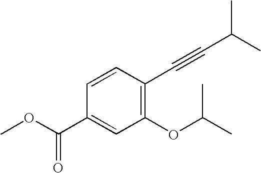

The title compound was synthesized as described for Example 3 in 21% yield, starting from methyl 4-bromo-3-isopropoxybenzoate (0.226 g, 0.83 mmol) and 3-methylbut-1-yne. Purification by column chromatography, using a gradient of heptane/ethyl acetate (10:1 to 8:1) as the eluent, followed by purification by preparative HPLC. GC MS (EI) m/z 260 [M]^{+•}.

### EXAMPLE 27

### 4-(Cyclopropylethynyl)-3-(3-methoxy-3-methylbutoxy)-N-(2-sulfamoylphenylsulfonyl)benzamide

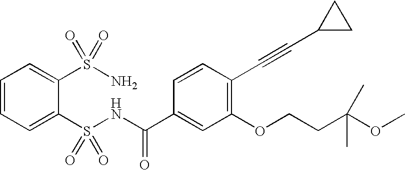

The title compound was synthesized as described for Example 3 in 29% yield, starting from 4-bromo-3-(3-methoxy-3-methylbutoxy)-N-(2-sulfamoylphenylsulfonyl)benzamide and ethynylcyclopropane. ¹H NMR (500 MHz, CD₃OD) δ ppm 8.44-8.51 (m, 1H), 8.22-8.32 (m, 1H), 7.80-7.91 (m, 2H), 7.46 (d, 1H), 7.30-7.38 (m, 2H), 4.15 (t, 2H), 3.25 (s, 3H), 2.04 (t, 2H), 1.45-1.54 (m, 1H), 1.29 (s, 6H), 0.86-0.95 (m, 2H), 0.69-0.77 (m, 2H); MS (ESI) m/z 519 [M−1]⁻.

### a) 4-Bromo-3-(3-methoxy-3-methylbutoxy)-N-(2-sulfamoylphenylsulfonyl)-benzamide

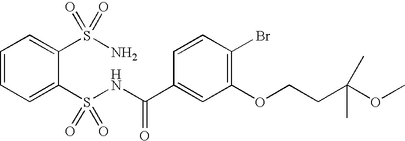

The title compound was synthesized as described for Example 1 in 75% yield, starting from 4-bromo-3-(3-methoxy-3-methylbutoxy)benzoic acid. Purification by column chromatography, using ethyl acetate/heptane (10:1) followed by ethyl acetate as the eluent. ¹H NMR (500 MHz, CD₃OD) δ ppm 8.33 (d, 1H), 8.21 (dd, 1H), 7.64-7.77 (m, 3H), 7.52 (d, 1H), 7.44 (dd, 1H), 4.19 (t, 2H), 3.24 (s, 3H), 2.06 (t, 2H), 1.22-1.35 (m, 6H); MS (ESI) m/z 533, 535 [M−1]⁻.

### b) 4-Bromo-3-(3-methoxy-3-methylbutoxy)benzoic acid

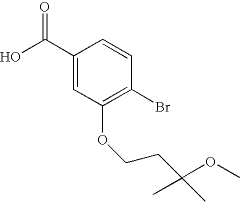

The title compound was synthesized as described for Example 1a) in 99% yield, starting from methyl 4-bromo-3-(3-methoxy-3-methylbutoxy)benzoate. Workup was performed after stirring over night. ¹H NMR (500 MHz, CDCl₃) δ ppm 7.69 (d, 1H), 7.64 (d, 1H), 7.57 (dd, 1H), 4.25 (t, 2H), 3.27 (s, 3H), 2.13 (t, 2H), 1.31 (s, 6H); MS (ESI) m/z 315, 317 [M−1]⁻.

### c) Methyl 4-bromo-3-(3-methoxy-3-methylbutoxy)benzoate

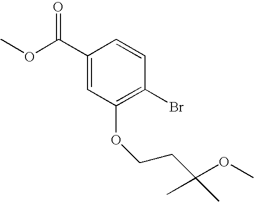

The title compound was synthesized as described for Example 1b) in 98% yield, starting from methyl 4-bromo-3-hydroxybenzoate and 3-methoxy-3-methyl-1-butanol. ¹H NMR (500 MHz, CDCl₃) δ ppm 7.61 (d, 1H), 7.56 (d, 1H), 7.50 (dd, 1H), 4.19 (t, 2H), 3.93 (s, 3H), 3.25 (s, 3H), 2.10 (t, 2H), 1.29 (s, 6H); GC MS (EI) m/z 330, 332 [M]^{+•.}

### EXAMPLE 28

### 3-Isobutoxy-4-(3-methylbut-1-ynyl)-N-(2-sulfamoylphenylsulfonyl)benzamide

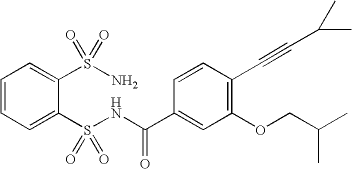

The title compound was synthesized as described for Example 3 in 15% yield, starting from 4-bromo-3-isobutoxy-N-(2-sulfamoylphenylsulfonyl)benzamide and 3-methylbut-1-yne. The reaction was heated at 65° C. for 4 days. ¹H NMR (500 MHz, CD₃OD) δ ppm 8.41-8.51 (m, 1H), 8.20-8.32 (m, 1H), 7.79-7.91 (m, 2H), 7.45 (s, 1H), 7.35-7.41 (m, 1H), 7.30-7.35 (m, 1H), 3.84 (d, 2H), 2.81 (dt, 1H), 2.10 (dt, 1H), 1.25 (d, 6H), 1.08 (d, 6H); MS (ESI) m/z 477 [M−1]⁻.

### a) 4-Bromo-3-isobutoxy-N-(2-sulfamoylphenylsulfonyl)benzamide

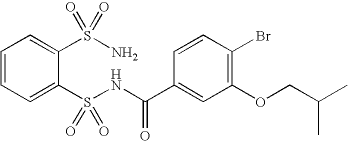

The title compound was synthesized as described for Example 1 in 73% yield, starting from 4-bromo-3-isobutoxybenzoic. Purification by column chromatography, using ethyl acetate/heptane (10:1) followed by ethyl acetate as the eluent. ¹H NMR (500 MHz, CD₃OD) δ ppm 8.29-8.37 (m, 1H), 8.20-8.29 (m, 1H), 7.68-7.77 (m, 2H), 7.66 (d, 1H), 7.51-7.57 (m, 1H), 7.44-7.51 (m, 1H), 3.87 (d, 2H), 2.05-2.21 (m, 1H), 1.10 (s, 3H), 1.08 (s, 3H); MS (ESI) m/z 489, 491 [M−1]⁻.

### b) 4-Bromo-3-isobutoxybenzoic acid

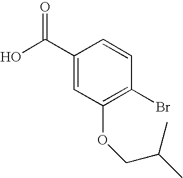

The title compound was synthesized as described for Example 1a) in 98% yield, starting from methyl 4-bromo-3-isobutoxybenzoate. Workup was performed after stirring over night. ¹H NMR (500 MHz, CDCl₃) δ ppm 7.65 (d, 1H), 7.55-7.60 (m, 2H), 3.87 (d, 2H), 2.13-2.27 (m, 1H), 1.11 (s, 3H), 1.09 (s, 3H); MS (ESI) m/z 271, 273 [M−1]⁻.

### c) Methyl 4-bromo-3-isobutoxybenzoate

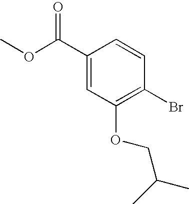

The title compound was synthesized as described for Example 1b) in 94% yield, starting from methyl 4-bromo-3-hydroxybenzoate and 2-methyl-1-propanol. ¹H NMR (500 MHz, CDCl₃) δ ppm 7.61 (d, 1H), 7.51 (d, 1H), 7.47-7.51 (m, 1H), 3.92 (s, 3H), 3.85 (d, 2H), 2.14-2.23 (m, 1H), 1.10 (s, 3H), 1.08 (s, 3H); GC MS (EI) m/z 286, 288 [M]^{+•}.

### EXAMPLE 29

### 3-Methoxy-4-(4-methylbenzyloxy)-N-(2-sulfamoylphenylsulfonyl)benzamide

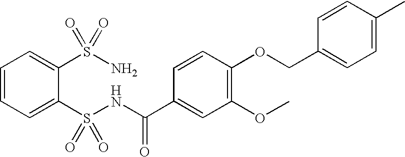

The title compound was synthesized as described for Example 1 in 50% yield, starting from 3-methoxy-4-(4-methylbenzyloxy)benzoic acid. ¹H NMR (400 MHz, DMSO-d₆) ppm 8.30-8.38 (m, 1H), 8.10-8.20 (m, 1H), 7.83-7.97 (m, 2H), 7.53 (dd, 1H), 7.49 (d, 1H), 7.40 (s, 2H), 7.29-7.35 (m, 2H), 7.16-7.23 (m, 2H), 7.11 (d, 1H), 5.11 (s, 2 H), 3.78 (s, 3H), 2.30 (s, 3H); MS (ESI) m/z 489 [M−1]⁻.

### a) 3-Methoxy-4-(4-methylbenzyloxy)benzoic acid

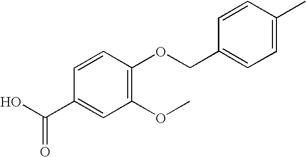

The title compound was synthesized as described for Example 1a) in 85% yield, starting from methyl 3-methoxy-4-(4-methylbenzyloxy)benzoate. Workup was performed after stirring over night. ¹H NMR (400 MHz, DMSO-d₆) δ ppm 7.53 (dd, 1H), 7.45 (d, 1H), 7.33 (d, 2H), 7.20 (d, 2H), 7.12 (d, 1H), 5.11 (s, 2H), 3.79 (s, 3H), 2.31 (s, 3H); MS (ESI) m/z 271 [M−1]⁻.

### b) Methyl 3-methoxy-4-(4-methylbenzyloxy)benzoate

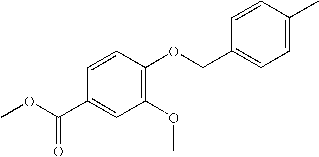

alpha-Bromo-p-xylene (0.335 g, 1.81 mmol) and potassium carbonate (0.455 g, 3.29 mmol) were added to a solution of methyl 4-hydroxy-3-methoxybenzoate (0.3 g, 1.65 mmol) in acetone (20 mL). The reaction mixture was stirred over night at room temperature, filtered through a plug of celite and the filtrate was evaporated. The residue was dissolved in ethyl acetate and washed with brine. The organic phase was dried over magnesium sulfate and the solvent was evaporated to give the crude title compound. GC MS (EI) m/z 286 [M]^{+•}.

### EXAMPLE 30

### 4-(Benzyloxy)-N-(2-sulfamoylphenylsulfonyl)-3-(3,3,3-trifluoropropoxy)benzamide

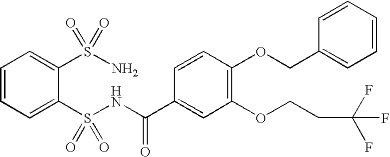

The title compound was synthesized as described for Example 1 in 14% yield, starting from 4-(benzyloxy)-3-(3,3,3-trifluoropropoxy)benzoic acid. ¹H NMR (400 MHz, DMSO-d₆) δ ppm 8.30-8.39 (m, 1H), 8.10-8.20 (m, 1H), 7.83-7.99 (m, 2H), 7.57 (d, 1H), 7.51-7.56 (m, 1H), 7.29-7.47 (m, 7H), 7.14 (d, 1H), 5.20 (s, 2H), 4.26 (t, 2H), 2.72-2.91 (m, 2H); MS (ESI) m/z 557 [M−1]⁻.

### a) 4-(Benzyloxy)-3-(3,3,3-trifluoropropoxy)benzoic acid

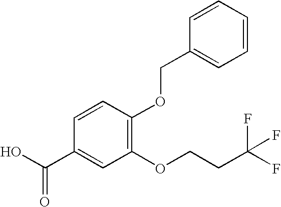

The title compound was synthesized as described for Example 1a) in 90% yield, starting from methyl 4-(benzyloxy)-3-(3,3,3-trifluoropropoxy)benzoate. After stirring over night an additional equivalent of lithium hydroxide was added and the reaction mixture was stirred for 5 days before workup was performed. MS (ESI) m/z 339 [M−1]⁻.

### b) Methyl 4-(benzyloxy)-3-(3,3,3-trifluoropropoxy)benzoate

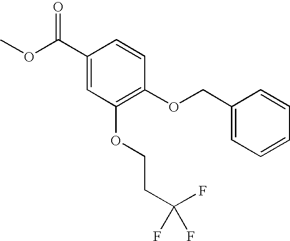

Diisopropyl azodicarboxylate (0.372 mL, 1.89 mmol) and triphenylphosphine (0.489 g, 1.86 mmol) were added to a solution of methyl 4-(benzyloxy)-3-hydroxybenzoate (0.355 g, 1.37 mmol) in tetrahydrofuran (20 mL) and the resulting reaction mixture was stirred for 55 min at room temperature. 3,3,3-Trifluoro-1-propanol (0.121 mL, 1.37 mmol) was added and the reaction mixture was stirred over night. The solvent was evaporated. Purification by column chromatography, using heptane/ethyl acetate (6:1) as the eluent, gave 0.162 g (33% yield) of the title compound. GC MS (EI) m/z 354 [M]^{+•}.

### EXAMPLE 31

### 4-(3,3-Dicyclopropyl-3-hydroxyprop-1-ynyl)-N-(2-sulfamoylphenylsulfonyl)benzamide

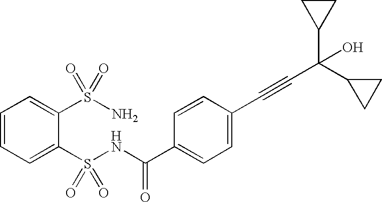

Copper(I) iodide (0.753 μL, 0.02 mmol) was added to a stirred solution of 4-iodo-N-(2-sulfamoylphenylsulfonyl)benzamide (0.1036 g, 0.22 mmol), 1,1-dicyclopropylprop-2-yn-1-ol (0.034 g, 0.25 mmol), tetrakis(triphenylphosphine)palladium(0) (0.013 g, 0.01 mmol) and triethylamine (0.25 mL, 1.79 mmol) in anhydrous N,N-dimethylformamide (3 mL) under an atmosphere of nitrogen. The resulting mixture was heated at 65° C. over night. Water and ethyl acetate was added and the phases were separated. The aqueous phase was acidified (pH ˜1) with hydrochloric acid (2 M) and extracted with ethyl acetate. The combined organic phases were washed with water, water/brine (1:1) and brine, dried over magnesium sulfate and the solvent was evaporated. Purification by preparative HPLC gave 0.042 g (40% yield) of the title compound. ¹HNMR (500 MHz, DMSO-d₆) δ ppm 8.26-8.34 (m, 1H) 8.08-8.16 (m, 1H) 7.80-7.86 (m, 2H) 7.86 (d, 2H), 7.37-7.47 (m, 3H) 1.13-1.24 (m, 2H) 0.51-0.60 (m, 2H) 0.35-0.44 (m, 4H); MS (APCI) m/z 473.1 [M−1]⁻.

### a) 1,1-Dicyclopropylprop-2-yn-1-ol

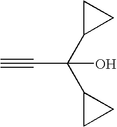

Dicyclopropylmethanone (0.5 mL, 4.39 mmol) was added dropwise over 5 min to cooled (0° C.) ethynylmagnesium bromide (0.5 M in tetrahydrofuran, 10 mL, 5.00 mmol) and the mixture was stirred at 0° C. for 2 h and at room temperature over night. Saturated ammonium chloride was added followed by diethyl ether and the mixture was acidified (pH ˜1) with hydrochloric acid (2 M). The aqueous phase was extracted with diethyl ether, the combined organic phases were washed with water and brine, dried over magnesium sulfate and the solvent was evaporated to give 0.572 g (96% yield) of the title compound. ¹H NMR (500 MHz, CDCl₃) δ ppm 2.31 (s, 1H) 2.02 (s, 1H) 1.21-1.30 (m, 2H) 0.60-0.68 (m, 2H) 0.51-0.58 (m, 4H) 0.44-0.50 (m, 2H); GC MS (EI) m/z 135 [M]^{+•}.

### b) 4-Iodo-N-(2-sulfamoylphenylsulfonyl)benzamide

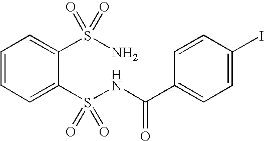

1-(3-Dimethylaminopropyl)-3-ethylcarbodiimide hydrochloride (1.2024 g, 6.27 mmol) was added to a stirred solution of benzene-1,2-disulfonamide (1.0045 g, 4.25 mmol), 4-iodobenzoic acid (1.0665 g, 4.30 mmol) and 4-dimethylaminopyridine (0.7765 g, 6.36 mmol) in anhydrous N,N-dimethylformamide (25 mL) and the resulting mixture was stirred at room temperature for 5 h. Water and ethyl acetate was added and the phases were separated. The aqueous phase was acidified (pH˜1) with hydrochloric acid (2 M) and extracted with ethyl acetate. The combined organic phases were washed with water, water/brine (1:1) and brine, dried over magnesium sulfate and the solvent was evaporated to give 1.833 g (92% yield) of the title compound. ¹H NMR (500 MHz, DMSO-d₆) δ ppm 8.28-8.37 (m, 1H) 8.12-8.19 (m, 1H) 7.86-7.93 (m, 2H) 7.87 (d, 2H) 7.64 (d, 2H) 7.41 (br. s., 2H); MS (ESI) m/z 465.0 [M−1]⁻.

### EXAMPLE 32

### 3-(Hydroxymethyl)-4-(3-methylhex-1-ynyl)-N-(2-sulfamoylphenylsulfonyl)benzamide

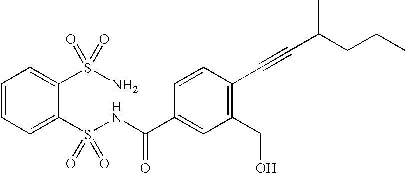

The title compound was synthesized as described for Example 31 in 11% yield, starting from 4-bromo-3-(hydroxymethyl)-N-(2-sulfamoylphenylsulfonyl)benzamide and 3-methylhex-1-yne. ¹H NMR (500 MHz, DMSO-d₆) δ ppm 8.25-8.37 (m, 1H) 8.09-8.19 (m, 1H) 7.95-8.03 (m, 1H) 7.86 (br. s., 2H) 7.72-7.78 (m, 1H) 7.33-7.44 (m, 3H) 4.60 (s, 2H) 2.70-2.79 (m, 1H) 1.37-1.55 (m, 5H) 1.22 (d, 3H) 0.92 (t, 3H); MS (APCI) m/z 463.1 [M−1]⁻.

### a) 4-Bromo-3-(hydroxymethyl)-N-(2-sulfamoylphenylsulfonyl)benzamide

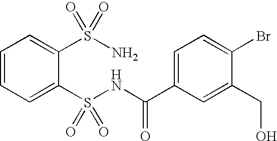

1-(3-Dimethylaminopropyl)-3-ethylcarbodiimide hydrochloride (3.86 g, 20.13 mmol) was added to a stirred solution of benzene-1,2-disulfonamide (3.17 g, 13.42 mmol), 4-bromo-3-(hydroxymethyl)benzoic acid (3.1 g, 13.42 mmol) and 4-dimethylaminopyridine (2.459 g, 20.13 mmol) in N,N-dimethylformamide (30 mL) and the resulting mixture was stirred at room temperature over night. Water and ethyl acetate was added and the phases were separated. The aqueous phase was acidified (pH˜1) with hydrochloric acid (2 M) and extracted with ethyl acetate. The combined organic phases were washed with water, water/brine (1:1) and brine, dried over magnesium sulfate and the solvent was evaporated. Purification by column chromatography, using a gradient of 0-20% methanol in dichloromethane as the eluent. Saturated aqueous sodium bicarbonate and ethyl acetate were added and the aqueous phase was washed with ethyl acetate, acidified using hydrochloric acid (5 M) and extracted with ethyl acetate. The organic phase was washed with water, dried over magnesium sulfate and the solvent was evaporated to give 2.472 g (41% yield) of the title compound. ¹H NMR (500 MHz, DMSO-d₆) δ ppm 9.67 (br. s., 1H) 8.08-8.13 (m, 2H) 7.96-8.01 (m, 1H) 7.64-7.68 (m, 1H) 7.60-7.64 (m, 1H) 7.55-7.60 (m, 1H) 7.51 (s, 1H) 7.44 (br. s., 2H) 5.40-5.50 (m, 1H) 4.49 (d, 2H); MS (ESI) m/z 447, 449 [M−1]⁻.

### b) 4-bromo-3-(hydroxymethyl)benzoic acid

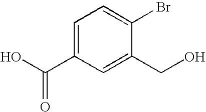

A solution of lithium hydroxide monohydrate (2.278 g, 54.3 mmol) in water (12 mL) was added to a solution of methyl 3-(acetoxymethyl)-4-bromobenzoate (3.89 g, 13.55 mmol) in tetrahydrofuran (40 mL) and the mixture was stirred at room temperature over night. The pH was set to ˜1 with hydrochloric acid (2 M). Water and ethyl acetate was added and the aqueous phase was extracted with ethyl acetate. The combined organic phases were washed with water and brine, dried over magnesium sulfate and concentrated to give 3.06 g (98% yield) of the title compound. ¹H NMR (500 MHz, DMSO-d₆) δ ppm 13.15 (br. s., 1H) 8.11 (d, 1H) 7.65-7.76 (m, 2H) 5.55-5.69 (m, 1H) 4.54 (d, 2H); MS (ESI) m/z 229, 231 [M−1]⁻.

### c) Methyl 3-(acetoxymethyl)-4-bromobenzoate

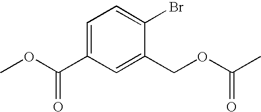

Potassium acetate (4.3253 g, 44.07 mmol) was added to a mixture of methyl 4-bromo-3-(bromomethyl)benzoate (6.63 g, 21.53 mmol) in acetic acid (26 mL) and the mixture was heated at 100° C. for 5 h and at room temperature over night. Water and ethyl acetate was added. The aqueous phase was extracted with ethyl acetate and the combined organic phases were washed with water, saturated sodium hydrogen carbonate, water and brine, dried over magnesium sulfate and the solvent was evaporated. The residue was purified by column chromatography, using a gradient of 0-30% ethyl acetate in n-heptane as the eluent, to give 3.89 g (63% yield from methyl 4-bromo-3-methylbenzoate) of the title compound. ¹H NMR (500 MHz, CDCl₃) δ ppm 8.07 (d, 1H) 7.86 (dd, 1H) 7.67 (d, 1H) 5.22 (s, 2H) 3.94 (s, 3H) 2.18 (s, 3H).

### d) Methyl 4-bromo-3-(bromomethyl)benzoate

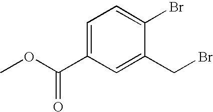

N-Bromosuccinimide (1.947 mL, 22.95 mmol) and 2,2′-azobisisobutyronitrile (0.0225 g, 0.14 mmol) was added to a stirred solution of methyl 4-bromo-3-methylbenzoate (5.0519 g, 22.05 mmol) in carbon tetrachloride (70 mL) and the resulting mixture was stirred at 70° C. for 2 days and at 80° C. for 6 h. Water and chloroform was added. The aqueous phase was extracted with chloroform and the combined organic phases were washed with water and saturated aqueous sodium hydrogen carbonate, dried over magnesium sulfate and the solvent was evaporated to give 6.63 g of the title compound used in the next step without further purification. ¹H NMR (500 MHz, CDCl₃) δ ppm 8.13 (d, 1H) 7.83 (dd, 1H) 7.68 (d, 1H) 4.63 (s, 2H) 3.94 (s, 3H); GC, ms detection: GC MS (EI) m/z 308 [M]^{+•}.

### EXAMPLE 33

### 3-Isopropoxy-N-(2-sulfamoylphenylsulfonyl)-4-((4-(trifluoromethyl)phenyl)ethynyl)benzamide

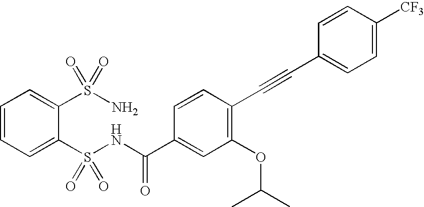

The title compound was synthesized as described for Example 31 in 13% yield, starting from 4-bromo-3-isopropoxy-N-(2-sulfamoylphenylsulfonyl)benzamide and 1-ethynyl-4-(trifluoromethyl)benzene. After purification by preparative HPLC dichloromethane was added followed by n-heptane until a milky solution was obtained. The solid was removed by filtration and dried in vacuo. ¹H NMR (500 MHz, DMSO-d₆) δ ppm 8.28-8.40 (m, 1H) 8.08-8.19 (m, 1H) 7.83-7.92 (m, 2H) 7.78-7.83 (m, 2H) 7.70-7.76 (m, 2H) 7.60-7.63 (m, 1H) 7.54-7.60 (m, 1H) 7.42-7.50 (m, 3H) 4.71-4.84 (m, 1H) 1.35 (d, 6H); MS (APCI) m/z 565.1 [M−1]⁻.

### a) 4-Bromo-3-isopropoxy-N-(2-sulfamoylphenylsulfonyl)benzamide

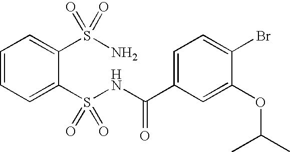

1-(3-Dimethylaminopropyl)-3-ethylcarbodiimide hydrochloride (0.571 g, 2.98 mmol) was added to a stirred solution of benzene-1,2-disulfonamide (0.726 g, 3.07 mmol), 4-bromo-3-isopropoxybenzoic acid (0.500 g, 1.93 mmol) and 4-dimethylaminopyridine (0.3479 g, 2.85 mmol) in N,N-dimethylformamide (30 mL) and the resulting mixture was stirred at room temperature over night. Water and ethyl acetate was added and the phases were separated. The aqueous phase was acidified (pH˜1) with hydrochloric acid (2 M) and extracted with ethyl acetate. The combined organic phases were washed with water, water/brine (1:1) and brine, dried over magnesium sulfate and the solvent was evaporated to give 1.089 g (118% yield) of the title compound. MS (ESI) m/z 476.9 [M−1]⁻.

### b) 4-Bromo-3-isopropoxybenzoic acid

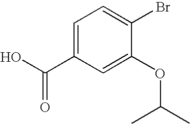

Isopropyl alcohol (0.26 mL, 3.40 mmol) was added to a stirred solution of diisopropyl azodicarboxylate (0.67 mL, 3.40 mmol), triphenylphosphine (0.743 mL, 3.40 mmol) and methyl 4-bromo-3-hydroxybenzoate (0.5239 g, 2.27 mmol) in tetrahydrofuran (30 mL) and the mixture was stirred at room temperature over night. A solution of lithium hydroxide monohydrate (0.2883 g, 6.87 mmol) in water (2 mL) was added and the resulting mixture was stirred over night. Additional lithium hydroxide monohydrate (0.3123 g, 7.44 mmol) in water (2 mL) was added and the mixture was stirred at room temperature for 6 days. Water and ethyl acetate was added and the aqueous phase was washed with ethyl acetate. The aqueous phase was acidified (pH˜1) using hydrochloric acid (2 M) and extracted with ethyl acetate. The combined organic phases were washed with water and brine, dried over magnesium sulfate and the solvent was evaporated to give 0.505 g (86% yield) of the title compound. MS (ESI) m/z 257, 259 [M−1]⁻.

### EXAMPLE 34

### 4-(3-Methylhex-1-ynyl)-N-(2-sulfamoylphenylsulfonyl)-3-(2-(2,2,2-trifluoroethoxy)ethoxy)benzamide

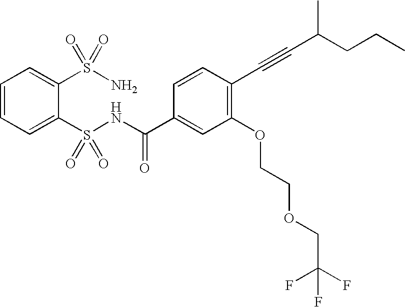

The title compound was synthesized as described for Example 31 in 34% yield, starting from 4-bromo-N-(2-sulfamoylphenylsulfonyl)-3-(2-(2,2,2-trifluoroethoxy)ethoxy)benzamide and 3-methylhex-1-yne. The reaction mixture was heated at 65° C. for 1.5 weeks. ¹H NMR (600 MHz, DMSO-d₆) δ pm 8.34 (d, 1H) 8.14 (d, 1H) 7.84-7.93 (m, 2H) 7.53 (s, 1H) 7.40-7.45 (m, 3H) 7.36-7.39 (m, 1H) 4.21-4.26 (m, 2H) 4.20 (q, 2H) 3.96-4.02 (m, 2H) 2.66-2.75 (m, 1H) 1.48-1.56 (m, 1H) 1.40-1.47 (m, 3H) 1.19 (d, 3H) 0.90 (t, 3H), MS (APCI) m/z 575.2 [M−1]⁻, MS (APCI) m/z 577.2 [M+1]⁺.

### a) 4-Bromo-N-(2-sulfamoylphenylsulfonyl)-3-(2-(2,2,2-trifluoroethoxy)ethoxy)benzamide

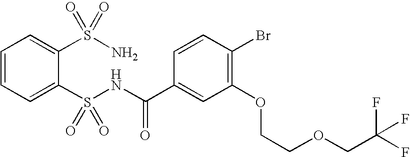

1-(3-Dimethylaminopropyl)-3-ethylcarbodiimide hydrochloride (0.5098 g, 2.66 mmol) was added to a stirred solution of benzene-1,2-disulfonamide (0.4273 g, 1.81 mmol), 4-bromo-3-(2-(2,2,2-trifluoroethoxy)ethoxy)benzoic acid (0.618 g, 1.80 mmol) and 4-dimethylaminopyridine (0.3313 g, 2.71 mmol) in anhydrous N,N-dimethylformamide (12 mL) and the resulting mixture was stirred at room temperature over night. Water and ethyl acetate was added and the aqueous phase was acidified (pH˜1) with hydrochloric acid (2 M) and extracted with ethyl acetate. The combined organic phases were washed with water, water/brine (1:1) and brine, dried over magnesium sulfate and the solvent was evaporated to give 0.987 g (98% yield) of the title compound. ¹H NMR (600 MHz, DMSO-d₆) δ ppm 8.35 (d, 1H) 8.15 (d, 1H) 7.86-7.93 (m, 2H) 7.70 (d, 1H) 7.61 (d, 1H) 7.43 (s, 2H) 7.39 (dd, 1H) 4.27-4.32 (m, 2H) 4.20 (q, 2H) 3.98-4.02 (m, 2H); MS (ESI) m/z 559, 561 [M−1]⁻.

### b) 4-Bromo-3-(2-(2,2,2-trifluoroethoxy)ethoxy)benzoic acid

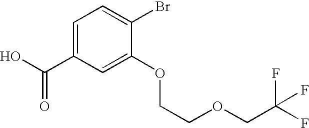

A solution of lithium hydroxide monohydrate (0.2448 g, 5.83 mmol) in water (3 mL) was added to a solution of methyl 4-bromo-3-(2-(2,2,2-trifluoroethoxy)ethoxy)benzoate (0.720 g, 2.02 mmol) in tetrahydrofuran (10 mL) and the resulting mixture was stirred at room temperature over the weekend. Water and ethyl acetate was added and the aqueous phase was acidified (pH˜1) with hydrochloric acid (2 M) and extracted with ethyl acetate. The combined organic phases were washed with water and brine, dried over magnesium sulfate and the solvent was evaporated to give 0.627 g (91% yield) of the title compound. ¹H NMR (600 MHz, DMSO-d₆) δ ppm 13.24 (br. s., 1H) 7.72 (d, 1H) 7.56 (d, 1H) 7.47 (dd, 1H) 4.27-4.32 (m, 2H) 4.21 (q, 2H) 3.96-4.01 (m, 2H); MS (ESI) m/z 341, 343 [M−1]⁻.

### c) Methyl 4-bromo-3-(2-(2,2,2-trifluoroethoxy)ethoxy)benzoate

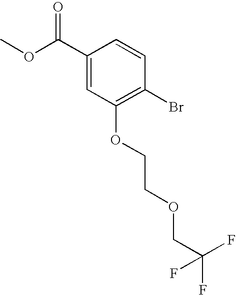

Diisopropyl azodicarboxylate (0.647 mL, 3.28 mmol) was added to a stirred solution of methyl 4-bromo-3-hydroxybenzoate (0.5058 g, 2.19 mmol), triphenylphosphine (0.861 g, 3.28 mmol) and 2-(2,2,2-trifluoroethoxy)ethanol (0.367 mL, 3.28 mmol) in tetrahydrofuran (10 mL) and the resulting mixture was stirred at room temperature over night. Water and ethyl acetate was added and the aqueous phase was extracted with ethyl acetate. The combined organic phases were washed with water, water/brine (1:1) and brine, dried over to magnesium sulfate and the solvent was evaporated. Purification by column chromatography, using a gradient of methanol (0-10%) in dichloromethane as the eluent, gave 0.737 g (94% yield) of the title compound. ¹H NMR (600 MHz, DMSO-d₆) δ ppm 7.76 (d, 1H) 7.57 (d, 1H) 7.48 (dd, 1H) 4.28-4.33 (m, 2H) 4.20 (q, 2H) 3.97-4.02 (m, 2H) 3.86 (s, 3H); GC MS (EI) m/z 356, 358 [M]⁺.

### EXAMPLE 35

### 4-(3-Methylhex-1-ynyl)-N-(2-sulfamoylphenylsulfonyl)-3-(3,3,3-trifluoropropoxy)benzamide

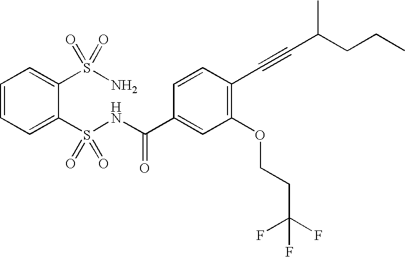

The title compound was synthesized as described for Example 31 in 20% yield, starting from 4-bromo-N-(2-sulfamoylphenylsulfonyl)-3-(3,3,3-trifluoropropoxy)benzamide and 3-methylhex-1-yne. The reaction mixture was heated at 60° C. over the weekend. ¹H NMR (600 MHz, DMSO-d₆) δ ppm 8.27-8.36 (m, 1H) 8.09-8.16 (m, 1H) 7.76-7.93 (m, 2H) 7.55 (s, 1H) 7.39-7.47 (m, 3H) 7.33-7.38 (m, 1H) 4.28 (t, 2H) 2.75-2.87 (m, 2H) 2.66-2.73 (m, 1H) 1.49-1.59 (m, 1H) 1.37-1.49 (m, 3H) 1.18 (d, 3H) 0.90 (t, 3H); MS (APCI) m/z 545.2 [M−1]⁻.

### a) 4-Bromo-N-(2-sulfamoylphenylsulfonyl)-3-(3,3,3-trifluoropropoxy)benzamide

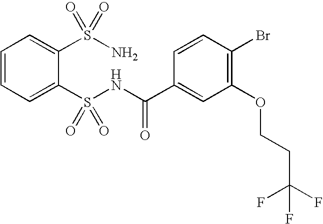

1-(3-Dimethylaminopropyl)-3-ethylcarbodiimide hydrochloride (0.1564 g, 0.82 mmol) was added to a stirred solution of benzene-1,2-disulfonamide (0.1297 g, 0.55 mmol), 4-bromo-3-(3,3,3-trifluoropropoxy)benzoic acid (0.170 g, 0.54 mmol) and 4-dimethylaminopyridine (0.1005 g, 0.82 mmol) in anhydrous N,N-dimethylformamide (4 mL) and the resulting mixture was stirred at room temperature overnight. Water and ethyl acetate was added. The aqueous phase was acidified (pH˜1) with hydrochloric acid (2 M) and extracted with ethyl acetate. The combined organic phases were washed with water, water/brine (1:1) and brine, dried over magnesium sulfate and the solvent was evaporated. Purification by preparative HPLC gave 0.191 g (66% yield) of the title compound. ¹H NMR (600 MHz, DMSO-d₆) δ ppm 8.35 (d, 1H) 8.15 (dd, 1H) 7.84-7.94 (m, 2H) 7.70 (d, 1H) 7.63 (d, 1H) 7.43 (s, 2H) 7.39 (dd, 1H) 4.36 (t, 2H) 2.87 (ddd, 2H); MS (ESI) m/z 529, 531 [M−1]⁻

### b) 4-Bromo-3-(3,3,3-trifluoropropoxy)benzoic acid

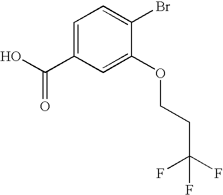

A solution of lithium hydroxide monohydrate (0.0840 g, 2.00 mmol) in water (1 mL) was added to a solution of methyl 4-bromo-3-(3,3,3-trifluoropropoxy)benzoate (0.215 g, 0.66 mmol) in tetrahydrofuran (3.5 mL) and the resulting mixture was stirred at room temperature for 3 h. Water and ethyl acetate was added and the aqueous phase was acidified (pH˜1) with hydrochloric acid (2 M). The aqueous phase was extracted with ethyl acetate, the combined organic phases were dried over magnesium sulfate and the solvent was evaporated to give 0.179 g (87% yield) of the title compound. ¹H NMR (600 MHz, DMSO-d₆) δ ppm 13.24 (br. s., 1H) 7.73 (d, 1H) 7.57 (d, 1H) 7.48 (dd, 1H) 4.36 (t, 2H) 2.83 (dt, 2H); MS (ESI) m/z 311, 313 [M−1]⁻.

### c) Methyl 4-bromo-3-(3,3,3-trifluoropropoxy)benzoate

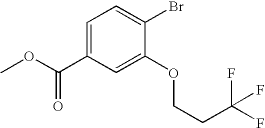

3,3,3-Trifluoro-1-propanol (0.294 mL, 3.33 mmol) was added to a stirred solution of diisopropyl azodicarboxylate (1.21 mL, 6.15 mmol), triphenylphosphine (1.346 mL, 6.16 mmol) and methyl 4-bromo-3-hydroxybenzoate (0.9489 g, 4.11 mmol) in tetrahydrofuran (40 mL) and the mixture was stirred at room temperature for 2 days. Water and ethyl acetate was added and the aqueous phase was washed with ethyl acetate. The aqueous phase was acidified (pH˜1) using hydrochloric acid (2 M) and extracted with ethyl acetate. The combined organic phases were washed with water and brine, dried over magnesium sulfate and the solvent was evaporated to give 0.894 g (67% yield) of the title compound. ¹H NMR (500 MHz, CDCl₃) δ ppm 7.64 (d, 1H) 7.53-7.58 (m, 2H) 4.32 (t, 2H) 3.94 (s, 3H) 2.74 (dt, 2H); GC MS (EI) m/z 326, 328 [M]^{+•}.

### EXAMPLE 36

### 4-(3,3-Dicyclopropylprop-1-ynyl)-N-(2-sulfamoylphenylsulfonyl)-3-(3,3,3-trifluoropropoxy)benzamide

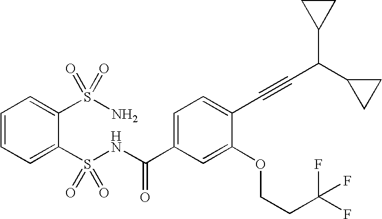

1-(3-Dimethylaminopropyl)-3-ethylcarbodiimide hydrochloride (0.0326 g, 0.17 mmol) was added to a stirred solution of benzene-1,2-disulfonamide (0.0310 g, 0.13 mmol), 4-(3,3-dicyclopropylprop-1-ynyl)-3-(3,3,3-trifluoropropoxy)benzoic acid (0.035 g, 0.10 mmol) and 4-dimethylaminopyridine (0.0181 g, 0.15 mmol) in anhydrous N,N-dimethylformamide (3 mL) and the resulting mixture was stirred at room temperature over night. Water and ethyl acetate was added and the phases were separated. The aqueous phase was acidified (pH˜1) with hydrochloric acid (2 M) and extracted with ethyl acetate. The combined organic phases were washed with water, water/brine (1:1) and brine, dried over magnesium sulfate and the solvent was evaporated. Purification by preparative HPLC is gave 0.024 g (43% yield) of the title compound. ¹H NMR (500 MHz, DMSO-d₆) δ ppm 8.25-8.37 (m, 1H) 8.07-8.17 (m, 1H) 7.76-7.93 (m, 2H) 7.55 (s, 1H) 7.39-7.47 (m, 3H) 7.33-7.38 (m, 1H) 4.23-4.38 (m, 2H) 2.71-2.91 (m, 2H) 2.29-2.34 (m, 1H) 0.96-1.09 (m, 2H) 0.41-0.49 (m, 4H) 0.37 (d, 4H); MS (APCI) m/z 569.2 [M−1]⁻.

### a) 4-(3,3-Dicyclopropylprop-1-ynyl)-3-(3,3,3-trifluoropropoxy)benzoic acid

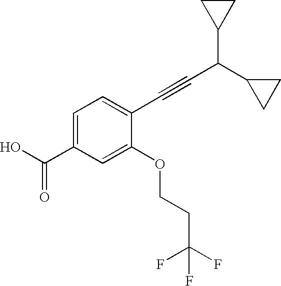

A solution of lithium hydroxide monohydrate (0.1224 g, 2.92 mmol) in water (1.5 mL) was added to a solution of methyl 4-(3,3-dicyclopropylprop-1-ynyl)-3-(3,3,3-trifluoropropoxy)benzoate (0.240 g, 0.66 mmol) in tetrahydrofuran (3 mL) and the resulting mixture was stirred at room temperature over night. Water and ethyl acetate was added. The aqueous phase was acidified (pH˜1) with hydrochloric acid (2 M), and extracted with ethyl acetate. The combined organic phases were dried over magnesium sulfate and the solvent was evaporated. Purification by preparative HPLC gave 0.039 g (17% yield) of the title compound. ¹H NMR (500 MHz, DMSO-d₆) δ ppm 13.14 (br. s., 1H) 7.53-7.58 (m, 1H) 7.50-7.53 (m, 1H) 7.38-7.44 (m, 1H) 4.31 (t, 2H) 2.69-2.89 (m, 2H) 2.32 (s, 1H) 0.99-1.09 (m, 2H) 0.45 (dd, 4H) 0.38 (d, 4H); MS (ESI) m/z 351.2 [M−1]⁻.

### b) Methyl 4-(3,3-dicyclopropylprop-1-ynyl)-3-(3,3,3-trifluoropropoxy)benzoate

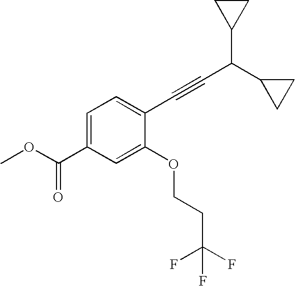

Trifluoroacetic acid (0.26 mL, 3.39 mmol) was added to a stirred solution of methyl 4-(3,3-dicyclopropyl-3-hydroxyprop-1-ynyl)-3-(3,3,3-trifluoropropoxy)benzoate (0.320 g, 0.84 mmol) and triethylsilane (0.27 mL, 1.69 mmol) in dichloromethane (4 mL) and the mixture was stirred at room temperature for 10 min. Sodium carbonate (0.4827 g, 4.55 mmol) was added and the mixture was stirred for 5 min. Water and dichloromethane was added and the aqueous phase was extracted with dichloromethane. The combined organic phases were washed with water and brine, dried over magnesium sulfate and the solvent was evaporated to give 27% yield of the title compound. GC MS (EI) m/z 366 [M]^{+•}.

### c) Methyl 4-(3,3-dicyclopropyl-3-hydroxyprop-1-ynyl)-3-(3,3,3-trifluoropropoxy)benzoate

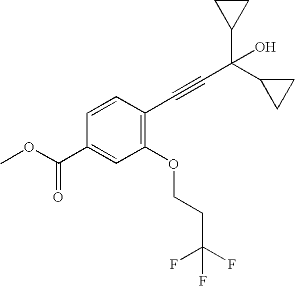

Copper(I) iodide (0.012 mL, 0.32 mmol) was added to a stirred solution of methyl 4-bromo-3-(3,3,3-trifluoropropoxy)benzoate (0.6628 g, 2.03 mmol), 1,1-dicyclopropylprop-2-yn-1-ol (0.590 g, 4.33 mmol), tetrakis(triphenylphosphine)palladium(0) (0.2154 g, 0.19 mmol) and triethylamine (2.0 mL, 14.35 mmol) in anhydrous N,N-dimethylformamide (13 mL) under an atmosphere of nitrogen. The resulting mixture was heated at 60° C. over the weekend. Water and ethyl acetate was added and the aqueous phase was extracted with ethyl acetate. The combined organic phases were washed with water, water/brine (1:1) and brine, dried over magnesium sulfate and the solvent was evaporated. Purification by column chromatography, using a gradient of ethyl acetate in n-heptane (0-20%) as the eluent, gave 0.777 g (100% yield) of the title compound. ¹H NMR (500 MHz, CDCl₃) δ ppm 7.61 (dd, 1H) 7.49 (s, 1H) 7.41 (d, 1H) 4.29 (t, 2H) 3.93 (s, 3H) 2.73 (dt, 2H) 2.14 (s, 1H) 1.30-1.39 (m, 2H) 0.72 (dt, 2H) 0.45-0.65 (m, 8H); GC MS (EI) m/z 382 [M]⁺.

### EXAMPLE 37

### 3-Methoxy-4-(3-methylhex-1-ynyl)-N-(2-sulfamoylphenylsulfonyl)benzamide

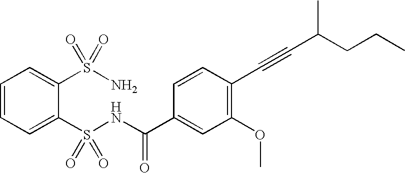

The title compound was synthesized as described for Example 31 in 30% yield, starting from 4-bromo-3-methoxy-N-(2-sulfamoylphenylsulfonyl)benzamide and 3-methylhex-1-yne.

After purification by preparative HPLC dichloromethane was added followed by n-heptane until a milky solution was obtained. The solid was removed by filtration and dried in vacuo. ¹H NMR (500 MHz, DMSO-d₆) δ ppm 8.27-8.37 (m, 1H) 8.10-8.17 (m, 1H) 7.79-7.93 (m, 2H) 7.48-7.53 (m, 1H) 7.44 (s, 2H) 7.39-7.42 (m, 1H) 7.32-7.38 (m, 1H) 3.83 (s, 3H) 2.66-2.78 (m, 1H) 1.37-1.56 (m, 4H) 1.19 (d, 3H) 0.91 (s, 3H); MS (APCI) m/z 463.1 [M−1]⁻.

### EXAMPLE 38

### 3-Methoxy-N-(2-sulfamoylphenylsulfonyl)-4-((4-(trifluoromethyl)phenyl)ethynyl)benzamide

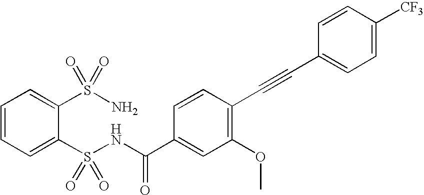

The title compound was synthesized as described for Example 31 in 44% yield, starting from 4-bromo-3-methoxy-N-(2-sulfamoylphenylsulfonyl)benzamide and 1-ethynyl-4-(trifluoromethyl)benzene. After purification by preparative HPLC dichloromethane was added followed by n-heptane the formed solid was removed by filtration and dried in vacuo. ¹H NMR (500 MHz, DMSO-d₆) δ ppm 8.29-8.40 (m, 1H) 8.10-8.19 (m, 1H) 7.84-7.94 (m, 2H) 7.78-7.83 (m, 2H) 7.73-7.78 (m, 2H) 7.58-7.65 (m, 2H) 7.49-7.54 (m, 1H) 7.46 (br. s., 2H) 3.92 (s, 3H); MS (APCI) m/z 537.1 [M−1]⁻.

### EXAMPLE 39

### 3-(Hydroxymethyl)-N-(2-sulfamoylphenylsulfonyl)-4-((4-(trifluoromethyl)phenyl)ethynyl)benzamide

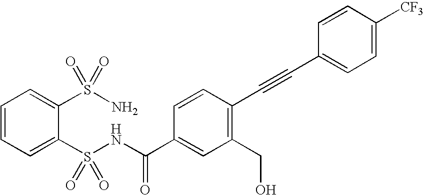

The title compound was synthesized as described for Example 31 in 9% yield, starting from 4-bromo-3-(hydroxymethyl)-N-(2-sulfamoylphenylsulfonyl)benzamide and 1-ethynyl-4-(trifluoromethyl)benzene. Purification by preparative HPLC. The fractions containing product were pooled and the acetonitrile was evaporated. The aqueous phase was washed with dichloromethane, acidified with hydrochloric acid (2 M) and extracted with ethyl acetate. The combined organic phases were washed with water and brine, dried over magnesium sulfate and the solvent was evaporated. ¹H NMR (500 MHz, DMSO-d₆) δ ppm 8.23-8.34 (m, 1H) 8.10-8.14 (m, 1H) 8.05-8.10 (m, 1H) 7.78-7.87 (m, 7H) 7.59-7.65 (m, 2H) 7.44 (br. s., 2H) 4.74 (s, 2H); MS (APCI) m/z 537.1 [M−1]⁻.

### EXAMPLE 40

### 4-(3-Methylhex-1-ynyl)-N-(2-sulfamoylphenylsulfonyl)benzamide

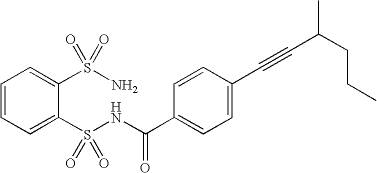

The title compound was synthesized as described for Example 31 in 41% yield, starting from 4-iodo-N-(2-sulfamoylphenylsulfonyl)benzamide and 3-methylhex-1-yne. After purification by preparative HPLC dichloromethane was added and the product trituated with heptane, filtered and dried in vacuo. ¹H NMR (500 MHz, DMSO-d₆) δ ppm 8.29-8.38 (m, 1H) 8.09-8.19 (m, 1H) 7.86-7.94 (m, 2H) 7.82-7.86 (m, 2H) 7.39-7.49 (m, 4H) 2.66-2.79 (m, 1H) 1.36-1.56 (m, 4H) 1.20 (d, 3H) 0.91 (t, 3H); MS (ESI) m/z 433.2 [M−1]⁻.

### EXAMPLE 41

### N-(2-Sulfamoylphenylsulfonyl)-4-((4-(trifluoromethyl)phenyl)ethynyl)benzamide

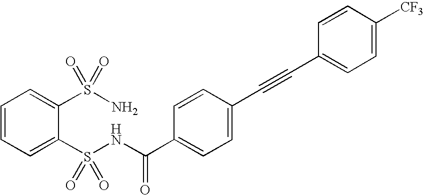

The title compound was synthesized as described for Example 31 in 43% yield, starting from 1-ethynyl-4-(trifluoromethyl)benzene and 4-iodo-N-(2-sulfamoylphenylsulfonyl)benzamide The product was washed with dichloromethane and dried in vacuo. ¹H NMR (500 MHz, DMSO-d₆) δ ppm 8.26-8.35 (m, 1H) 8.08-8.15 (m, 1H) 7.92-7.97 (m, 2H) 7.78-7.86 (m, 6H) 7.64-7.70 (m, 2H) 7.45 (br. s., 2H); MS (APCI) m/z 507 [M−1]⁻.

### EXAMPLE 42

### 4-(6-Chlorohex-1-ynyl)-N-(2-sulfamoylphenylsulfonyl)benzamide

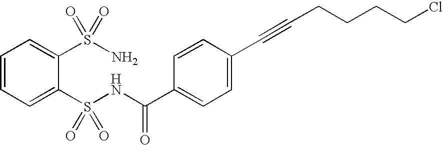

The title compound was synthesized as described for Example 31 in 53% yield, starting from 6-chloro-1-hexyne and 4-iodo-N-(2-sulfamoylphenylsulfonyl)benzamide. ¹H NMR (500 MHz, CDCl₃) δ ppm 9.44 (br. s., 1H) 8.49-8.62 (m, 1H) 8.24-8.34 (m, 1H) 7.78-7.90 (m, 2H) 7.69-7.77 (m, 2H) 7.41-7.47 (m, 2H) 5.68-5.72 (m, 2H) 3.62 (t, 2H) 2.49 (t, 2H) 1.89-1.99 (m, 2H) 1.73-1.83 (m, 2H); MS (ESI) m/z 453.2 [M−1]⁻.

### EXAMPLE 43

### N-(2-Sulfamoylphenylsulfonyl)-6-β4-(trifluoromethyl)phenyl)ethynyl)nicotinamide

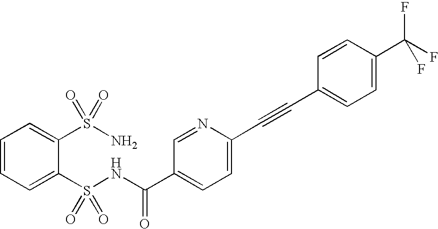

Triethylamine (1.67 mL, 12.0 mmol) was added to a mixture of 6-bromo-N-(2-sulfamoylphenylsulfonyl)nicotinamide (0.17 g, 0.40 mmol), 4-ethynyl-alpha,alpha,alpha-trifluorotoluene (0.19 mL, 1.20 mmol) and tetrakis(triphenylphosphine)palladium(0) (0.046 g, 0.040 mmol) in N,N-dimethylformamide (2 mL) was and the mixture was stirred for 5 minutes. Copper(I) iodide (0.011 g, 0.060 mmol) was added and the reaction was heated at 65° C. over night. The reaction mixture was partitioned between ethyl acetate and diluted hydrochloric acid, the organic phase was dried over magnesium sulfate and the solvent was evaporated. Purification by preparative HPLC, fractions containing the product was pooled, diluted hydrochloric acid was added and the mixture was extracted with dichloromethane. The organic phase was dried over magnesium sulfate and evaporated to give 0.109 g (53% yield) of the title compound. ¹H NMR (DMSO-d₆) δ ppm 9.03 (d, 1H) 8.21-8.35 (m, 2H) 8.07-8.15 (m, 1H) 7.74-7.91 (m, 7H) 7.47 (br. s., 2H); MS (ESI) m/z 508 [M−1]⁻.

### a) 6-Bromo-N-(2-sulfamoylphenylsulfonyl)nicotinamide

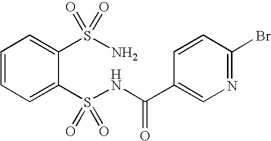

1-(3-Dimethylaminopropyl)-3-ethylcarbodiimide hydrochloride (0.508 g, 2.65 mmol) was added to a solution of 6-bromonicotinic acid (0.357 g, 1.77 mmol), benzene-1,2-disulfonamide (0.418 g, 1.77 mmol) and 4-dimethylaminopyridine (0.318 g, 2.60 mmol) in N,N-dimethylformamide (20 mL) at room temperature and the mixture was stirred over night. Water was added and the aqueous phase was washed with ethyl acetate. The aqueous phase was acidified (pH˜2) with 2 M hydrochloric acid and extracted with ethyl acetate. The organic phase was washed with water and water/brine (1:1), dried over magnesium sulfate and the solvent was evaporated to give 0.677 g (91% yield) of the title compound. ¹H NMR (400 MHz, DMSO-d₆) δ ppm 8.80 (d, 1H) 8.29-8.37 (m, 1H) 8.08-8.16 (m, 2 H) 7.81-7.92 (m, 2H) 7.78 (d, 1H) 7.46 (m, 1H); MS (ESI) m/z 420.0 [M+H]⁺, 421.8 [M−H].

### EXAMPLE 44

### 5-Chloro-6-(3-methylbut-1-ynyl)-N-(2-sulfamoylphenylsulfonyl)nicotinamide

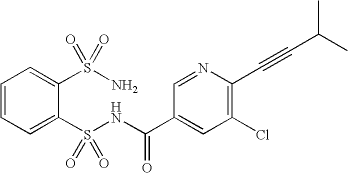

A mixture of 5,6-dichloro-N-(2-sulfamoylphenylsulfonyl)nicotinamide (0.16 g, 0.40 mmol), 3-methyl-1-butyne (0.12 mL, 1.20 mmol), copper(I) iodide (7.6 mg, 0.040 mmol), bis(triphenylphosphine)palladium(II) chloride (0.028 g, 0.040 mmol) and diisopropylamine (0.17 mL, 1.20 mmol) in N,N-dimethylformamide (3 mL) under an atmosphere of argon was heated in a microwave at 100° C. for 2 h. The reaction mixture was partitioned between ethyl acetate and diluted hydrochloric acid, the organic phase was dried over magnesium sulfate and evaporated. The residue was purified by preparative HPLC, fractions containing the product was pooled, diluted hydrochloric acid was added and the mixture was extracted with dichloromethane. The organic phase was dried over magnesium sulfate and evaporated to give 0.063 g (36% yield) of the title compound. ¹H NMR (DMSO-d₆) δ ppm 8.82 (d, 1H) 8.24-8.32 (m, 2H) 8.06-8.11 (m, 1H) 7.74-7.82 (m, 2H) 7.46 (br. s., 2H) 2.87-2.98 (m, 1H) 1.25 (d, 6H); MS (ESI) m/z 440 [M−1]⁻.

### a) 5,6-Dichloro-N-(2-sulfamoylphenylsulfonyl)nicotinamide

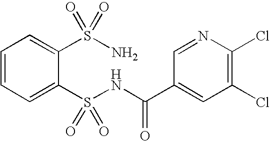

The title compound was synthesized as described for Example 43a) in 88% yield, starting from 5,6-dichloronicotinic acid. ¹H NMR (DMSO-d₆) δ ppm 8.71-8.77 (m, 1H) 8.36-8.43 (m, 1H) 8.23-8.31 (m, 1H) 8.05-8.11 (m, 1H) 7.72-7.81 (m, 2H) 7.43-7.50 (m, 2H); MS (ESI) m/z 408 [M−1]⁻.

### EXAMPLE 45

### 4-(3-Methylbut-1-ynyl)-N-(2-sulfamoylphenylsulfonyl)-3-(3,3,3-trifluoropropoxy)benzamide

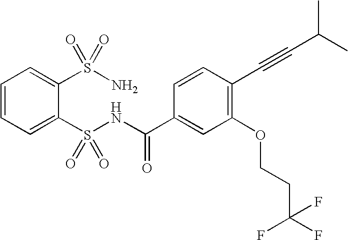

The title compound was synthesized as described for Example 44 in 16% yield, starting from 4-bromo-N-(2-sulfamoylphenylsulfonyl)-3-(3,3,3-trifluoropropoxy)benzamide. Purification by preparative HPLC. ¹H NMR (DMSO-d₆) δ ppm 8.27 (d, 1H) 8.07 (d, 1H) 7.73-7.88 (m, 2H) 7.49 (s, 1H) 7.27-7.42 (m, 4H) 4.21 (t, 2H) 2.68-2.84 (m, 3H) 1.12 (d, 6H); MS (ESI) m/z 517 [M−1]⁻.

### EXAMPLE 46

### 5-Fluoro-6-(3-methylbut-1-ynyl)-N-(2-sulfamoylphenylsulfonyl)nicotinamide

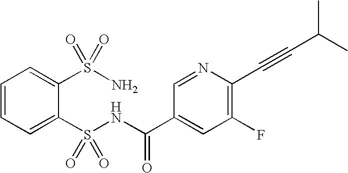

The title compound was synthesized as described for Example 44 in 10% yield, starting from 6-chloro-5-fluoro-N-(2-sulfamoylphenylsulfonyl)nicotinamide. Purification by preparative HPLC. ¹H NMR (DMSO-d₆) δ ppm 8.68-8.74 (m, 1H) 8.18-8.24 (m, 1H) 7.92-8.05 (m, 2H) 7.65-7.76 (m, 2H) 7.39 (br. s., 2H) 2.80-2.92 (m, 1H) 1.18 (d, 6H); MS (ESI) m/z 424 [M−1]⁻.

### a) 6-Chloro-5-fluoro-N-(2-sulfamoylphenylsulfonyl)nicotinamide

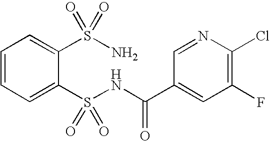

N-(3-Dimethylaminopropyl)-N′-ethylcarbodiimide hydrochloride (0.99 g, 5.15 mmol) and 4-(dimethylamino)pyridine (0.63 g, 5.15 mmol) were added to a solution of benzene-1,2-disulfonamide (0.81 g, 3.43 mmol) and 6-chloro-5-fluoronicotinic acid (0.60 g, 3.43 mmol) in N,N-dimethylformamide (25 mL), the resulting mixture was stirred at room temperature over night. Water was added and the mixture was extracted with ethyl acetate, the aqueous phase was acidified with diluted hydrochloric acid and extracted with ethyl acetate. The organic phase was dried over magnesium sulfate and evaporated to give 0.33 g (24% yield) of the title compound. MS (ESI) m/z 392 [M−1]⁻.

### b) 6-Chloro-5-fluoronicotinic acid

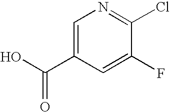

Potassium permanganate (1.09 g, 6.87 mmol) was added to a mixture of 2-chloro-3-fluoro-5-methylpyridine (0.5 g, 3.43 mmol), water (2 mL) and pyridine (2 mL) and the mixture was heated at 100° C. for 1 h. Solid material stuck in the reflux condenser was washed down with pyridine. More potassium permanganate (2.17 g, 13.7 mmol) was added in portions and heating at 100° C. was continued for a total of 3 h. The reaction mixture was filtered to remove the solids and the filtrate was concentrated to give the crude title compound. MS (ESI) m/z 174 [M−1]⁻.

### EXAMPLE 47

### 5-Fluoro-N-(2-sulfamoylphenylsulfonyl)-6-((4-(trifluoromethyl)phenyl)ethynyl)nicotinamide

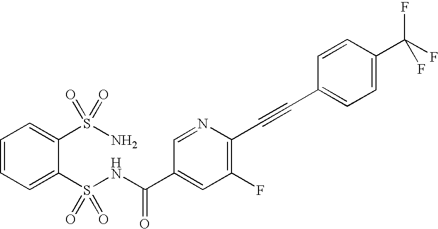

The title compound was synthesized as described for Example 44 in 20% yield, starting from 6-chloro-5-fluoro-N-(2-sulfamoylphenylsulfonyl)nicotinamide and 4-ethynyl-alpha,alpha,alpha-trifluorotoluene. Purification by preparative HPLC. ¹H NMR (DMSO-d₆) δ ppm 8.89 (t, 1H) 8.25 (dd, 1H) 8.03-8.12 (m, 2H) 7.84-7.92 (m, 4H) 7.68-7.77 (m, 2H) 7.45 (s, 2H); MS (ESI) m/z 526 [M−1]⁻.

### EXAMPLE 48

### N-(2-Sulfamoylphenylsulfonyl)-6-β4-(trifluoromethyl)phenyl)ethynyl)-5-(3,3,3-trifluoropropoxy)nicotinamide

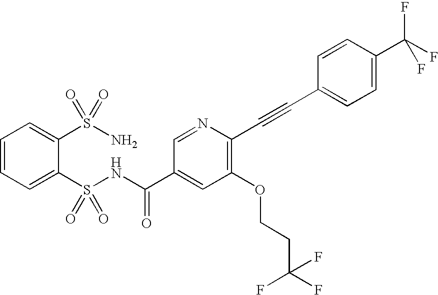

The title compound was synthesized as described for Example 44 in 39% yield, starting from 6-chloro-N-(2-sulfamoylphenylsulfonyl)-5-(3,3,3-trifluoropropoxy)nicotinamide and 4-ethynyl-alpha,alpha,alpha-trifluorotoluene and heating the reaction for 3 h. Purification by preparative HPLC. ¹H NMR (DMSO-d₆) δ ppm 8.56 (d, 1H) 8.24-8.31 (m, 1H) 8.03-8.10 (m, 1H) 7.93 (s, 1H) 7.75-7.83 (m, 4H) 7.71 (d, 2H) 7.42 (s, 2H) 4.34 (t, 2H) 2.81-2.94 (m, 2H); MS (ESI) m/z 620 [M−1]⁻.

### a) 6-Chloro-N-(2-sulfamoylphenylsulfonyl)-5-(3,3,3-trifluoropropoxy)nicotinamide

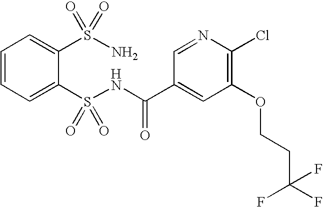

The title compound was synthesized as described for Example 46a) in 61% yield, starting from 6-chloro-5-(3,3,3-trifluoropropoxy)nicotinic acid. ¹H NMR (DMSO-d₆) δ ppm 8.43 (d, 1H) 8.36 (d, 1H) 8.13-8.18 (m, 1H) 8.01 (d, 1H) 7.85-7.93 (m, 2H) 7.50 (s, 2H) 4.41 (t, 2H), 2.9 (m, 2H); MS (ESI) m/z 486 [M−1]⁻.

### b) 6-Chloro-5-(3,3,3-trifluoropropoxy)nicotinic acid

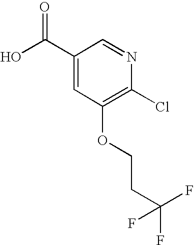

A solution of lithium hydroxide (0.12 g, 4.85 mmol) in water (2 mL) was added to a solution of methyl 6-chloro-5-(3,3,3-trifluoropropoxy)nicotinate (0.46 g, 1.62 mmol) in tetrahydrofuran (6 mL) and methanol (2 mL), the resulting mixture was stirred at room temperature over night. Water and hydrochloric acid (2 M) was added and the mixture was extracted with ethyl acetate. The organic phase was dried over magnesium sulfate and evaporated to give 0.42 g (96% yield) of the title compound. ¹H NMR (DMSO-d₆) δ ppm 13.70 (br. s., 1H) 8.51 (d, 1H) 7.95 (d, 1H) 4.46 (t, 2H) 2.80-2.94 (m, 2H); MS (ESI) m/z 268 [M−1]⁻.

### c) Methyl 6-chloro-5-(3,3,3-trifluoropropoxy)nicotinate

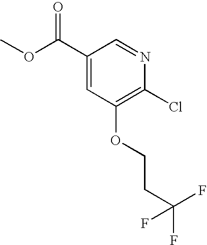

3,3,3-Trifluoro-1-propanol (0.39 mL, 4.50 mmol), triphenylphosphine (1.18 g, 4.50 mmol) and diisopropyl azodicarboxylate (0.89 mL, 4.50 mmol) were added to a solution of methyl 6-chloro-5-hydroxynicotinate (0.56 g, 3.0 mmol) in tetrahydrofuran (20 mL). The reaction was stirred over night, concentrated and purified by column chromatography, using heptane/ethyl acetate (5:1) as the eluent, to give 0.46 g (54% yield) of the title compound. ¹H NMR (DMSO-d₆) δ ppm 8.52 (d, 1H) 7.97 (d, 1H) 4.46 (t, 2H) 3.90 (s, 3H) 2.78-2.95 (m, 2H); MS (ESI) m/z 284 [M+1]⁺.

### EXAMPLE 49

### 6-(3-Methylbut-1-ynyl)-N-(2-sulfamoylphenylsulfonyl)-5-(3,3,3-trifluoroprop oxy)nicotinamide

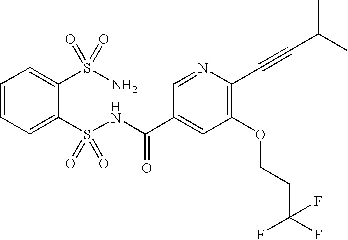

The title compound was synthesized as described for Example 44 in 23% yield, starting from 6-chloro-N-(2-sulfamoylphenylsulfonyl)-5-(3,3,3-trifluoropropoxy)nicotinamide and heating the reaction for 3 h. Purification by preparative HPLC. ¹H NMR (DMSO-d₆) δ ppm 8.45 (d, 1H) 8.25 (br. s., 1H) 8.02-8.09 (m, 1H) 7.73-7.85 (m, 3H) 7.40 (s, 2H) 4.24 (t, 2H) 2.71-2.86 (m, 3H) 1.14 (d, 6H); MS (ESI) m/z 518 [M−1]⁻.

### EXAMPLE 50

### 3-Methyl-4-(3-methylbut-1-ynyl)-N-(2-sulfamoylphenylsulfonyl)benzamide

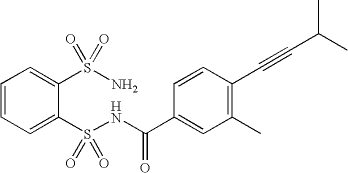

The title compound was synthesized as described for Example 44 in 25% yield, starting from 4-bromo-3-methyl-N-(2-sulfamoylphenylsulfonyl)benzamide and heating the reaction for 3 h. Purification by preparative HPLC. ¹H NMR (DMSO-d₆) δ ppm 8.32 (d, 1H) 8.14 (d, 1H) 7.82-7.94 (m, 2H) 7.80 (s, 1H) 7.66 (dd, 1H) 7.35-7.48 (m, 3H) 2.81-2.94 (m, 1H) 2.37 (s, 3H) 1.24 (d, 6H); MS (ESI) m/z 419 [M−1]⁻.

### a) 4-Bromo-3-methyl-N-(2-sulfamoylphenylsulfonyl)benzamide

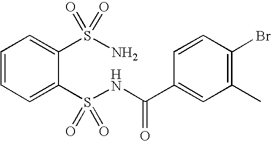

The title compound was synthesized as described for Example 8a) in 59% yield, starting from 4-bromo-3-methylbenzoic acid. ¹H NMR (400 MHz, DMSO-d₆) δ ppm 8.18 (d, 1H), 8.03 (d, 1H), 7.84 (s, 1H), 7.62-7.74 (m, 2H), 7.51-7.62 (m, 2H), 7.42 (s, 2H), 2.35 (s, 3H); MS (ESI) m/z 433, 435 [M+1]⁺; MS (ESI) m/z 431, 433 [M−1]⁻.

### EXAMPLE 51

### 4-(3-Methylbut-1-ynyl)-N-(2-sulfamoylphenylsulfonyl)-3-(2-(2,2,2-trifluoroethoxy)ethoxy)benzamide

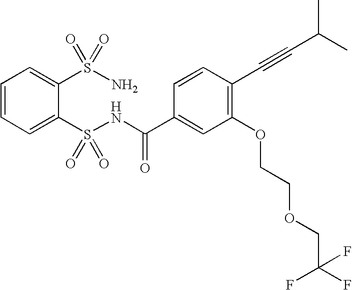

The title compound was synthesized as described for Example 43 in 36% yield, starting from 4-bromo-N-(2-sulfamoylphenylsulfonyl)-3-(2-(2,2,2-trifluoroethoxy)ethoxy)benzamide and 3-methylbut-1-yne. Purification by preparative HPLC. ¹H NMR (DMSO-d₆) δ ppm 8.27 (d, 1H) 8.07 (dd, 1H) 7.75-7.88 (m, 2H) 7.46 (d, 1H) 7.29-7.41 (m, 4H) 4.12-4.22 (m, 4H) 3.90-3.97 (m, 2H) 2.68-2.80 (m, 1H) 1.13 (d, 6H); MS (ESI) m/z 547 [M−1]⁻.

### EXAMPLE 52

### 3-(2,2-Difluoroethoxy)-4-(3-methylbut-1-ynyl)-N-(2-sulfamoylphenylsulfonyl)benzamide

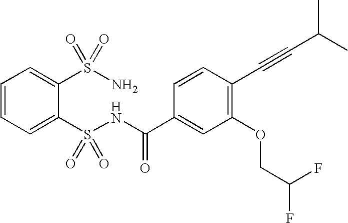

The title compound was synthesized as described for Example 43 in 49% yield, starting from 4-bromo-3-(2,2-difluoroethoxy)-N-(2-sulfamoylphenylsulfonyl)benzamide and 3-methylbut-1-yne. Purification by preparative HPLC. ¹H NMR (DMSO-d₆) δ ppm 8.34 (d, 1H) 8.14 (d, 1H) 7.81-7.95 (m, 2H) 7.59 (s, 1H) 7.37-7.52 (m, 4H) 6.26-6.56 (m, 1H) 4.40 (td, 2H) 2.77-2.90 (m, 1H) 1.21 (d, 6H); MS (APCI) m/z 485 [M−1]⁻.

### a) 4-Bromo-3-(2,2-difluoroethoxy)-N-(2-sulfamoylphenylsulfonyl)benzamide

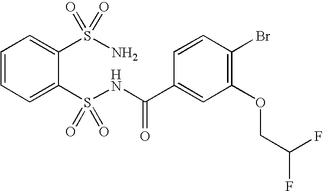

The title compound was synthesized as described for Example 46a) in 62% yield, starting from 4-bromo-3-(2,2-difluoroethoxy)benzoic acid. ¹H NMR (DMSO-d₆) δ ppm 8.29 (dd, 1H) 8.09 (dd, 1H) 7.79-7.87 (m, 2H) 7.67 (d, 1H) 7.60 (d, 1H) 7.34-7.44 (m, 3H) 6.24-6.54 (m, 1H) 4.41 (td, 2H); MS (ESI) m/z 497, 499 [M−1]⁻.

### b) 4-Bromo-3-(2,2-difluoroethoxy)benzoic acid

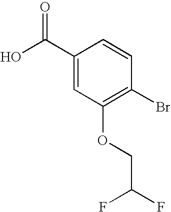

The title compound was synthesized as described for Example 48b) in 95% yield, starting from methyl 4-bromo-3-(2,2-difluoroethoxy)benzoate. ¹H NMR (DMSO-d₆) δ ppm 13.29 (br. s., 1H) 7.76 (d, 1H) 7.62 (d, 1H) 7.52 (dd, 1H) 6.29-6.60 (m, 1H) 4.52 (td, 2H); MS (ESI) m/z 279, 281 [M−1]⁻.

### c) Methyl 4-bromo-3-(2,2-difluoroethoxy)benzoate

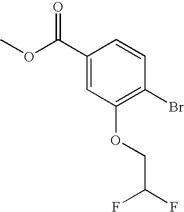

The title compound was synthesized as described for Example 48c) in 94% yield, starting from 2,2-difluoroethanol. Purification by column chromatography, using a gradient of ethyl acetate (0-30%) in heptane as the eluent. ¹H NMR (DMSO-d₆) δ ppm 7.80 (d, 1H) 7.64 (d, 1H) 7.54 (dd, 1H) 6.30-6.59 (m, 1H) 4.54 (td, 2H) 3.88 (s, 3H); GC MS (EI) m/z 294, 296 [M]^{+•}.

### EXAMPLE 53

### 4-(3-Methylbut-1-ynyl)-N-(2-sulfamoylphenylsulfonyl)-3-(2,2,2-trifluoroethoxy)benzamide

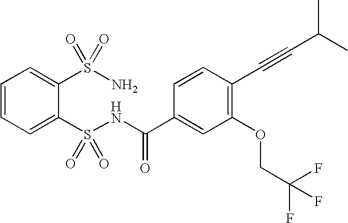

The title compound was synthesized as described for Example 43 in 37% yield, starting from 4-bromo-N-(2-sulfamoylphenylsulfonyl)-3-(2,2,2-trifluoroethoxy)benzamide and 3-methylbut-1-yne. Purification by preparative HPLC. ¹H NMR (DMSO-d₆) δ ppm 8.32 (d, 1H) 8.13 (d, 1H) 7.80-7.92 (m, 2H) 7.61 (s, 1H) 7.54 (dd, 1H) 7.39-7.49 (m, 3H) 4.84 (q, 2H) 2.77-2.90 (m, 1H) 1.21 (d, 6H); MS (APCI) m/z 503 [M−1]⁻.

### a) 4-Bromo-N-(2-sulfamoylphenylsulfonyl)-3-(2,2,2-trifluoroethoxy)benzamide

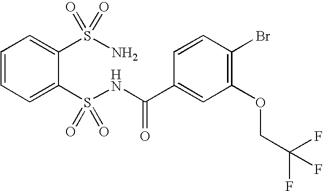

The title compound was synthesized as described for Example 46a) in 24% yield, starting from 4-bromo-3-(2,2,2-trifluoroethoxy)benzoic acid. ¹H NMR (DMSO-d₆) δ ppm 8.31-8.40 (m, 1H) 8.11-8.19 (m, 1H) 7.84-7.94 (m, 2H) 7.76 (d, 1H) 7.70 (d, 1H) 7.41-7.54 (m, 3H) 4.92 (q, 2H); MS (ESI) m/z 515, 517 [M−1]⁻.

### b) 4-Bromo-3-(2,2,2-trifluoroethoxy)benzoic acid

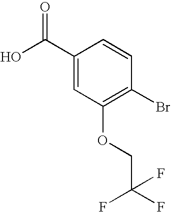

The title compound was synthesized as described for Example 48b) in 99% yield, starting from methyl 4-bromo-3-(2,2,2-trifluoroethoxy)benzoate. ¹H NMR (DMSO-d₆) δ ppm 13.26 (br. s., 1H) 7.80 (d, 1H) 7.71 (d, 1H) 7.62 (d, 1H) 4.92 (q, 2H); MS (ESI) m/z 297, 299 [M−1]⁻.

### c) Methyl 4-bromo-3-(2,2,2-trifluoroethoxy)benzoate

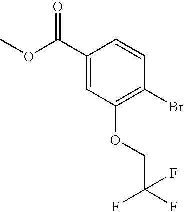

2,2,2-Trifluoroethyl p-toluenesulfonate (0.76 g, 3.00 mmol) was added to a slurry of methyl 4-bromo-3-hydroxybenzoate (0.46 g, 2.00 mmol) and potassium carbonate (0.83 g, 6.00 mmol) in N,N-dimethylformamide (5 mL). The reaction was heated at 100° C. over night, partitioned between water and dichloromethane. The organic phase was dried over magnesium sulfate and evaporated. Purification by column chromatography, using a gradient of ethyl acetate (0-30%) in heptane as the eluent, gave the title compound, containing some impurities. ¹H NMR (DMSO-d₆) δ ppm 7.87 (d, 1H) 7.81 (d, 1H) 7.70 (d, 1H) 5.01 (q, 2H) 3.87 (s, 3H).

### EXAMPLE 54

### 4-(3-Methylbut-1-ynyl)-N-(2-sulfamoylphenylsulfonyl)-3-(trifluoromethoxy)benzamide

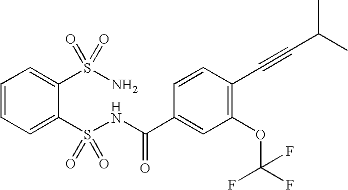

The title compound was synthesized as described for Example 46a) in 13% yield, starting from 4-(3-methylbut-1-ynyl)-3-(trifluoromethoxy)benzoic acid. ¹H NMR (DMSO-d₆) δ ppm 8.36-8.42 (m, 1H) 8.16-8.23 (m, 1H) 7.85-8.02 (m, 4H) 7.69 (d, 1H) 7.52 (br. s., 2H) 2.88-3.02 (m, 1H) 1.29 (d, 6H); MS (APCI) m/z 489 [M−1]⁻.

### a) 4-(3-Methylbut-1-ynyl)-3-(trifluoromethoxy)benzoic acid

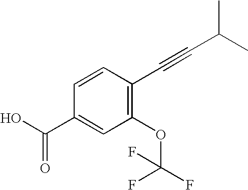

n-Butyllithium (2.5 M in hexane, 0.54 mL, 1.34 mmol) was added dropwise to a cooled (−78° C.) solution of 4-bromo-1-(3-methylbut-1-ynyl)-2-(trifluoromethoxy)benzene (0.34 g, 1.12 mmol) in tetrahydrofuran (5 mL), the reaction mixture was stirred at −78° C. for 1 h and then poured onto freshly crushed dry-ice. After attaining room temperature water and hydrochloric acid (2 M) was added and the mixture was extracted with ethyl acetate. The organic phase was dried over magnesium sulfate and evaporated to give 0.19 g (63% yield) of the title compound. ¹H NMR (DMSO-d₆) δ ppm 7.86 (dd, 1H) 7.77 (s, 1H) 7.62 (d, 1H) 2.79-2.89 (m, 1H) 1.17 (d, 6H); MS (ESI) m/z 271 [M−1]⁻.

### b) 4-Bromo-1-(3-methylbut-1-ynyl)-2-(trifluoromethoxy)benzene

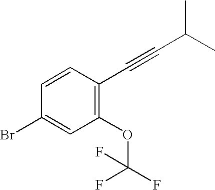

The title compound was synthesized as described for Example 43 in 82% yield, starting from 4-bromo-1-iodo-2-(trifluoromethoxy)benzene and 3-methylbut-1-yne. Purification by column chromatography, using a gradient of ethyl acetate (0-30%) in heptane as the eluent. ¹H NMR (DMSO-d₆) δ ppm 7.67 (s, 1H) 7.56 (dd, 1H) 7.43 (d, 1H) 2.72-2.83 (m, 1H) 1.14 (d, 6H); GC MS (EI) m/z 306, 308 [M]^{+•}.

### EXAMPLE 55

### 4-(3-Methylbut-1-ynyl)-N-(2-sulfamoylphenylsulfonyl)-3-((3,3,3-trifluoropropoxy)methyl)benzamide

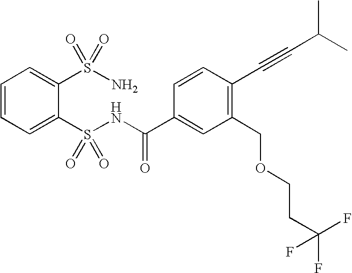

The title compound was synthesized as described for Example 43 starting from 4-bromo-N-(2-sulfamoylphenylsulfonyl)-3-((3,3,3-trifluoropropoxy)methyl)benzamide and 3-methylbut-1-yne. Purification by preparative HPLC. ¹H NMR (DMSO-d₆) δ ppm 8.34 (dd, 1H) 8.11-8.18 (m, 1H) 7.84-7.96 (m, 3H) 7.81 (dd, 1H) 7.36-7.51 (m, 3H) 4.59 (s, 2H) 3.71 (t, 2H) 2.86 (m, 1H) 2.53-2.70 (m, 2H) 1.23 (d, 6H); MS (APCI) m/z 531 [M−1]⁻.

### a) 4-Bromo-N-(2-sulfamoylphenylsulfonyl)-3-((3,3,3-trifluoropropoxy)methyl)benzamide

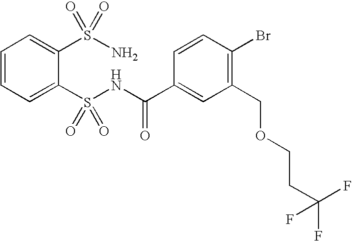

The title compound was synthesized as described for Example 46a) in 29% yield, starting from 4-bromo-3-((3,3,3-trifluoropropoxy)methyl)benzoic acid. MS (ESI) m/z 543, 545 [M−1]⁻.

### b) 4-Bromo-3-((3,3,3-trifluoropropoxy)methyl)benzoic acid

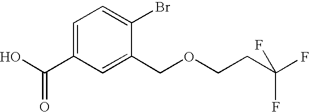

The title compound was synthesized as described for Example 48b) in 85% yield, starting from methyl 4-bromo-3-((3,3,3-trifluoropropoxy)methyl)benzoate. ¹H NMR (DMSO-d₆) δ ppm 7.97 (d, 1H) 7.68-7.76 (m, 2H) 4.52 (s, 2H) 3.71 (t, 2H) 2.53-2.64 (m, 2H); MS (ESI) m/z 327, 325 [M−1]⁻.

### c) Methyl 4-bromo-3-((3,3,3-trifluoropropoxy)methyl)benzoate

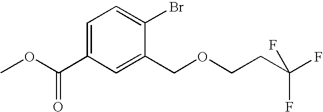

3,3,3-Trifluoro-1-propanol (0.35 mL, 4.00 mmol) was added dropwise to a slurry of sodium hydride (60% in mineral oil, 0.18 g, 4.40 mmol) in tetrahydrofuran (10 mL) and the mixture was stirred for 10 minutes. A solution of methyl 4-bromo-3-(bromomethyl)benzoate (0.62 g, 2.00 mmol) in tetrahydrofuran (5 mL) was added. The reaction was stirred over night and partitioned between saturated ammonium chloride and chloroform. The organic phase was dried over magnesium sulfate and the solvent was evaporated. GC MS (EI) m/z 340, 342 [M]^{+•}.

### EXAMPLE 56

### 4-(3-Methylbut-1-ynyl)-N-(2-sulfamoylphenylsulfonyl)-3-((2,2,2-trifluoroethoxy)methyl)benzamide

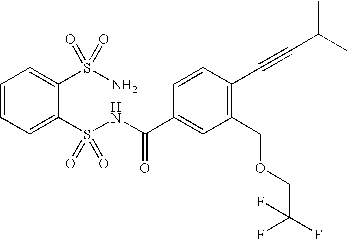

The title compound was synthesized as described for Example 43 in 13% yield, starting from 4-bromo-N-(2-sulfamoylphenylsulfonyl)-3-((2,2,2-trifluoroethoxy)methyl)benzamide and 3-methylbut-1-yne. Purification by preparative HPLC. ¹H NMR (DMSO-d₆) δ ppm 8.11-8.20 (m, 1H) 7.92-8.00 (m, 1H) 7.62-7.78 (m, 4H) 7.20-7.33 (m, 3H) 4.57 (s, 2H) 4.00 (q, 2H) 2.62-2.75 (m, 1H) 1.05 (d, 6H); MS (APCI) m/z 517 [M−1]⁻.

### a) 4-Bromo-N-(2-sulfamoylphenylsulfonyl)-3-((2,2,2-trifluoroethoxy)methyl)benzamide

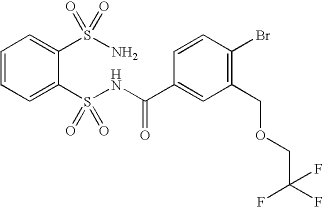

The title compound was synthesized as described for Example 46a) starting from 4-bromo-3-((2,2,2-trifluoroethoxy)methyl)benzoic acid affording the crude title compound. MS (ESI) m/z 529, 531 [M−1]⁻.

### b) 4-Bromo-3-((2,2,2-trifluoroethoxy)methyl)benzoic acid

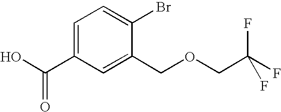

2,2,2-Trifluoroethanol (0.29 mL, 4.00 mmol) was added dropwise to a slurry of sodium hydride (60% in mineral oil, 0.18 g, 4.40 mmol) in tetrahydrofuran (10 mL) and the mixture was stirred for 10 minutes. A solution of methyl 4-bromo-3-(bromomethyl)benzoate (0.62 g, 2.00 mmol) in tetrahydrofuran (5 mL) was added. The reaction was stirred for 3 days, methanol (5 mL) and a solution of lithium hydroxide (0.14 g, 6.00 mmol) in water (3 mL) was added and the resulting mixture was stirred over night. Water and diluted hydrochloric acid was added. The mixture was extracted with ethyl acetate, the organic phase was dried over magnesium sulfate and evaporated to give the title compound in 97% yield. MS (ESI) m/z 311, 313 [M−1]⁻.

### EXAMPLE 57

### 4-(Benzofuran-2-yl)-3-fluoro-N-(2-sulfamoylphenylsulfonyl)benzamide

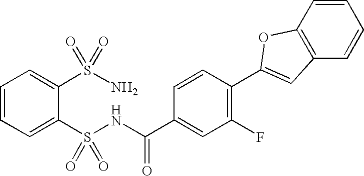

A mixture of 4-bromo-3-fluoro-N-(2-sulfamoylphenylsulfonyl)benzamide (0.18 g, 0.40 mmol), 2-benzofuranboronic acid (0.084 g, 0.52 mmol), 1,1′-bis(diphenylphosphino)ferrocene-palladium dichloride (0.033 mg, 0.040 mmol), N,N-dimethylformamide (3 mL) and sodium carbonate (2 M, 0.60 mL, 1.20 mmol) under an atmosphere of argon was heated in microwave at 120° C. for 0.5 h. The reaction mixture was partitioned between ethyl acetate and diluted hydrochloric acid and the organic phase was dried over magnesium sulfate and evaporated. The residue was purified by preparative HPLC, fractions containing the product was pooled, diluted hydrochloric acid was added and the mixture was extracted with dichloromethane. The organic phase was dried over magnesium sulfate and evaporated to give 0.059 g (31% yield) of the title compound. ¹H NMR (DMSO-d₆) δ ppm 8.30 (d, 1H) 8.09 (dd, 1H) 8.03 (t, 1H) 7.79-7.92 (m, 4H) 7.69 (d, 1H) 7.62 (dd, 1H) 7.30-7.51 (m, 4H) 7.20-7.30 (m, 1H); MS (APCI) m/z 473 [M−1]⁻.

### a) 4-Bromo-3-fluoro-N-(2-sulfamoylphenylsulfonyl)benzamide

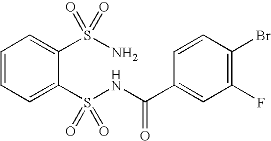

To a solution of benzene-1,2-disulfonamide (0.47 g, 2.00 mmol) and 4-bromo-3-fluorobenzoic acid (0.44 g, 2.00 mmol) in N,N-dimethylformamide (20 mL) was N-(3-dimethylaminopropyl)-N′-ethylcarbodiimide hydrochloride (0.58 g, 3.00 mmol) and 4-(dimethylamino)pyridine (0.37 g, 3.00 mmol) added, the resulting mixture was stirred at room temperature over night. Water was added and the mixture was washed with ethyl acetate. The aqueous phase was acidified by addition of 1 M hydrochloric acid and extracted with ethyl acetate. The organic phase was dried over magnesium sulfate and evaporated to give 0.77 g (88% yield) of the title compound. ¹H NMR (DMSO-d₆) δ ppm 8.30-8.37 (m, 1H) 8.14 (d, 1H) 7.78-7.94 (m, 4H) 7.66 (dd, 1H) 7.45 (br. s., 2H); MS (ESI) m/z 435, 437 [M−1]⁻.

### EXAMPLE 58

### 4-(3-Methylbut-1-ynyl)-N-(2-sulfamoylphenylsulfonyl)-3-(2,2,3,3-tetrafluoropropoxy)benzamide

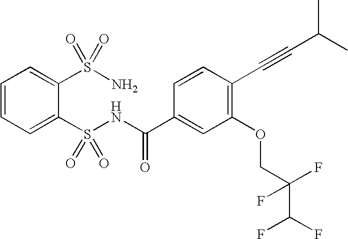

The title compound was synthesized as described for Example 43 in 39% yield, starting from 4-bromo-N-(2-sulfamoylphenylsulfonyl)-3-(2,2,3,3-tetrafluoropropoxy)benzamide and 3-methylbut-1-yne. Purification by preparative HPLC. ¹H NMR (DMSO-d₆) δ ppm 8.27 (dd, 1H) 8.04-8.13 (m, 1H) 7.75-7.90 (m, 2H) 7.57 (d, 1H) 7.31-7.51 (m, 4H) 6.35-6.68 (m, 1H) 4.63 (t, 2H) 2.71-2.84 (m, 1H) 1.14 (d, 6H); MS (APCI) m/z 535 [M−1]⁻.

### a) 4-Bromo-N-(2-sulfamoylphenylsulfonyl)-3-(2,2,3,3-tetrafluoropropoxy)benzamide

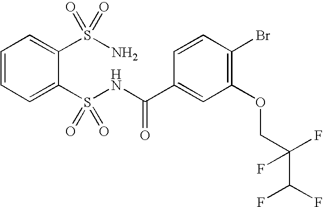

The title compound was synthesized as described for Example 46a) in 62% yield, starting from 4-bromo-3-(2,2,3,3-tetrafluoropropoxy)benzoic acid. ¹H NMR (DMSO-d₆) δ ppm 8.35 (d, 1H) 8.12-8.20 (m, 1H) 7.83-7.94 (m, 2H) 7.76 (d, 1H) 7.71 (d, 1H) 7.42-7.52 (m, 3H) 6.51-6.79 (m, 1H) 4.77 (t, 2H); MS (ESI) m/z 547, 549 [M−1]⁻.

### b) 4-Bromo-3-(2,2,3,3-tetrafluoropropoxy)benzoic acid

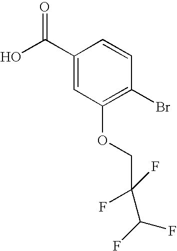

The title compound was synthesized as described for Example 48b) starting from methyl 4-bromo-3-(2,2,3,3-tetrafluoropropoxy)benzoate. ¹H NMR (DMSO-d₆) δ ppm 13.34 (br. s., 1H) 7.78 (d, 1H) 7.69 (d, 1H) 7.55 (dd, 1H) 6.50-6.76 (m, 1H) 4.83 (t, 2H); MS (ESI) m/z 329, 331 [M−1]⁻.

### c) Methyl 4-bromo-3-(2,2,3,3-tetrafluoropropoxy)benzoate

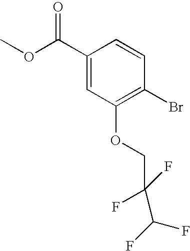

The title compound was synthesized as described for Example 53c) using 2,2,3,3-tetrafluoropropyl p-toluenesulfonate. ¹H NMR (DMSO-d₆) δ ppm 7.86 (d, 1H) 7.80 (d, 1H) 7.70 (d, 1H) 6.50-6.75 (m, 1H) 4.84 (t, 2H) 3.87 (s, 3H); GC MS (EI) m/z 344, 346 [M]^{+•}.

### EXAMPLE 59

### 4-(Benzofuran-2-yl)-N-(2-sulfamoylphenylsulfonyl)-3-(3,3,3-trifluoropropoxy)benzamide

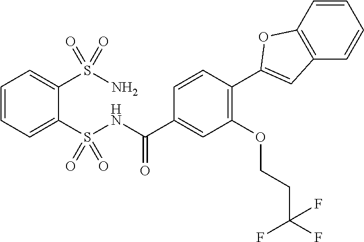

The title compound was synthesized as described for Example 57 in 26% yield, starting from 4-bromo-N-(2-sulfamoylphenylsulfonyl)-3-(3,3,3-trifluoropropoxy)benzamide and heating the reaction at 140° C. Purification by preparative HPLC. ¹H NMR (DMSO-d₆) δ ppm 8.34 (br. s., 1H) 8.14 (br. s., 1H) 8.04 (d, 1H) 7.86 (br. s., 2H) 7.76 (s, 1H) 7.70 (d, 1H) 7.65 (t, 2H) 7.56 (s, 1H) 7.45 (s, 2H) 7.37 (t, 1H) 7.28 (t, 1H) 4.50 (t, 2H) 3.04-3.14 (m, 2H); MS (APCI) m/z 567 [M−1]⁻.

### EXAMPLE 60

### 4-(3,3-Dimethylbut-1-ynyl)-N-(2-sulfamoylphenylsulfonyl)-3-(3,3,3-trifluoropropoxy)benzamide

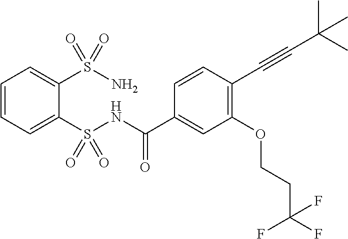

The title compound was synthesized as described for Example 44 in 35% yield, starting from 4-bromo-N-(2-sulfamoylphenylsulfonyl)-3-(3,3,3-trifluoropropoxy)benzamide and 3,3-dimethylbut-1-yne and heating the reaction for 3 h. Purification by preparative HPLC. ¹H NMR (DMSO-d₆) δ ppm 8.33 (d, 1H) 8.14 (d, 1H) 7.81-7.91 (m, 2H) 7.55 (s, 1H) 7.31-7.46 (m, 4H) 4.28 (t, 2H) 2.77-2.90 (m, 2H) 1.28 (s, 9H); MS (ESI) m/z 531 [M−1]⁻.

### EXAMPLE 61

### 4-(3,3-Dimethylbut-1-ynyl)-N-(2-sulfamoylphenylsulfonyl)-3-(2-(2,2,2-trifluoroethoxy)ethoxy)benzamide

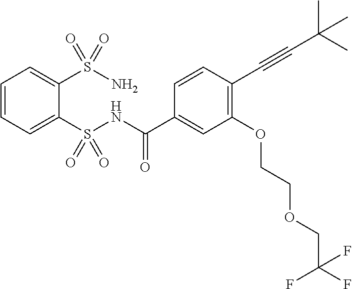

The title compound was synthesized as described for Example 44 in 30% yield, starting from 4-bromo-N-(2-sulfamoylphenylsulfonyl)-3-(2-(2,2,2-trifluoroethoxy)ethoxy)benzamide and 3,3-dimethylbut-1-yne and heating the reaction for 3 h. Purification by preparative HPLC. ¹H NMR (DMSO-d₆) δ ppm 8.35 (d, 1H) 8.13-8.17 (m, 1H) 7.84-7.95 (m, 2H) 7.53 (d, 1H) 7.33-7.46 (m, 4H) 4.17-4.28 (m, 4H) 3.96-4.04 (m, 2H) 1.28 (s, 9H); MS (APCI) m/z 561 [M−1]⁻.

### EXAMPLE 62

### 4-(Benzofuran-2-yl)-N-(2-sulfamoylphenylsulfonyl)-3-(2-(2,2,2-trifluoroethoxy)ethoxy)benzamide

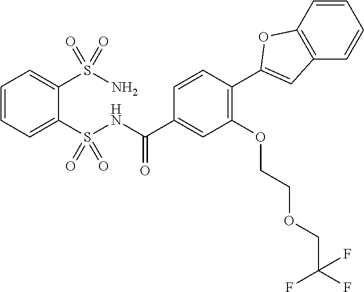

The title compound was synthesized as described for Example 57 in 32% yield, starting from 4-bromo-N-(2-sulfamoylphenylsulfonyl)-3-(2-(2,2,2-trifluoroethoxy)ethoxy)benzamide and heating the reaction at 140° C. Purification by preparative HPLC. ¹H NMR (DMSO-d₆) δ ppm 8.37 (br. s., 1H) 8.13-8.19 (m, 1H) 8.04 (d, 1H) 7.85-7.94 (m, 2H) 7.73 (s, 1H) 7.61-7.69 (m, 4H) 7.45 (s, 2H) 7.33-7.41 (m, 1H) 7.29 (t, 1H) 4.40-4.47 (m, 2H) 4.28 (q, 2H) 4.09-4.19 (m, 2H); MS (APCI) m/z 597 [M−1]⁻.

### EXAMPLE 63

### 4-(Phenylethynyl)-N-(2-sulfamoylphenylsulfonyl)-3-(2-(2,2,2-trifluoroethoxy)ethoxy)benzamide

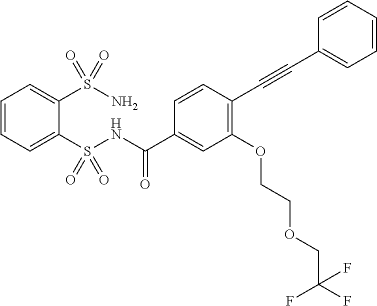

The title compound was synthesized as described for Example 43 in 27% yield, starting from 4-bromo-N-(2-sulfamoylphenylsulfonyl)-3-(2-(2,2,2-trifluoroethoxy)ethoxy)benzamide and phenylacetylene. Purification by preparative HPLC. ¹H NMR (DMSO-d₆) δ ppm 8.31-8.37 (m, 1H) 8.12-8.17 (m, 1H) 7.82-7.92 (m, 2H) 7.61 (s, 1H) 7.48-7.59 (m, 4H) 7.40-7.47 (m, 5H) 4.19-4.33 (m, 4H) 3.99-4.09 (m, 2H); MS (APCI) m/z 581 [M−1]⁻.

### EXAMPLE 64

### 4-(3-Methylbut-1-ynyl)-N-(2-sulfamoylphenylsulfonyl)-3-(4,4,4-trifluorobutoxy)benzamide

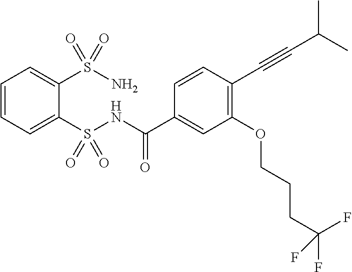

The title compound was synthesized as described for Example 43 in 26% yield, starting from 4-bromo-N-(2-sulfamoylphenylsulfonyl)-3-(4,4,4-trifluorobutoxy)benzamide and 3-methylbut-1-yne. Purification by preparative HPLC. ¹H NMR (CDCl₃) δ ppm 9.36 (s, 1H) 8.43-8.54 (m, 1H) 8.16-8.27 (m, 1H) 7.70-7.81 (m, 2H) 7.33 (d, 1H) 7.23 (dd, 1H) 7.17 (d, 1H) 5.61 (s, 2H) 3.99 (t, 2H) 2.69-2.80 (m, 1H) 2.25-2.37 (m, 2H) 1.97-2.05 (m, 2H) 1.19 (d, 6H); MS (APCI) m/z 531 [M−1]⁻.

### a) 4-Bromo-N-(2-sulfamoylphenylsulfonyl)-3-(4,4,4-trifluorobutoxy)benzamide

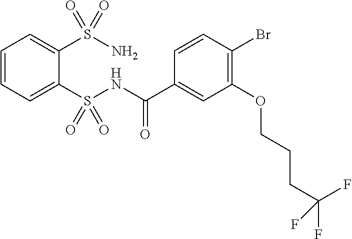

The title compound was synthesized as described for Example 46a) starting from 4-bromo-3-(4,4,4-trifluorobutoxy)benzoic acid. ¹H NMR (DMSO-d₆) δ ppm 8.35 (dd, 1H) 8.16 (dd, 1H) 7.85-7.95 (m, 2H) 7.71 (d, 1H) 7.60 (d, 1H) 7.39-7.49 (m, 3H) 4.20 (t, 2H) 2.41-2.49 (m, 2H) 1.96-2.06 (m, 2H); MS (ESI) m/z 543, 545 [M−1]⁻.

### b) 4-Bromo-3-(4,4,4-trifluorobutoxy)benzoic acid

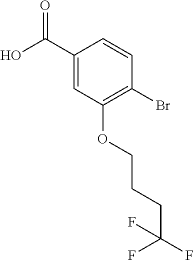

The title compound was synthesized as described for Example 48b) starting from methyl 4-bromo-3-(4,4,4-trifluorobutoxy)benzoate. ¹H NMR (DMSO-d₆) δ ppm 13.22 (s, 1H) 7.73 (d, 1H) 7.54 (d, 1H) 7.47 (dd, 1H) 4.20 (t, 2H) 2.42-2.49 (m, 2H) 1.96-2.04 (m, 2H); MS (ESI) m/z 325, 327 [M−1]⁻.

### c) Methyl 4-bromo-3-(4,4,4-trifluorobutoxy)benzoate

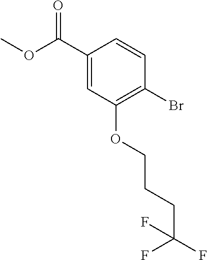

The title compound was synthesized as described for Example 48c) in 91% yield, starting from 4,4,4-trifluorobutanol. Purification by column chromatography, using a gradient of ethyl acetate (0-30%) in heptane as the eluent. ¹H NMR (DMSO-d₆) δ ppm 7.76 (d, 1H) 7.54 (d, 1H) 7.49 (dd, 1H) 4.21 (t, 2H) 3.86 (s, 3H) 2.41-2.48 (m, 2H) 1.94-2.06 (m, 2H); GC MS (EI) m/z 340, 342 [M]^{+•}.

### EXAMPLE 65

### 4-(Phenylethynyl)-N-(2-sulfamoylphenylsulfonyl)-3-(3,3,3-trifluoropropoxy)benzamide

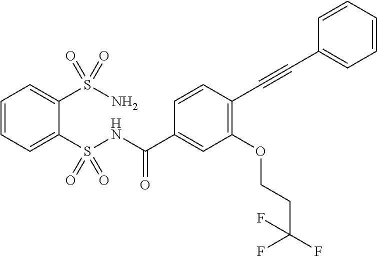

The title compound was synthesized as described for Example 43 in 19% yield, starting from 4-bromo-N-(2-sulfamoylphenylsulfonyl)-3-(3,3,3-trifluoropropoxy)benzamide and phenylacetylene. Purification by preparative HPLC. ¹H NMR (DMSO-d₆) δ ppm 8.35 (d, 1H) 8.15 (d, 1H) 7.82-7.94 (m, 2H) 7.63 (s, 1H) 7.48-7.60 (m, 4H) 7.40-7.49 (m, 5H) 4.37 (t, 2H) 2.84-2.98 (m, 2H); MS (APCI) m/z 552 [M−1]⁻.

### EXAMPLE 66

### 4-(Cyclopentylethynyl)-N-(2-sulfamoylphenylsulfonyl)-3-(3,3,3-trifluoropropoxy)benzamide

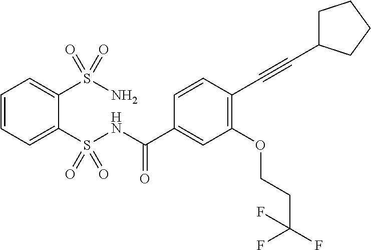

The title compound was synthesized as described for Example 43 in 26% yield, starting from 4-bromo-N-(2-sulfamoylphenylsulfonyl)-3-(3,3,3-trifluoropropoxy)benzamide and cyclopentylacetylene. Purification by preparative HPLC. ¹H NMR (DMSO-d₆) δ ppm 8.33 (d, 1H) 8.14 (d, 1H) 7.81-7.95 (m, 2H) 7.55 (s, 1H) 7.33-7.47 (m, 4H) 4.29 (t, 2H) 2.75-2.94 (m, 3H) 1.88-2.00 (m, 2H) 1.51-1.75 (m, 6H); MS (APCI) m/z 543 [M−1]⁻.

### EXAMPLE 67

### 4-(Cyclopentylethynyl)-N-(2-sulfamoylphenylsulfonyl)-3-(2-(2,2,2-trifluoroethoxy)ethoxy)benzamide

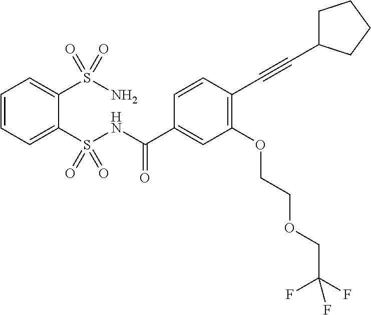

The title compound was synthesized as described for Example 43 in 15% yield, starting from 4-bromo-N-(2-sulfamoylphenylsulfonyl)-3-(2-(2,2,2-trifluoroethoxy)ethoxy)benzamide and cyclopentylacetylene. Purification by preparative HPLC. ¹H NMR (CDCl₃) δ ppm 9.44 (s, 1H) 8.51-8.60 (m, 1H) 8.26-8.34 (m, 1H) 7.80-7.89 (m, 2H) 7.30-7.45 (m, 2H) 5.69 (s, 2H) 4.16-4.23 (m, 2H) 4.01-4.13 (m, 4H) 2.86 (quin, 1H) 1.95-2.07 (m, 2H) 1.59-1.83 (m, 6H); MS (APCI) m/z 573 [M−1]⁻

### EXAMPLE 68

### 4-(Cyclopentylethynyl)-3-(3,3-difluoropropoxy)-N-(2-sulfamoylphenylsulfonyl)benzamide

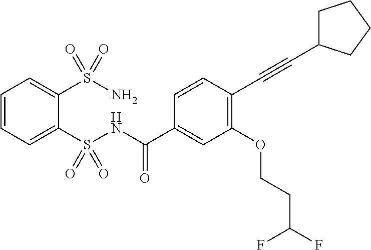

The title compound was synthesized as described for Example 43 in 26% yield, starting from 4-bromo-3-(3,3-difluoropropoxy)-N-(2-sulfamoylphenylsulfonyl)benzamide and cyclopentylacetylene. Purification by preparative HPLC. ¹H NMR (CDCl₃) δ ppm 9.45 (s, 1H) 8.58 (dd, 1H) 8.31 (dd, 1H) 7.82-7.89 (m, 2H) 7.32-7.44 (m, 2H) 6.05-6.28 (m, 1H) 5.70 (s, 2H) 4.19 (t, 2H) 2.90 (quin, 1H) 2.32-2.42 (m, 2H) 1.61-2.07 (m, 8H); MS (APCI) m/z 525 [M−1]⁻.

### a) 4-Bromo-3-(3,3-difluoropropoxy)-N-(2-sulfamoylphenylsulfonyl)benzamide

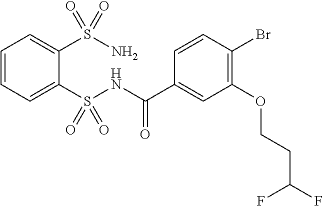

The title compound was synthesized as described for Example 46a) starting from 4-bromo-3-(3,3-difluoropropoxy)benzoic acid. MS (ESI) m/z 511, 513 [M−1]⁻.

### b) 4-Bromo-3-(3,3-difluoropropoxy)benzoic acid

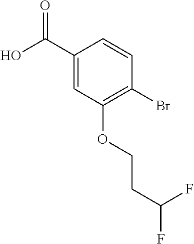

The title compound was synthesized as described for Example 48b) in 91% yield, starting from methyl 4-bromo-3-(3,3-difluoropropoxy)benzoate. ¹H NMR (DMSO-d₆) δ ppm 13.23 (br. s., 1H) 7.74 (d, 1H) 7.57 (s, 1H) 7.49 (d, 1H) 6.13-6.40 (m, 1H) 4.29 (t, 2H) 2.29-2.45 (m, 2H); MS (ESI) m/z 293, 295 [M−1]⁻.

### c) Methyl 4-bromo-3-(3,3-difluoropropoxy)benzoate

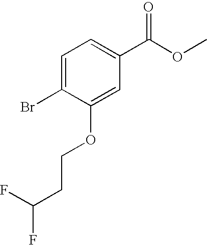

Bis(2-methoxyethyl)amino-sulfur trifluoride (0.25 mL, 1.35 mmol) was added dropwise to a cooled (0° C.) solution of methyl 4-bromo-3-(3-oxopropoxy)benzoate (0.13 g, 0.45 mmol) in dichloromethane (5 mL). The reaction was stirred for 1 h and partitioned is between saturated sodium bicarbonate and dichloromethane. The organic phase was dried over magnesium sulfate and evaporated. Purification by column chromatography, using heptane/ethyl acetate (4:1) as the eluent, gave 32% yield of the title compound. ¹H NMR (DMSO-d₆) δ ppm 7.77 (d, 1H) 7.57 (d, 1H) 7.50 (dd, 1H) 6.15-6.38 (m, 1H) 4.29 (t, 2H) 3.86 (s, 3H) 2.33-2.44 (m, 2H); GC MS (EI) m/z 308, 310 [M]^{+•}.

### d) Methyl 4-bromo-3-(3-oxopropoxy)benzoate

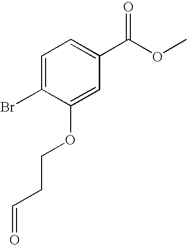

Dess-Martin periodinane (0.29 g, 0.68 mmol) was added to a solution of methyl 4-bromo-3-(3-hydroxypropoxy)benzoate (0.13 g, 0.45 mmol) and tert-butanol (0.064 mL, 0.68 mmol) in dichloromethane (5 mL) at 0° C. and the resulting mixture was stirred for 1.5 h. The reaction mixture was partitioned between a mixture of saturated sodium thiosulfate and saturated sodium bicarbonate and dichloromethane, the organic phase was dried over magnesium sulfate and evaporated. GC MS (EI) m/z 286, 288 [M]^{+•}.

### e) Methyl 4-bromo-3-(3-hydroxypropoxy)benzoate

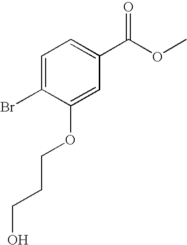

Methyl 4-bromo-3-(3-(tert-butyldimethylsilyloxy)propoxy)benzoate (1.86 g, 4.61 mmol) is was treated with tetra-n-butylammonium fluoride (1 M in tetrahydrofuran, 9.22 mL1, 9.22 mmol) for 3 days. Purification by column chromatography, using a gradient of ethyl acetate (0-100%) in heptane as the eluent, gave 54% yield of the title compound. ¹H NMR (DMSO-d₆) δ ppm 7.74 (d, 1H) 7.54 (d, 1H) 7.46 (dd, 1H) 4.57 (t, 1H) 4.18 (t, 2H) 3.86 (s, 3H) 3.60 (q, 2H) 1.90 (quin, 2H); MS (ESI) m/z 289, 291 [M+1]⁺.

### f) Methyl 4-bromo-3-(3-(tert-butyldimethylsilyloxy)propoxy)benzoate

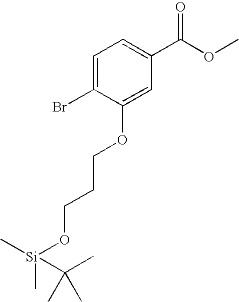

A solution of methyl 4-bromo-3-hydroxybenzoate (2.31 g, 10.0 mmol) in N,N-dimethylformamide (10 mL) was added dropwise to a slurry of sodium hydride (60% in mineral oil, 0.48 g, 12.0 mmol) in N,N-dimethylformamide (30 mL) at 0° C. The reaction was stirred for 1 h and then (3-bromopropoxy)-tert-butyldimethylsilane (2.55 mL, 11.0 mmol) was added dropwise. The reaction was stirred for 3 h at room temperature, diluted with toluene and washed with diluted hydrochloric acid. The organic phase was dried over to magnesium sulfate and evaporated. Purification by column chromatography using dichloromethane, as the eluent gave 46% yield of the title compound. ¹H NMR (DMSO-d₆) δ ppm 7.74 (d, 1H) 7.53 (d, 1H) 7.46 (dd, 1H) 4.18 (t, 2H) 3.85 (s, 3H) 3.80 (t, 2H) 1.90-2.00 (m, 2H) 0.84 (s, 9H) 0.01 (s, 6H); MS (ESI) m/z 403, 405 [M+1]⁺.

### EXAMPLE 69

### 4-(Cyclopentylethynyl)-N-(2-sulfamoylphenylsulfonyl)-3-(4,4,4-trifluorobutoxy)benzamide

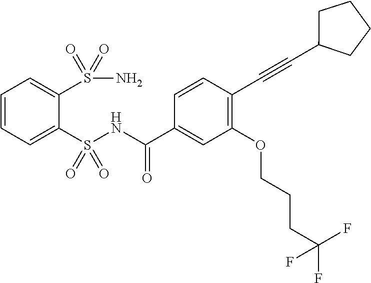

The title compound was synthesized as described for Example 43 in 28% yield, starting from 4-bromo-N-(2-sulfamoylphenylsulfonyl)-3-(4,4,4-trifluorobutoxy)benzamide and cyclopentylacetylene. Purification by preparative HPLC. ¹H NMR (CDCl₃) δ ppm 9.45 (s, 1H) 8.57 (dd, 1H) 8.28-8.34 (m, 1H) 7.80-7.92 (m, 2H) 7.30-7.44 (m, 2H) 7.26 (d, 1H) 5.71 (s, 2H) 4.08 (t, 2H) 2.90 (quin, 1H) 2.34-2.45 (m, 2H) 1.98-2.15 (m, 4H) 1.60-1.85 (m, 6H); MS (APCI) m/z 557 [M−1]⁻.

### EXAMPLE 70

### 3-(3,3-Difluoropropoxy)-N-(2-sulfamoylphenylsulfonyl)-4-(p-tolylethynyl)benzamide

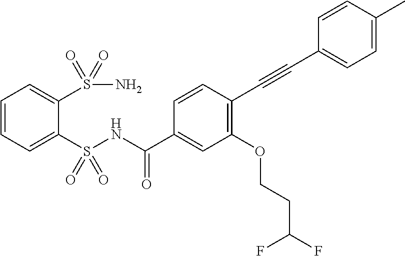

The title compound was synthesized as described for Example 43 in 16% yield, starting from 4-bromo-3-(3,3-difluoropropoxy)-N-(2-sulfamoylphenylsulfonyl)benzamide and 1-ethynyl-4-methylbenzene. Purification by preparative HPLC. ¹H NMR (DMSO-d₆) δ ppm 8.34 (d, 1H) 8.14 (d, 1H) 7.80-7.94 (m, 2H) 7.38-7.65 (m, 7H) 7.25 (d, 2H) 6.14-6.43 (m, 1H) 4.28 (t, 2H) 2.37-2.47 (m, 2H) 2.34 (s, 3H); MS (APCI) m/z 547 [M−1]⁻.

### a) 3-(3,3-Difluoropropoxy)-4-(3-methylbut-1-ynyl)-N-(2-sulfamoylphenylsulfonyl)benzamide and 3-(3-Fluoropropoxy)-4-(3-methylbut-1-ynyl)-N-(2-sulfamoylphenylsulfonyl)benzamide

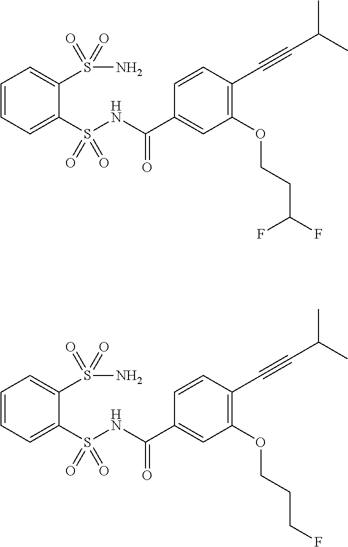

The title compounds were synthesized as described for Example 43 starting from a mixture of 4-bromo-3-(3,3-difluoropropoxy)-N-(2-sulfamoylphenylsulfonyl)benzamide and 4-bromo-3-(3-fluoropropoxy)-N-(2-sulfamoylphenylsulfonyl)benzamide and 3-methylbut-1-yne. Extractive work-up gave the title compounds as a 7:3 mixture. MS (APCI) m/z 499 [M−1]⁻ and MS (APCI) m/z 481 [M−1]⁻.

### b) 4-Bromo-3-(3,3-difluoropropoxy)-N-(2-sulfamoylphenylsulfonyl)benzamide and 4-Bromo-3-(3-fluoropropoxy)-N-(2-sulfamoylphenylsulfonyl)benzamide

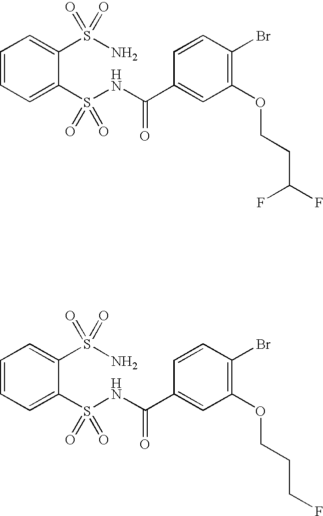

The title compounds were synthesized as described for Example 46a) starting from a mixture of 4-bromo-3-(3,3-difluoropropoxy)benzoic acid and 4-bromo-3-(3-fluoropropoxy)benzoic acid. Extractive work-up gave the crude title compounds as a 7:3 mixture. MS (ESI) m/z 511, 513 [M−1]⁻ and MS (ESI) m/z 493, 495 [M−1]⁻.

### c) 4-Bromo-3-(3,3-difluoropropoxy)benzoic acid and 4-Bromo-3-(3-fluoropropoxy)benzoic acid

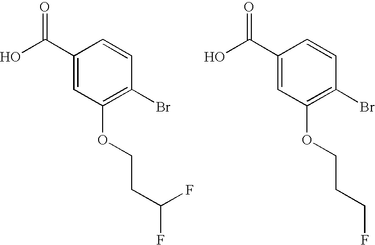

The title compounds were synthesized as described for Example 48b) starting from a mixture of methyl 4-bromo-3-(3,3-difluoropropoxy)benzoate and methyl 4-bromo-3-(3-fluoropropoxy)benzoate. Extractive work-up gave the title compounds as a crude mixture. MS (ESI) m/z 293, 295 [M−1]⁻ and MS (ESI) m/z 277, 275 [M−1]⁻.

### d) Methyl 4-bromo-3-(3,3-difluoropropoxy)benzoate and Methyl 4-bromo-3-(3-fluoropropoxy)benzoate

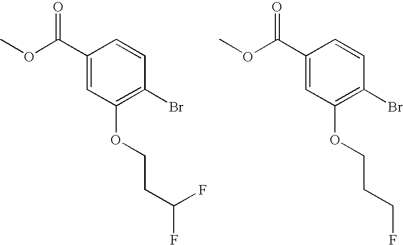

The title compounds were synthesized as described in Examples 68c) and d) starting from methyl 4-bromo-3-(3-hydroxypropoxy)benzoate. Purification by column chromatography gave the title compounds as a mixture. GC MS (EI) m/z 308, 310 [M]^{+•} and GC MS (EI) m/z 290, 292 [M]^{+•}.

### EXAMPLE 71

### 3-(3,3-Difluoropropoxy)-4-(3-methylbut-1-ynyl)-N-(2-sulfamoylphenylsulfonyl)benzamide

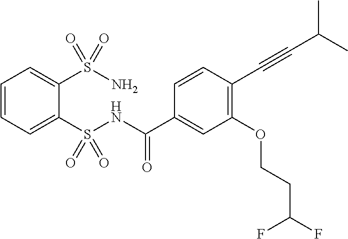

The title compound was obtained after separation of the product mixture obtained in Example 70a) by preparative HPLC. ¹H NMR (CDCl₃) δ ppm 9.45 (s, 1H) 8.56-8.60 (m, 1H) 8.31 (dd, 1H) 7.82-7.89 (m, 2H) 7.31-7.73 (m, 3H) 6.05-6.28 (m, 1H) 5.71 (s, 2H) 4.19 (t, 2H) 2.79-2.88 (m, 1H) 2.31-2.43 (m, 2H) 1.29 (d, 6H); MS (APCI) m/z 499 [M−1]⁻.

### EXAMPLE 72

### 3-(3-Fluoropropoxy)-4-(3-methylbut-1-ynyl)-N-(2-sulfamoylphenylsulfonyl)benzamide

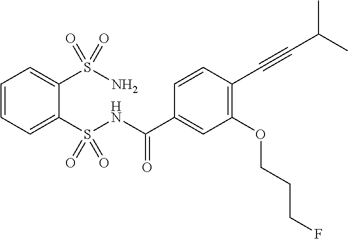

The title compound was obtained after separation of the product mixture obtained in Example 70a) by preparative HPLC. ¹H NMR (CDCl₃) δ ppm 9.34 (s, 1H) 8.45-8.50 (m, 1H) 8.20 (dd, 1H) 7.71-7.79 (m, 2H) 7.19-7.33 (m, 3H) 5.60 (s, 2H) 4.54-4.67 (m, 2H) 4.06 (t, 2H) 2.70-2.78 (m, 1H) 2.07-2.17 (m, 2H) 1.18 (d, 6H); MS (APCI) m/z 481 [M−1]⁻.

### EXAMPLE 73

### 3-Methoxy-4-(phenylethynyl)-N-(2-sulfamoylphenylsulfonyl)benzamide

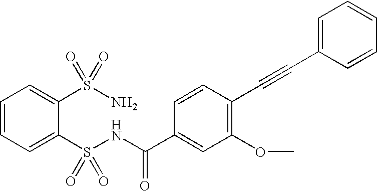

Ethynylbenzene (0.110 mL, 1.00 mmol), tetrakis(triphenylphosphine)palladium(0) (77 mg, 0.07 mmol) and triethylamine (2.78 mL, 20.03 mmol) was added to a solution of 4-bromo-3-methoxy-N-(2-sulfamoylphenylsulfonyl)benzamide (300 mg, 0.67 mmol) in N,N-dimethylformamide (13 mL) under an atmosphere of argon. The reaction mixture was stirred at room temperature for 5 min, copper(I) iodide (19.07 mg, 0.10 mmol) was added and the reaction mixture was heated at 75° C. for 3 days. The crude was filtered through a plug of celite and concentrated in vacuo. Purification by preparative HPLC gave 120 mg (38% yield) of the title compound. ¹H NMR (500 MHz, DMSO-d₆) δ ppm 8.14 (dd, 1H) 8.00 (dd, 1H) 7.62-7.67 (m, 1H) 7.56-7.61 (m, 1H) 7.49-7.55 (m, 4H) 7.40-7.47 (m, 6H) 7.08 (br. s., 1H) 3.85 (s, 3H); MS (ESI) m/z 469 [M−1]⁻

### EXAMPLE 74

### 3-Methoxy-4-(3-methylbut-1-ynyl)-N-(2-sulfamoylphenylsulfonyl)benzamide

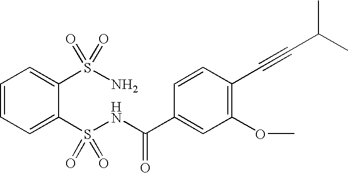

3-Methylbut-1-yne (68.2 mg, 1.00 mmol), tetrakis(triphenylphosphine)palladium(0) (77 mg, 0.07 mmol) and triethylamine (2.78 mL, 20.03 mmol) were added to a solution of 4-bromo-3-methoxy-N-(2-sulfamoylphenylsulfonyl)benzamide (300 mg, 0.67 mmol) in N,N-dimethylformamide (13 mL) under an atmosphere of argon. The reaction mixture was stirred at room temperature for 5 min, copper(I) iodide (19.07 mg, 0.10 mmol) was added and the reaction mixture was heated at 75° C. for 3 days. The crude was filtered through a plug of celite and concentrated in vacuo. Purification by preparative HPLC gave 79 mg (27% yield) of the title compound. ¹H NMR (500 MHz, DMSO-d₆) δ ppm 8.13 (d, 1H) 7.99 (d, 1H) 7.64 (t, 1H) 7.58 (t, 1H) 7.35-7.53 (m, 3H) 7.24 (d, 1H) 3.78 (s, 3H) 2.70-2.93 (m, 1H) 1.20 (d, 6H); MS (ESI) m/z 435 [M−1]⁻.

### EXAMPLE 75 and 76

### 4-Hydroxy-benzene-1,2-disulfonic acid 2-amide 1-(4-benzofuran-2-yl-benzoylamide) (75) and 4-methoxy-benzene-1,2-disulfonic acid 2-amide 1-(4-benzofuran-2-yl-benzoylamide) (76)

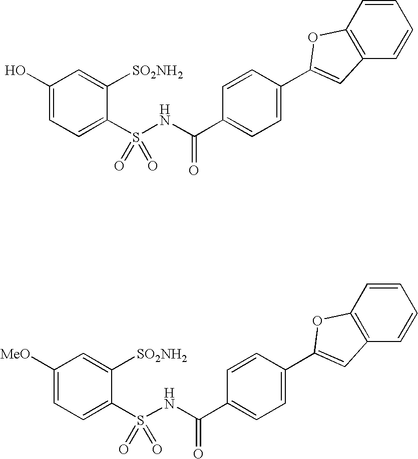

Boron tribromide (1 M in dichloromethane, 8.6 mL, 8.59 mmol) was added dropwise to a suspension of 4-methoxy-benzene-1,2-disulfonic acid 1-(4-benzofuran-2-yl-benzoylamide) 2-tert-butylamide (0.23 g, 0.43 mmol) in dichloromethane (15 mL) at −20° C. The reaction mixture was allowed to warm to room temperature and stirred overnight. The reaction mixture was then cooled to −20° C., methanol (10 mL) was added slowly and the volatiles were removed under reduced pressure. The residue was taken up in methanol (10 mL), the mixture was concentrated in vacuo and the process was repeated once more. Purification by preparative HPLC gave 40 mg of 4-hydroxy-benzene-1,2-disulfonic acid 2-amide 1-(4-benzofuran-2-yl-benzoylamide) and 41.3 mg of 4-methoxy-benzene-1,2-disulfonic acid 2-amide 1-(4-benzofuran-2-yl-benzoylamide).

4-Hydroxy-benzene-1,2-disulfonic acid 2-amide 1-(4-benzofuran-2-yl-benzoylamide) (75): ¹H NMR (400 MHz, CD₃OD) ppm δ 8.23 (d, 1H) 8.03 (d, 2H) 7.93 (d, 2H) 7.59-7.66 (m, 2H) 7.54 (d, 1H) 7.28-7.36 (m, 2H) 7.19-7.28 (m, 1H) 7.04 (dd, 1H); MS (ESI) m/z 470.7 [M−1]⁻.

4-Methoxy-benzene-1,2-disulfonic acid 2-amide 1-(4-benzofuran-2-yl-benzoylamide) (76): ¹H NMR (400 MHz, CD₃OD) ppm δ 8.27 (d, 1H) 8.08 (d, 2H) 7.89 (d, 2H) 7.68 (d, 1H) 7.61 (d, 1H) 7.53 (d, 1H) 7.22-7.34 (m, 3H) 7.19 (dd, 1H) 3.89 (s, 3H); MS (ESI) m/z 484.8 [M−1]⁻.

### a) 4-Methoxy-benzene-1,2-disulfonic acid 1-(4-benzofuran-2-yl-benzoylamide) 2-tert-butylamide

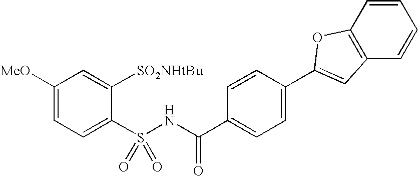

To a degassed solution of 4-methoxy-benzene-1,2-disulfonic acid 1-(4-bromo-benzoylamide) 2-tert-butylamide (0.80 g, 1.58 mmol) in dimethoxyethane (32 mL) was added a solution of benzo[b]furan-2-boronic acid (0.36 g, 2.22 mmol) in a mixture of dimethoxyethane (7.2 mL) and ethanol (2.4 mL) followed by sodium bicarbonate (2 M aqueous, 4.8 mL, 9.50 mmol) and [1,1′-bis(diphenylphosphino)ferrocene]dichloropalladium (dichloromethane complex) (0.13 g, 0.16 mmol). The reaction mixture was degassed for 15 minutes and heated in a sealed tube at 100° C. overnight. The reaction mixture was cooled to room temperature, filtered through a pad of Celite and concentrated under reduced pressure. Purification by column chromatography, using a gradient of 30-70% ethyl acetate in hexane as the eluent, gave 397 mg (46% yield) of the title compound. ¹H NMR (400 MHz, DMSO-d₆) δ ppm 8.26 (d, 1H) 7.99 (s, 4H) 7.54-7.71 (m, 4H) 7.32-7.40 (m, 2H) 7.24-7.31 (m, 1H) 7.16 (br. s., 1H) 3.90 (s, 3H) 1.11-1.19 (m, 9H); MS (ESI) m/z 541 [M−1]⁻.

### b) 4-Methoxy-benzene-1,2-disulfonic acid 1-(4-bromo-benzoylamide) 2-tert-butylamide

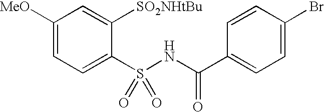

A mixture of 2-tert-butylsulfamoyl-4-methoxy-benzenesulfonamide (1.18 g, 3.66 mmol), 4-bromobenzoic acid (0.74 g, 3.66 mmol), N-(3-dimethylaminopropyl)-N′-ethylcarbodiimide hydrochloride (0.70 g, 3.66 mmol) and 4-(dimethylamino)pyridine (0.45 g, 3.66 mmol) in anhydrous dichloromethane (300 mL) was stirred at room temperature for 2.5 days. The reaction mixture was concentrated under reduced pressure and the residue was purified by column chromatography, using dichloromethane as the eluent, to give 1.68 g (91% yield) of the title compound. ¹H NMR (400 MHz, DMSO-d₆) δ ppm 8.26 (d, 1H) 7.79 (d, 2H) 7.69 (d, 2H) 7.59 (d, 1H) 7.08 (s, 1H) 3.90 (s, 3H) 1.13 (s, 9H); MS (ESI) m/z 503, 505 [M−1]⁻.

### c) 2-tert-Butylsulfamoyl-4-methoxy-benzenesulfonamide

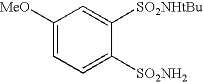

Ammonium hydroxide (28% aqueous solution, 1.1 mL, 7.55 mmol) was added to a solution of 2-tert-butylsulfamoyl-4-methoxy-benzenesulfonyl chloride (1.29 g, 3.77 mmol) in tetrahydrofuran (60 mL) at room temperature. The reaction mixture was stirred for 1 hour and then filtered to remove the precipitated ammonium chloride. The filtrate was concentrated under reduced pressure to give 1.22 g (quantitative yield) of the title compound. ¹H NMR (400 MHz, DMSO-d₆) δ ppm 8.08 (d, 1H) 7.57 (d, 1H) 7.35 (dd, 1H) 7.18 (s, 2H) 6.81 (s, 1H) 3.89 (s, 3H) 1.13 (s, 9H).

### d) 2-tert-Butylsulfamoyl-4-methoxy-benzenesulfonyl chloride

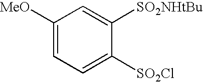

Water (250 mL) and formic acid (250 mL) were added to a solution of 2-benzylsulfanyl-N-tert-butyl-5-methoxy-benzenesulfonamide (5.57 g, 15.24 mmol) in dichloromethane (500 mL). The reaction mixture was cooled to 0° C. and chlorine gas was bubbled through the heterogeneous mixture with vigorous stirring for 15 minutes. The reaction mixture was then warmed to room temperature and stirred for 30 minutes under a nitrogen flow to remove excess chlorine gas. Dichloromethane (500 mL) was added and the phases were separated. The organic phase was washed with brine, dried over magnesium sulfate and concentrated under reduced pressure. Purification by column chromatography, using 15% ethyl acetate in hexane as the eluent, followed by recrystallized from a mixture of hexane and dichloromethane gave 1.29 g (35% yield) of the title compound. ¹H NMR (400 MHz, CDCl₃) δ ppm 8.27 (d, 1H) 7.85 (d, 1H) 7.15 (dd, 1H) 5.79 (s, 1H) 3.99 (s, 3H) 1.26 (s, 9H).

### e) 2-Benzylsulfanyl-N-tert-butyl-5-methoxy-benzenesulfonamide

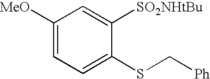

Tris(dibenzylideneacetone)dipalladium (0.37 g, 0.40 mmol), 4,5-bis(diphenylphosphino)-9,9-dimethylxanthene (0.46 g, 0.80 mmol) and benzyl mercaptan (1.9 mL, 15.95 mmol) were added to a degassed solution of 2-bromo-N-tert-butyl-5-methoxy-benzenesulfonamide (5.14 g, 15.95 mmol) and N,N-diisopropylethylamine (5.6 mL, 31.90 mmol) in anhydrous 1,4-dioxane (80 mL). The resulting mixture was degassed with nitrogen for 15 minutes and heated in a sealed tube at 100° C. overnight. Volatiles were removed under reduced pressure and the residue was purified by column chromatography, using a gradient of 0-15% ethyl acetate in hexane as the eluent, to give 5.67 g (97% yield) of the title compound. ¹H NMR (400 MHz, CDCl₃) δ ppm 7.66 (d, 1H) 7.16-7.29 (m, 6H) 6.85 (dd, 1H) 5.79 (s, 1H) 4.15 (s, 2H) 3.84 (s, 3H) 1.23 (s, 3H) 1.17 (s, 6H).

### f) 2-Bromo-N-tert-butyl-5-methoxy-benzenesulfonamide

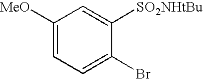

A solution of 2-bromo-5-methoxy-benzenesulfonyl chloride (4.94 g, 17.30 mmol) in anhydrous dichloromethane (100 mL) was added to a stirred mixture of tert-butylamine (3.6 mL, 34.60 mmol) and triethylamine (4.8 mL, 34.60 mmol) in anhydrous dichloromethane (100 mL) at room temperature. The reaction mixture was stirred at room temperature overnight and quenched with hydrochloric acid (2 M, 200 mL). The organic phase was separated, washed with brine, dried over magnesium sulfate and concentrated under reduced pressure to give 5.16 g (93% yield) of the title compound. ¹H NMR (400 MHz, CDCl₃) δ ppm 7.71 (d, 1H) 7.57 (d, 1H) 6.92 (dd, 1H) 5.15 (s, 1H) 3.85 (s, 3H) 1.23 (s, 9H).

### g) 2-Bromo-5-methoxy-benzenesulfonyl chloride

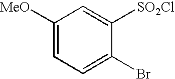

Water (250 mL) and formic acid (250 mL) were added to a solution of 2-benzylsulfanyl-1-bromo-4-methoxy-benzene (13.09 g, 42.33 mmol) in dichloromethane (500 mL). The reaction mixture was cooled to 0° C. and chlorine gas was bubbled through the heterogeneous mixture with vigorous stirring for 30 minutes. The reaction mixture was warmed to room temperature and stirred for 30 minutes under a nitrogen flow to remove excess chlorine gas. dichloromethane was added and the phases were separated. The organic phase was washed with brine, dried over magnesium sulfate and concentrated under reduced pressure. Purification by column chromatography, using a gradient of 2-4% diethyl ether in hexane as the eluent, and subsequent recrystallization from diethyl ether and pentane gave 8.62 g (71% yield) of the title compound. ¹H NMR (400 MHz, CDCl₃) δ ppm 7.72 (d, 1H) 7.69 (d, 1H) 7.09 (dd, 1H) 3.89 (s, 3H).

### h) 2-Benzylsulfanyl-1-bromo-4-methoxy-benzene

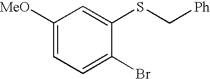

A mixture of 1-bromo-2-iodo-4-methoxy-benzene (12.43 g, 39.72 mmol), bis(dibenzylideneacetone)palladium (1.14 g, 1.99 mmol), 1,1′-bis(diphenylphosphino)ferrocene (1.11 g, 1.99 mmol) and triethylamine (11.2 mL, 79.44 mmol) in N,N-dimethylformamide (100 mL) was degassed with nitrogen for 15 minutes. Benzyl mercaptan (4.7 mL, 39.72 mmol) was added then and the reaction mixture was heated at 70° C. for 2.5 hours. The reaction mixture was diluted with ethyl acetate, washed with brine, dried over magnesium sulfate and concentrated under reduced pressure. Purification by column chromatography, using 4% diethyl ether in hexane as the eluent, gave 13.09 g (quantitative yield) of the title compound. ¹H NMR (400 MHz, CDCl₃) δ ppm 7.27-7.44 (m, 6H) 6.75 (d, 1H) 6.58 (dd, 1H) 4.14 (s, 2H) 3.70 (s, 3H).

### i) 1-Bromo-2-iodo-4-methoxy-benzene

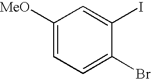

Bromine (2.8 mL, 54.49 mmol) was added dropwise over 5 minutes to a solution of 3-iodoanisole (10.01 g, 42.77 mmol) in glacial acetic acid (65 mL). The resulting orange solution was stirred at room temperature for 24 hours. The reaction mixture was diluted with water and extracted with hexane. The combined extracts were washed with aqueous sodium thiosulfate solution (5%) and brine, dried over magnesium sulfate and concentrated under reduced pressure to give 13.51 g (quantitative yield) of the title compound. ¹H NMR (400 MHz, CDCl₃) δ ppm 7.47 (d, 1H) 7.39 (d, 1H) 6.77 (dd, 1H) 3.77 (s, 3H).

### EXAMPLE 77 and 78

### 4-Hydroxy-benzene-1,2-disulfonic acid 2-amide 1-[4-(3,3-dimethyl-but-1-ynyl)-benzoylamide] (77) and 4-methoxy-benzene-1,2-disulfonic acid 2-amide 1-[4-(3,3-dimethyl-but-1-ynyl)-benzoylamide](78)

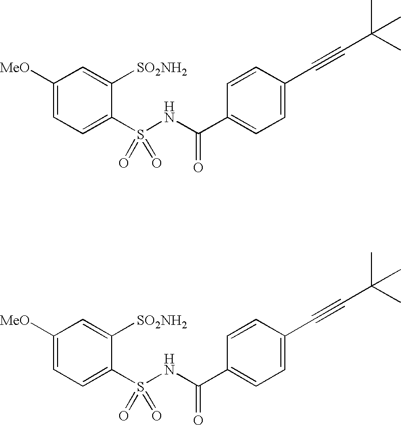

To a degassed mixture of 4-hydroxy-benzene-1,2-disulfonic acid 2-amide 1-(4-bromo-benzoylamide) and 4-methoxy-benzene-1,2-disulfonic acid 2-amide 1-(4-bromo-benzoylamide) (0.34 g, 0.71 mmol) and triethylamine (5.0 mL, 35.66 mmol) in anhydrous tetrahydrofuran (16 mL) were successively added tetrakis(triphenylphosphine)palladium (0.17 g, 0.14 mmol), copper(I) iodide (14 mg, 0.07 mmol) and 3,3-dimethyl-1-butyne (0.35 mL, 2.85 mmol) at room temperature. The reaction mixture was heated in as sealed tube at 70° C. overnight. The reaction mixture was cooled to room temperature, diluted with ethyl acetate and methanol. The organic phase was washed with hydrochloric acid (1 M, 15 mL) and brine, and concentrated under reduced pressure. Purification by preparative HPLC gave 16 mg of 4-hydroxy-benzene-1,2-disulfonic acid 2-amide 1-[4-(3,3-dimethyl-but-1-ynyl)-benzoylamide] and 25 mg of 4-methoxy-benzene-1,2-disulfonic acid 2-amide 1-[4-(3,3-dimethyl-but-1-ynyl)-benzoylamide].

4-Hydroxy-benzene-1,2-disulfonic acid 2-amide 1-[4-(3,3-dimethyl-but-1-ynyl)-benzoylamide] (77): ¹H NMR (400 MHz, CD₃OD) ppm δ 8.13 (d, 1H) 7.89 (d, 2H) 7.57 (d, 1H) 7.30 (d, 2H) 6.98 (dd, 1H) 1.31 (s, 9H); MS (ESI) m/z 434.9 [M−1]⁻.

4-Methoxy-benzene-1,2-disulfonic acid 2-amide 1-[4-(3,3-dimethyl-but-1-ynyl)-benzoylamide] (78): ¹H NMR (400 MHz, CD₃OD) ppm δ 8.41 (d, 1H) 7.79 (d, 2H) 7.72 (d, 1H) 7.40 (d, 2H) 7.31 (dd, 1H) 3.95 (s, 3H) 1.31 (s, 9H); MS (ESI) m/z 448.9 [M−1]⁻.

### a) 4-Hydroxy-benzene-1,2-disulfonic acid 2-amide 1-(4-bromo-benzoylamide) and 4-methoxy-benzene-1,2-disulfonic acid 2-amide 1-(4-bromo-benzoylamide)

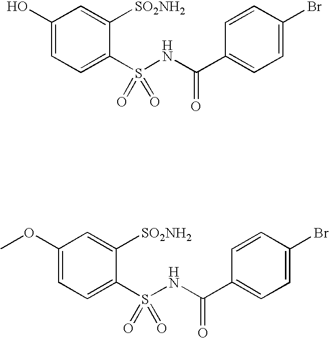

Boron tribromide (1 M in dichloromethane, 5.6 mL, 5.54 mmol) was added dropwise to a suspension of 4-methoxy-benzene-1,2-disulfonic acid 1-(4-bromo-benzoylamide) 2-tert-butylamide (0.40 g, 0.79 mmol) in dichloromethane (24 mL) at −20° C. The reaction mixture was allowed to warm to room temperature and stirred overnight. The reaction mixture was cooled to −20° C., methanol (10 mL) was added slowly and the volatiles were removed under reduced pressure. The residue was taken up in methanol (10 mL), the mixture was concentrated in vacuo and the process was repeated once more. Boron tribromide (1 M in dichloromethane, 5.6 mL, 5.54 mmol) was added and the mixture was stirred at room temperature for 18 hours. Additional boron tribromide (1 M in dichloromethane, 16 mL, 15.8 mmol) was added and the mixture was stirred at room temperature for 18 hours to give 354 mg of a mixture of 4-hydroxy-benzene-1,2-disulfonic acid 2-amide 1-(4-bromo-benzoylamide) and 4-methoxy-benzene-1,2-disulfonic acid 2-amide 1-(4-bromo-benzoylamide) (ratio 1.24:1 based on ¹H NMR) which was used directly in the next step without further purification. MS (ESI) m/z 433.2, 435.1 [M−1]⁻ and MS (ESI) m/z 447.2, 449.2 [M−1]⁻.

### EXAMPLE 79

### 3-Methoxy-N-(2-sulfamoylphenylsulfonyl)-4-(p-tolylethynyl)benzamide

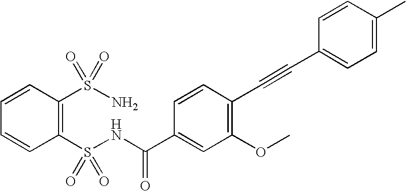

N,N′-Carbonyldiimidazole (33.1 g, 204.20 mmol) was added to a solution of 3-methoxy-4-(p-tolylethynyl)benzoic acid (47.7 g, 170.17 mmol) in N,N-dimethylformamide (400 mL) at room temperature under an atmosphere of argon. The mixture was stirred at 75° C. for 1.5 h and allowed to reach room temperature before benzene-1,2-disulfonamide (40.7 g, 165.07 mmol) and 4-(dimethylamino)pyridine (4.16 g, 34.03 mmol) were added. The reaction mixture was stirred at 70° C. overnight. The heating was turned off after 24 h but the reaction was left at room temperature overnight. The mixture was concentrated and ethyl acetate and water was added followed by dropwise addition over 40 min of hydrochloric acid (2 M, 150 mL). A gum-solid was formed. The solvent was decanted and methanol was added. The formed solid was removed by filtration, toluene (3 L) was added and the formed slurry was stirred at 110° C. for 40 h. The mixture was cooled to 50° C., filtered and dried in a vacuum cabinet for 6 h at 45° C. to give 34.8 g (42% yield) of the title compound. ¹H NMR (600 MHz, DMSO-d₆) δ ppm 8.37 (dd, 1H) 8.17 (dd, 1H), 7.87-7.98 (m, 2H), 7.59 (d, 1H), 7.56 (d, 1H), 7.49 (dd, 1H), 7.40-7.47 (m, 4H), 7.25 (d, 2H), 3.91 (s, 3H), 2.34 (s, 3H); MS (APCI) m/z 483 [M−1]⁻; MS (APCI) m/z 485 [M+1]⁺.

3-Methoxy-N-(2-sulfamoylphenylsulfonyl)-4-(p-tolylethynyl)benzamide form crystalline sodium- and potassium salts and crystalline salts from the following amines: piperazine, tert-butylamine, diethylamine and diethanolamine

### a) 3-Methoxy-4-(p-tolylethynyl)benzoic acid

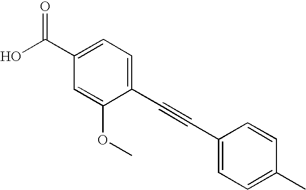

Aqueous sodium hydroxide (2 M, 400 mL, 800 mmol) was added to methyl 3-methoxy-4-(p-tolylethynyl)benzoate (81 g, 288.96 mmol) in tetrahydrofuran (500 mL) in a 2-L rector. The mixture was heated at 50° C. overnight and the mixture was concentrated until almost all tetrahydrofuran was evaporated. Ethyl acetate and water was added. The organic phase was concentrated and acetonitrile was added The formed solid was removed by filtration and was partitioned between ethyl acetate, water and acidified to pH˜2 with hydrochloric acid (6 M). The organic phase was concentrated, toluene and ethyl acetate was added and the solid was removed by filtration to give 49 g (64% yield) of the title compound. 1H NMR (400 MHz, DMSO-d₆) δ ppm 13.19 (br. s., 1H) 7.57-7.61 (m, 1H), 7.52-7.57 (m, 2H), 7.44 (m, 2H), 7.25 (m, 2H), 3.92 (s, 3H), 2.34 (s, 3H).

### b) Methyl 3-methoxy-4-(p-tolylethynyl)benzoate

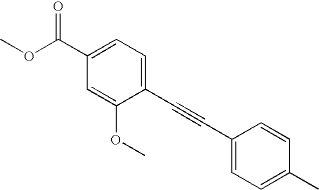

Methyl 4-bromo-3-methoxybenzoate (81 g, 330.52 mmol), 2-dicyclohexylphosphino-2′,4′,6′-triisopropylbiphenyl (9.45 g, 19.83 mmol), trans-bis(acetonitrile)palladium(II) chloride (1.715 g, 6.61 mmol) and cesium carbonate (258 g, 793.24 mmol) in acetonitrile (850 mL) were added to a 2-L reactor. The mixture was degassed, put under an atmosphere of nitrogen before p-tolylacetylene (41.9 mL, 330.52 mmol) was added and the mixture was heated at 70° C. overnight. p-Tolylacetylene (5 mL) was added and the mixture was stirred for an additional 7 hours. The mixture was allowed to reach room temperature before it was filtrated and concentrated. The residue was partitioned between ethyl acetate (1.3 L) and water (600 mL). Charcoal was added to the organic phase, the mixture was filtrated trough celite and concentrated to give 87.1 g (94% yield) of the title compound. ¹H NMR (600 MHz, CDCl₃) δ ppm 7.63 (dd, 1H) 7.57 (d, 1H), 7.54 (d, 1H), 7.47 (d, 2 H), 7.17 (d, 2H), 3.98 (s, 3H), 3.94 (s, 3H), 2.38 (s, 3H).

### Assays for Determining Biological Activity

### Inhibition of Prostaglandin E Synthase Activity

Compounds were tested as inhibitors of microsomal prostaglandin E synthase activity in microsomal prostaglandin E synthase assays and whole cell assays. These assays measure prostaglandin E2 (PGE2) synthesis which is taken as a measure of prostaglandin E synthase activity. Microsomal prostaglandin E synthase biochemical assays used microsomal prostaglandin E synthase-1 in microsomal preparations. The source of the microsomes can be for example interleukin-1β-stimulated human A549 cells (which express human mPGES-1) or Sf9 cells transfected with plasmids encoding human mPGES-1 cDNA.

The whole blood assay [described by Patrignani, P. et al, Journal of Pharmacology and Experimental Therapeutics, 1994, vol. 271, pp 1705-1712] was used as the whole cell assay for testing the compounds. Whole blood provides a protein and cell rich milieu for the study of biochemical efficacy of anti-inflammatory compounds such as prostaglandin synthase inhibitors. To study the inhibitory activities of these compounds, human blood was stimulated with lipopolysaccharide (LPS) for typically 16 hours to induce mPGES-1 expression, after which the concentration of produced PGE2 was measured by competitive-immuno assay (homogeneous time-resolved fluorescence, HTRF) as read out for effectiveness against mPGES-1-dependent PGE2 production.

### Microsomal Prostaglandin E Synthase Biochemical Assay

A solution of test compound was added to a diluted microsome preparation containing is human mPGES-1 and pre-incubated for 15 minutes in potassium phosphate buffer pH 6.8 with cofactor glutathione (GSH). Corresponding solutions without test compound were used as positive controls, and corresponding solutions without test compound and without microsomes were used as negative controls. The enzymatic reaction was then started by addition of the substrate PGH2 in an organic solution (dry acetonitrile).

The typical reaction conditions of the enzymatic reaction were thus: Test compound: ranging from 60 μM to 0.002 μM, or zero in positive and negative controls; potassium phosphate buffer pH 6.8:50 mM; GSH: 2.5 mM; mPGES-1-containing microsomes: 2 μg/mL (sample and positive controls) or 0 μg/mL (negative control); PGH2:10.8 μM; Acetonitrile: 7.7% (v/v); DMSO: 0.6% (v/v). The reaction was stopped after one minute by adding an acidic solution (pH 1.9) of ferric chloride and citrate (final concentrations 7 mM and 47 mM respectively), by which the PGH2 was sequestered (the PGH2 is reduced to mainly 12-hydroxy heptadecatrineoic acid (12-HHT) which is not detected by the subsequent PGE2 detection step). The resulting solution was then pH neutralized by addition of potassium phosphate buffer, prior to diluting an aliquot of the resulting solution in a weak potassium phosphate buffer (50 mM, pH 6.8) containing 0.2% BSA (w/v). [Adapted from Jacobsson et al., Proc. Natl. Acad. Sci. USA, 1999, vol. 96, pp. 7220-7225] The PGE2 formed was quantified by use of a commercial HTRF based kit (catalogue #62PG2PEC or #62P2APEC from Cisbio International). 100% activity was defined as the PGE2 production in positive controls subtracted by the PGE2 production in the negative controls. IC50 values were then determined using standard procedures.

Data from this assay for representative compounds is shown in the Table below. The potency is expressed as IC50 and the value indicated is an average of at least n=2. The data indicate that the compounds of the invention are expected to possess useful therapeutic properties.

| Example No. | IC₅₀ (μM) |
|---|---|
| 1 | 0.28 |
| 2 | 0.031 |
| 3 | 0.0082 |
| 4 | 0.005 |
| 5 | 0.014 |
| 6 | 0.025 |
| 7 | 0.0045 |
| 8 | 0.0064 |
| 9 | 0.004 |
| 10 | 0.0037 |
| 11 | 0.01 |
| 12 | 0.0044 |
| 13 | 0.0057 |
| 14 | 0.011 |
| 15 | 0.0084 |
| 16 | 0.016 |
| 17 | 0.0063 |
| 18 | 0.012 |
| 19 | 0.0073 |
| 20 | 0.012 |
| 21 | 0.005 |
| 22 | 0.013 |
| 23 | 0.034 |
| 24 | 2.9 |
| 25 | 0.13 |
| 26 | 0.085 |
| 27 | 0.085 |
| 28 | 0.02 |
| 29 | 0.39 |
| 30 | 0.13 |
| 31 | 0.22 |
| 32 | 0.06 |
| 33 | 0.054 |
| 34 | 0.012 |
| 35 | 0.014 |
| 36 | 0.018 |
| 37 | 0.035 |
| 38 | 0.0073 |
| 39 | 0.022 |
| 40 | 0.014 |
| 41 | 0.0057 |
| 42 | 0.037 |
| 43 | 0.0065 |
| 44 | 0.059 |
| 45 | 0.018 |
| 46 | 0.2 |
| 47 | 0.0065 |
| 48 | 0.01 |
| 49 | 0.013 |
| 50 | 0.017 |
| 51 | 0.026 |
| 52 | 0.05 |
| 53 | 0.025 |
| 54 | 0.028 |
| 55 | 0.08 |
| 56 | 0.1 |
| 57 | 0.0085 |
| 58 | 0.024 |
| 59 | 0.0085 |
| 60 | 0.03 |
| 61 | 0.027 |
| 62 | 0.0091 |
| 63 | 0.0093 |
| 64 | 0.015 |
| 65 | 0.0062 |
| 66 | 0.0094 |
| 67 | 0.0096 |
| 68 | 0.012 |
| 69 | 0.011 |
| 70 | 0.0078 |
| 71 | 0.031 |
| 72 | 0.043 |
| 73 | 0.024 |
| 74 | 0.19 |
| 75 | 0.026 |
| 76 | 0.53 |
| 77 | 0.061 |
| 78 | 2.2 |
| 79 | 0.0063 |

### Whole Blood Assay

Human blood collected from human volunteers in heparinized tubes was incubated with 100 μM acetyl salicylic acid, in order to inhibit the constitutively expressed cyclooxygenase (COX)-1/COX-2 enzymes, and then stimulated with 0.1 μg/ml LPS to induce the expression of enzymes along the COX-2 pathway, e.g. COX-2 and mPGES-1. 100 μL of this blood was added to the wells of a 384-well plate containing 1 μL DMSO solutions of compounds typically in the final concentration range 316 μM to 0.01 μM. Naproxen was used as reference compound. The mix was incubated at 37° C. for 16 hours. Plasma was harvested by centrifugation and stored at −70° C. until further analysis of PGE2 levels. For the calculations, the 0%-activity value was represented by blood treated with acetyl salicylic acid, LPS and the reference compound (1 mM Naproxen). The 100%-activity value was represented by blood treated with aspirin, LPS and DMSO. [Reference: Patrignani, P. et al, Journal of Pharmacology and Experimental Therapeutics, 1994, vol. 271, pp 1705-1712]. The PGE2 formed was quantified, after dilution in a weak potassium phosphate buffer (50 mM, pH 6.8) containing 0.2% BSA (w/v), by use of a commercial HTRF based kit (catalogue #62PG2PEC or #62P2APEC from Cisbio International). IC50 values were then determined using standard procedures.

1-15. (canceled)

16. A compound of formula (I) or a pharmaceutically acceptable salt thereof 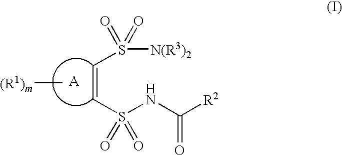
wherein:
A is selected from mono- and bicyclic aryl, mono- and bicyclic heteroaryl, cycloalkenyl and mono- and bicyclic heterocyclyl;
R¹ is independently selected from halogen, nitro, SF₅, CHO, C₀₋₆alkylCN, OC₁₋₆alkylCN, C₀₋₆alkylOR⁵, OC₂₋₆alkylOR⁵, C₀₋₆alkylNR⁵R⁶, OC₂₋₆alkylNR⁵R⁶, OC₂₋₆alkylOC₂₋₆alkylNR⁵R⁶, C₀₋₆alkylCO₂R⁵, OC₁₋₆alkylCO₂R⁵, C₀₋₆alkylCON(R⁵)₂, OC₁₋₆alkylCON(R⁵)₂, OC₂₋₆alkylNR⁵(CO)R⁶, C₀₋₆alkylNR⁵(CO)R⁶, O(CO)NR⁵R⁶, NR⁵(CO)OR⁶, NR⁵(CO)NR⁵R⁶, O(CO)OR⁵, O(CO)R⁵, C₀₋₆alkylCOR⁵, OC₁₋₆alkylCOR⁵, NR⁵(CO)(CO)R⁵, NR⁵(CO)(CO)NR⁵R⁶, C₀₋₆alkylSR⁵, C₀₋₆alkyl(SO₂)NR⁵R⁶, OC₁₋₆alkylNR⁵(SO₂)R⁶, OC₀₋₆alkyl(SO₂)NR⁵R⁶, C₀₋₆alkyl(SO)NR⁵R⁶, OC₁₋₆alkyl(SO)NR⁵R⁶, C₀₋₆alkylOSO₂R⁵, C₀₋₆alkylNR⁵(SO₂)NR⁵R⁶, C₀₋₆alkylNR⁵(SO)R⁶, OC₂₋₆alkylNR⁵(SO)R⁶, OC₁₋₆alkylSO₂R⁵, C₀₋₆alkylSO₂R⁵, C₀₋₆alkylSOR⁵, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl or C₀₋₆alkylheterocyclyl is optionally substituted with one or more B, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with one or more B;
R² is -L¹-G¹-L²-G²;
R³ is hydrogen;
G¹ is selected from C₃₋₁₀cycloalkyl, C₄₋₁₂cycloalkenyl, C₇₋₁₂cycloalkynyl, aryl, heteroaryl, heterocyclyl, wherein said C₃₋₁₀cycloalkyl, C₄₋₁₂cycloalkenyl, C₇₋₁₂cycloalkynyl, aryl, heteroaryl or heterocyclyl is optionally substituted with one or more R¹⁰;
G² is selected from hydrogen, C₃₋₈cycloalkyl, C₄₋₁₂cycloalkenyl, C₇₋₁₂cycloalkynyl, aryl, heteroaryl, heterocyclyl, wherein said C₃₋₈cycloalkyl, C₄₋₁₂cycloalkenyl, C₇₋₁₂cycloalkynyl, aryl, heteroaryl or heterocyclyl is optionally substituted with one or more R¹⁰;
At each occurrence, R⁵ is independently selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl or C₀₋₆alkylheterocyclyl is optionally substituted with one or more B;
At each occurrence, R⁶ is selected from hydrogen, C₁₋₆alkyl, C₂₋₆alkenyl, C₂₋₆alkynyl, C₀₋₆alkylOR⁵, C₀₋₆alkylC₃₋₈cycloalkyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl or C₀₋₆alkylheterocyclyl is optionally substituted with one or more B; or
R⁵ and R⁶ may together with the linking atom or atoms to which they are bonded form a 4 to 6 membered heterocyclic ring containing one or more heteroatoms selected from N, O or S that is optionally substituted with B; whenever two R⁵ groups occur in the structure then they may optionally together with the linking atom or atoms to which they are bonded form a 5 or 6 membered heterocyclic ring containing one or more heteroatoms selected from N, O or S, that is optionally substituted with one or more B
L¹ and L² independently represent a bond or a 1-7 membered non-cyclic linking group containing 0-2 heteroatoms selected from O, N, and S, said linking group optionally containing CO, S(O)ₙ, C═C or an acetylenic group, and optionally being substituted with one or more R⁸;
R⁸ is selected from halogen, nitro, CHO, CN, OH, OC₁₋₆alkyl, O(C₁₋₆alkyl)O(C₁₋₆alkyl), C₁₋₆alkyl, C₂₋₆alkenyl, C₂₋₆alkynyl N(C₁₋₆alkyl)(C₁₋₆alkyl), NH₂, NH(C₁₋₆alkyl), S(O)ₙ(C₁₋₆alkyl), SO₂N(C₁₋₆alkyl)(C₁₋₆alkyl), SO₂NH₂, SO₂NH(C₁₋₆alkyl), CF₃, CHF₂, CFH₂, C(O)(C₁₋₆alkyl), C(O)N(C₁₋₆alkyl)(C₁₋₆alkyl), C(O)NH(C₁₋₆alkyl), C(O)NH₂, N(C₁₋₆alkyl)(CO)N(C₁₋₆alkyl)(C₁₋₆alkyl), NH(CO)N(C₁₋₆alkyl)(C₁₋₆alkyl), N(C₁₋₆alkyl)(CO)NH(C₁₋₆alkyl), NH(CO)NH₂, N(C₁₋₆alkyl)(CO)NH₂,
Whenever two R⁸ groups are connected to the same atom of the linking group L¹, they may optionally together form a 3 to 6 membered non-aromatic, carbocyclic or heterocyclic (containing one or more heteroatoms selected from N, O or S) ring, that is optionally substituted with one or more R⁹;
R⁹ is selected from halogen, nitro, CHO, CN, OH, OC₁₋₆alkyl, O(C₁₋₆alkyl)O(C₁₋₆alkyl), C₁₋₆alkyl, C₂₋₆alkenyl, C₂₋₆alkynyl N(C₁₋₆alkyl)(C₁₋₆alkyl), NH₂, NH(C₁₋₆alkyl), S(O)ₙ(C₁₋₆alkyl), SO₂N(C₁₋₆alkyl)(C₁₋₆alkyl), SO₂NH₂, SO₂NH(C₁₋₆alkyl), CF₃, CHF₂, CFH₂, C(O)(C₁₋₆alkyl), C(O)N(C₁₋₆alkyl)(C₁₋₆alkyl), C(O)NH(C₁₋₆alkyl), C(O)NH₂, N(C₁₋₆alkyl)(CO)N(C₁₋₆alkyl)(C₁₋₆alkyl), NH(CO)N(C₁₋₆alkyl)(C₁₋₆alkyl), N(C₁₋₆alkyl)(CO)NH(C₁₋₆alkyl), NH(CO)NH₂, N(C₁₋₆alkyl)(CO)NH₂;
B is selected from halogen, nitro, SF₅, OSF₅, CN, OR¹⁵, OC₂₋₆alkylNR¹⁵R¹⁶, NR¹⁵R¹⁶, CONR¹⁵R¹⁶, NR¹⁵(CO)R¹⁶, O(CO)C₁₋₆alkyl, (CO)OC₁₋₆alkyl, COR¹⁵, (SO₂)NR¹⁵R¹⁶, NR¹⁵SO₂R¹⁵, SO₂R¹⁵, SOR¹⁵, (CO)C₁₋₆alkylNR¹⁵R¹⁶, (SO₂)C₁₋₆alkylNR¹⁵R¹⁶, OSO₂R¹⁵, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
R¹⁵ is selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
R¹⁶ is selected from hydrogen, C₁₋₆alkyl, C₂₋₆alkenyl, C₂₋₆alkynyl, C₀₋₆alkylOR⁵, C₀₋₆alkylC₃₋₈cycloalkyl, C₀₋₆alkylaryl, C₀₋₆alkylheteroaryl and C₀₋₆alkylheterocyclyl; or
R¹⁵ and R¹⁶ may together with the linking atom or atoms to which they are bonded form a 4 to 6 membered heterocyclic ring containing one or more heteroatoms selected from N, O or S;
whenever two R¹⁵ groups occur in the structure then they may optionally together with the linking atom or atoms to which they are bonded form a 5 or 6 membered heterocyclic ring containing one or more heteroatoms selected from N, O or S;
D is selected from halogen, nitro, SF₅, OSF₅, CN, OR¹³, OC₂₋₆alkylNR¹³R¹⁴, NR¹³R¹⁴, CONR¹³R¹⁴, NR¹³(CO)R¹⁴, O(CO)C₁₋₆alkyl, (CO)OC₁₋₆alkyl, COR¹³, (SO₂)NR¹³R¹⁴, NR¹³SO₂R¹⁴, SO₂R¹³, SOR¹³, (CO)C₁₋₆alkylNR¹³R¹⁴, (SO₂)C₁₋₆alkylNR¹³R¹⁴, OSO₂R¹³, C₁₋₆alkyl, C₂₋₆alkenyl, C₂₋₆alkynyl, C₀₋₆alkylC₃₋₈cycloalkyl, and C₀₋₆alkylheterocyclyl;
R¹⁰ is independently selected from halogen, nitro, SF₅, OSF₅, CN, OR¹¹, C≡CR¹¹, OC₂₋₆alkylNR¹¹R¹², NR¹¹R¹², CONR¹¹R¹², NR¹¹(CO)R¹², O(CO)C₁₋₆alkyl, (CO)OC₁₋₆alkyl, COR¹¹, (SO₂)NR¹¹R¹², NR¹¹SO₂R¹¹, SO₂R¹¹, SOR¹¹, (CO)C₁₋₆alkylNR¹¹R¹², (SO₂)C₁₋₆alkylNR¹¹R¹², OSO₂R¹¹, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl, C₀₋₆alkylheterocyclyl and OC₂₋₆alkylheterocyclyl, wherein said C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl, C₀₋₆alkylheterocyclyl or OC₂₋₆alkylheterocyclyl is optionally substituted with one or more E, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with one or more E;
At each occurrence, R¹¹ is independently selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein any of the individual C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl groups may be optionally substituted with one or more E;
R¹² is selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl, wherein any of the individual C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl groups may be optionally substituted with one or more E; or
R¹¹ and R¹² may together with the linking atom or atoms to which they are bonded form a 4 to 6 membered heterocyclic ring containing one or more heteroatoms selected from N, O or S that is optionally substituted with B; whenever two R¹¹ groups occur in the structure then they may optionally together with the linking atom or atoms to which they are bonded form a 5 or 6 membered heterocyclic ring containing one or more heteroatoms selected from N, O or S, where the ring system is optionally substituted with one or more E;
R¹³ is independently selected from hydrogen, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
R¹⁴ is selected from hydrogen, C₁₋₆alkyl, C₂₋₆alkenyl, C₂₋₆alkynyl, C₀₋₆alkylOR⁵, C₀₋₆alkylC₃₋₈cycloalkyl, C₀₋₆alkylaryl, C₀₋₆alkylheteroaryl and C₀₋₆alkylheterocyclyl; or
R¹³ and R¹⁴ may together with the linking atom or atoms to which they are bonded form a 4 to 6 membered heterocyclic ring containing one or more heteroatoms selected from N, O or S; whenever two R¹³ groups occur in the structure then they may optionally together with the linking atom or atoms to which they are bonded form a 5 or 6 membered heterocyclic ring containing one or more heteroatoms selected from N, O or S;
E is selected from halogen, nitro, SF₅, OSF₅, CN, OR⁵, OC₂₋₆alkylNR⁶R⁶, NR⁶R⁶, CONR⁶R⁶, NR⁵(CO)R⁶, O(CO)C₁₋₆alkyl, (CO)OC₁₋₆alkyl, COR^{S}, (SO₂)NR⁵R⁶, NR⁵SO₂R⁵, SO₂R⁵, SOR⁵, (CO)C₁₋₆alkylNR⁵R⁶, (SO₂)C₁₋₆alkylNR⁶R⁶, OSO₂R⁵, C₁₋₆alkyl, C₂₋₆alkenyl, C₂₋₆alkynyl, C₀₋₆alkylC₃₋₈cycloalkyl, C₀₋₆alkylaryl, C₀₋₆alkylheteroaryl and C₀₋₆alkylheterocyclyl;
m=0, 1, 2, 3, 4;
n=0, 1, 2;
wherein said compound is selected from the group consisting of:
4-(Benzyloxy)-3-(cyclopropylmethoxy)-N-(2-sulfamoylphenylsulfonyl)benzamide;
3-Methoxy-N-(2-sulfamoylphenylsulfonyl)-4-(p-tolyloxymethyl)benzamide;
3-(2,2-Difluoroethoxy)-4-(phenylethynyl)-N-(2-sulfamoylphenylsulfonyl)benzamide;
3-(2,2-Difluoroethoxy)-N-(2-sulfamoylphenylsulfonyl)-4-(p-tolylethynyl)benzamide;
4-(Cyclopentylethynyl)-3-(2,2-difluoroethoxy)-N-(2-sulfamoylphenylsulfonyl)benzamide;
3-(2,2-Difluoroethoxy)-4-(3,3-dimethylbut-1-ynyl)-N-(2-sulfamoylphenylsulfonyl)-benzamide;
4-(Benzofuran-2-yl)-3-(2,2-difluoroethoxy)-N-(2-sulfamoylphenylsulfonyl)benzamide;
N-(2-Sulfamoylphenylsulfonyl)-4-(o-tolylethynyl)benzamide;
N-(2-sulfamoylphenylsulfonyl)-4-(p-tolylethynyl)benzamide;
4-((4-Chlorophenyl)ethynyl)-N-(2-sulfamoylphenylsulfonyl)benzamide;
4-((4-Fluorophenyl)ethynyl)-N-(2-sulfamoylphenylsulfonyl)benzamide;
4-((3-Chlorophenyl)ethynyl)-N-(2-sulfamoylphenylsulfonyl)benzamide;
4-((3-Fluorophenyl)ethynyl)-N-(2-sulfamoylphenylsulfonyl)benzamide;
3-Methoxy-N-(2-sulfamoylphenylsulfonyl)-4-(o-tolylethynyl)benzamide;
4-((4-Chlorophenyl)ethynyl)-3-methoxy-N-(2-sulfamoylphenylsulfonyl)benzamide;
4-((4-Fluorophenyl)ethynyl)-3-methoxy-N-(2-sulfamoylphenylsulfonyl)benzamide;
3-Methoxy-N-(2-sulfamoylphenylsulfonyl)-4-(p-tolylethynyl)benzamide;
3-Methoxy-N-(2-sulfamoylphenylsulfonyl)-4-(m-tolylethynyl)benzamide;
4-((3-Chlorophenyl)ethynyl)-3-methoxy-N-(2-sulfamoylphenylsulfonyl)benzamide;
4-((3-Fluorophenyl)ethynyl)-3-methoxy-N-(2-sulfamoylphenylsulfonyl)benzamide;
4-(Benzofuran-2-yl)-3-(cyclopropylmethoxy)-N-(2-sulfamoylphenylsulfonyl)benzamide;
3-(Cyclopropylmethoxy)-4-(3,3-dimethylbut-1-ynyl)-N-(2-sulfamoylphenylsulfonyl)-benzamide;
3-(Cyclopropylmethoxy)-4-(3-methylbut-1-ynyl)-N-(2-sulfamoylphenylsulfonyl)-benzamide;
N-(2-Sulfamoylphenylsulfonyl)-4-((3,3,4,4-tetrafluoropyrrolidin-1-yl)methyl)benzamide;
3-(3-Hydroxy-3-methylbutoxy)-4-(3-methylbut-1-ynyl)-N-(2-sulfamoylphenylsulfonyl)-benzamide;
3-Isopropoxy-4-(3-methylbut-1-ynyl)-N-(2-sulfamoylphenylsulfonyl)benzamide;
4-(Cyclopropylethynyl)-3-(3-methoxy-3-methylbutoxy)-N-(2-sulfamoylphenylsulfonyl)-benzamide;
3-Isobutoxy-4-(3-methylbut-1-ynyl)-N-(2-sulfamoylphenylsulfonyl)benzamide;
3-Methoxy-4-(4-methylbenzyloxy)-N-(2-sulfamoylphenylsulfonyl)benzamide;
4-(Benzyloxy)-N-(2-sulfamoylphenylsulfonyl)-3-(3,3,3-trifluoropropoxy)benzamide;
4-(3,3-Dicyclopropyl-3-hydroxyprop-1-ynyl)-N-(2-sulfamoylphenylsulfonyl)benzamide;
3-(Hydroxymethyl)-4-(3-methylhex-1-ynyl)-N-(2-sulfamoylphenylsulfonyl)benzamide;
3-Isopropoxy-N-(2-sulfamoylphenylsulfonyl)-4-((4-(trifluoromethyl)phenyl)ethynyl)-benzamide;
4-(3-Methylhex-1-ynyl)-N-(2-sulfamoylphenylsulfonyl)-3-(2-(2,2,2-trifluoroethoxy)-ethoxy)benzamide;
4-(3-Methylhex-1-ynyl)-N-(2-sulfamoylphenylsulfonyl)-3-(3,3,3-trifluoropropoxy)benzamide;
4-(3,3-Dicyclopropylprop-1-ynyl)-N-(2-sulfamoylphenylsulfonyl)-3-(3,3,3-trifluoropropoxy)benzamide;
3-Methoxy-4-(3-methylhex-1-ynyl)-N-(2-sulfamoylphenylsulfonyl)benzamide;
3-Methoxy-N-(2-sulfamoylphenylsulfonyl)-4-((4-(trifluoromethyl)phenyl)ethynyl)-benzamide;
3-(Hydroxymethyl)-N-(2-sulfamoylphenylsulfonyl)-4-((4-(trifluoromethyl)phenyl)-ethynyl)benzamide;
4-(3-Methylhex-1-ynyl)-N-(2-sulfamoylphenylsulfonyl)benzamide;
N-(2-Sulfamoylphenylsulfonyl)-4-((4-(trifluoromethyl)phenyl)ethynyl)benzamide;
4-(6-Chlorohex-1-ynyl)-N-(2-sulfamoylphenylsulfonyl)benzamide;
N-(2-Sulfamoylphenylsulfonyl)-6-((4-(trifluoromethyl)phenyl)ethynyl)nicotinamide;
5-Chloro-6-(3-methylbut-1-ynyl)-N-(2-sulfamoylphenylsulfonyl)nicotinamide;
4-(3-Methylbut-1-ynyl)-N-(2-sulfamoylphenylsulfonyl)-3-(3,3,3-trifluoropropoxy)-benzamide;
5-Fluoro-6-(3-methylbut-1-ynyl)-N-(2-sulfamoylphenylsulfonyl)nicotinamide;
5-Fluoro-N-(2-sulfamoylphenylsulfonyl)-6-((4-(trifluoromethyl)phenyl)ethynyl)-nicotinamide;
N-(2-Sulfamoylphenylsulfonyl)-6-((4-(trifluoromethyl)phenyl)ethynyl)-5-(3,3,3-trifluoro-propoxy)nicotinamide;
6-(3-Methylbut-1-ynyl)-N-(2-sulfamoylphenylsulfonyl)-5-(3,3,3-trifluoropropoxy)-nicotinamide;
3-Methyl-4-(3-methylbut-1-ynyl)-N-(2-sulfamoylphenylsulfonyl)benzamide;
4-(3-Methylbut-1-ynyl)-N-(2-sulfamoylphenylsulfonyl)-3-(2-(2,2,2-trifluoroethoxy)-ethoxy)benzamide;
3-(2,2-Difluoroethoxy)-4-(3-methylbut-1-ynyl)-N-(2-sulfamoylphenylsulfonyl)-benzamide;
4-(3-Methylbut-1-ynyl)-N-(2-sulfamoylphenylsulfonyl)-3-(2,2,2-trifluoroethoxy)-benzamide;
4-(3-Methylbut-1-ynyl)-N-(2-sulfamoylphenylsulfonyl)-3-(trifluoromethoxy)-benzamide;
4-(3-Methylbut-1-ynyl)-N-(2-sulfamoylphenylsulfonyl)-3-((3,3,3-trifluoropropoxy)-methyl)benzamide;
4-(3-Methylbut-1-ynyl)-N-(2-sulfamoylphenylsulfonyl)-3-((2,2,2-trifluoroethoxy)methyl)-benzamide;
4-(Benzofuran-2-yl)-3-fluoro-N-(2-sulfamoylphenylsulfonyl)benzamide;
4-(3-Methylbut-1-ynyl)-N-(2-sulfamoylphenylsulfonyl)-3-(2,2,3,3-tetrafluoropropoxy)-benzamide;
4-(Benzofuran-2-yl)-N-(2-sulfamoylphenylsulfonyl)-3-(3,3,3-trifluoropropoxy)benzamide;
4-(3,3-Dimethylbut-1-ynyl)-N-(2-sulfamoylphenylsulfonyl)-3-(3,3,3-trifluoropropoxy)-benzamide;
4-(3,3-Dimethylbut-1-ynyl)-N-(2-sulfamoylphenylsulfonyl)-3-(2-(2,2,2-trifluoroethoxy)-ethoxy)benzamide;
4-(Benzofuran-2-yl)-N-(2-sulfamoylphenylsulfonyl)-3-(2-(2,2,2-trifluoroethoxy)ethoxy)-benzamide;
4-(Phenylethynyl)-N-(2-sulfamoylphenylsulfonyl)-3-(2-(2,2,2-trifluoroethoxy)ethoxy)-benzamide;
4-(3-Methylbut-1-ynyl)-N-(2-sulfamoylphenylsulfonyl)-3-(4,4,4-trifluorobutoxy)-benzamide;
4-(Phenylethynyl)-N-(2-sulfamoylphenylsulfonyl)-3-(3,3,3-trifluoropropoxy)benzamide;
4-(Cyclopentylethynyl)-N-(2-sulfamoylphenylsulfonyl)-3-(3,3,3-trifluoropropoxy)-benzamide;
4-(Cyclopentylethynyl)-N-(2-sulfamoylphenylsulfonyl)-3-(2-(2,2,2-trifluoroethoxy)-ethoxy)benzamide;
4-(Cyclopentylethynyl)-3-(3,3-difluoropropoxy)-N-(2-sulfamoylphenylsulfonyl)-benzamide;
4-(Cyclopentylethynyl)-N-(2-sulfamoylphenylsulfonyl)-3-(4,4,4-trifluorobutoxy)-benzamide;
3-(3,3-Difluoropropoxy)-N-(2-sulfamoylphenylsulfonyl)-4-(p-tolylethynyl)benzamide;
3-(3,3-Difluoropropoxy)-4-(3-methylbut-1-ynyl)-N-(2-sulfamoylphenylsulfonyl)-benzamide;
3-(3-Fluoropropoxy)-4-(3-methylbut-1-ynyl)-N-(2-sulfamoylphenylsulfonyl)benzamide;
3-Methoxy-4-(phenylethynyl)-N-(2-sulfamoylphenylsulfonyl)benzamide;
3-Methoxy-4-(3-methylbut-1-ynyl)-N-(2-sulfamoylphenylsulfonyl)benzamide;
4-Hydroxy-benzene-1,2-disulfonic acid 2-amide 1-(4-benzofuran-2-yl-benzoylamide);
4-Methoxy-benzene-1,2-disulfonic acid 2-amide 1-(4-benzofuran-2-yl-benzoylamide);
4-Hydroxy-benzene-1,2-disulfonic acid 2-amide 1-[4-(3,3-dimethyl-but-1-ynyl)-benzoylamide];
4-Methoxy-benzene-1,2-disulfonic acid 2-amide 1-[4-(3,3-dimethyl-but-1-ynyl)-benzoylamide] and
3-Methoxy-N-(2-sulfamoylphenylsulfonyl)-4-(p-tolylethynyl)benzamide; or a pharmaceutically acceptable salt thereof.

17. A compound according to claim 16 selected from the group consisting of:
4-(Benzyloxy)-3-(cyclopropylmethoxy)-N-(2-sulfamoylphenylsulfonyl)benzamide;
3-Methoxy-N-(2-sulfamoylphenylsulfonyl)-4-(p-tolyloxymethyl)benzamide;
3-(2,2-Difluoroethoxy)-4-(phenylethynyl)-N-(2-sulfamoylphenylsulfonyl)benzamide;
3-(2,2-Difluoroethoxy)-N-(2-sulfamoylphenylsulfonyl)-4-(p-tolylethynyl)benzamide;
4-(Cyclopentylethynyl)-3-(2,2-difluoroethoxy)-N-(2-sulfamoylphenylsulfonyl)benzamide;
3-(2,2-Difluoroethoxy)-4-(3,3-dimethylbut-1-ynyl)-N-(2-sulfamoylphenylsulfonyl)-benzamide;
4-(Benzofuran-2-yl)-3-(2,2-difluoroethoxy)-N-(2-sulfamoylphenylsulfonyl)benzamide;
N-(2-Sulfamoylphenylsulfonyl)-4-(o-tolylethynyl)benzamide;
N-(2-sulfamoylphenylsulfonyl)-4-(p-tolylethynyl)benzamide;
4-((4-Chlorophenyl)ethynyl)-N-(2-sulfamoylphenylsulfonyl)benzamide;
4-((4-Fluorophenyl)ethynyl)-N-(2-sulfamoylphenylsulfonyl)benzamide;
4-((3-Chlorophenyl)ethynyl)-N-(2-sulfamoylphenylsulfonyl)benzamide;
4-((3-Fluorophenyl)ethynyl)-N-(2-sulfamoylphenylsulfonyl)benzamide;
3-Methoxy-N-(2-sulfamoylphenylsulfonyl)-4-(o-tolylethynyl)benzamide;
4-((4-Chlorophenyl)ethynyl)-3-methoxy-N-(2-sulfamoylphenylsulfonyl)benzamide;
4-((4-Fluorophenyl)ethynyl)-3-methoxy-N-(2-sulfamoylphenylsulfonyl)benzamide;
3-Methoxy-N-(2-sulfamoylphenylsulfonyl)-4-(p-tolylethynyl)benzamide;
3-Methoxy-N-(2-sulfamoylphenylsulfonyl)-4-(m-tolylethynyl)benzamide;
4-((3-Chlorophenyl)ethynyl)-3-methoxy-N-(2-sulfamoylphenylsulfonyl)benzamide;
4-((3-Fluorophenyl)ethynyl)-3-methoxy-N-(2-sulfamoylphenylsulfonyl)benzamide;
4-(Benzofuran-2-yl)-3-(cyclopropylmethoxy)-N-(2-sulfamoylphenylsulfonyl)benzamide;
3-(Cyclopropylmethoxy)-4-(3,3-dimethylbut-1-ynyl)-N-(2-sulfamoylphenylsulfonyl)-benzamide;
3-(Cyclopropylmethoxy)-4-(3-methylbut-1-ynyl)-N-(2-sulfamoylphenylsulfonyl)-benzamide;
N-(2-Sulfamoylphenylsulfonyl)-4-((3,3,4,4-tetrafluoropyrrolidin-1-yl)methyl)benzamide;
3-(3-Hydroxy-3-methylbutoxy)-4-(3-methylbut-1-ynyl)-N-(2-sulfamoylphenylsulfonyl)-benzamide;
3-Isopropoxy-4-(3-methylbut-1-ynyl)-N-(2-sulfamoylphenylsulfonyl)benzamide;
4-(Cyclopropylethynyl)-3-(3-methoxy-3-methylbutoxy)-N-(2-sulfamoylphenylsulfonyl)-benzamide;
3-Isobutoxy-4-(3-methylbut-1-ynyl)-N-(2-sulfamoylphenylsulfonyl)benzamide;
3-Methoxy-4-(4-methylbenzyloxy)-N-(2-sulfamoylphenylsulfonyl)benzamide;
4-(Benzyloxy)-N-(2-sulfamoylphenylsulfonyl)-3-(3,3,3-trifluoropropoxy)benzamide;
4-(3,3-Dicyclopropyl-3-hydroxyprop-1-ynyl)-N-(2-sulfamoylphenylsulfonyl)-benzamide;
3-(Hydroxymethyl)-4-(3-methylhex-1-ynyl)-N-(2-sulfamoylphenylsulfonyl)benzamide;
3-Isopropoxy-N-(2-sulfamoylphenylsulfonyl)-4-((4-(trifluoromethyl)phenyl)ethynyl)-benzamide;
4-(3-Methylhex-1-ynyl)-N-(2-sulfamoylphenylsulfonyl)-3-(2-(2,2,2-trifluoroethoxy)-ethoxy)benzamide;
4-(3-Methylhex-1-ynyl)-N-(2-sulfamoylphenylsulfonyl)-3-(3,3,3-trifluoropropoxy)benzamide;
4-(3,3-Dicyclopropylprop-1-ynyl)-N-(2-sulfamoylphenylsulfonyl)-3-(3,3,3-trifluoropropoxy)benzamide;
3-Methoxy-4-(3-methylhex-1-ynyl)-N-(2-sulfamoylphenylsulfonyl)benzamide;
3-Methoxy-N-(2-sulfamoylphenylsulfonyl)-4-((4-(trifluoromethyl)phenyl)ethynyl)-benzamide;
3-(Hydroxymethyl)-N-(2-sulfamoylphenylsulfonyl)-4-((4-(trifluoromethyl)phenyl)-ethynyl)benzamide;
4-(3-Methylhex-1-ynyl)-N-(2-sulfamoylphenylsulfonyl)benzamide;
N-(2-Sulfamoylphenylsulfonyl)-4-((4-(trifluoromethyl)phenyl)ethynyl)benzamide;
4-(6-Chlorohex-1-ynyl)-N-(2-sulfamoylphenylsulfonyl)benzamide;
N-(2-Sulfamoylphenylsulfonyl)-6-((4-(trifluoromethyl)phenyl)ethynyl)nicotinamide;
5-Chloro-6-(3-methylbut-1-ynyl)-N-(2-sulfamoylphenylsulfonyl)nicotinamide;
4-(3-Methylbut-1-ynyl)-N-(2-sulfamoylphenylsulfonyl)-3-(3,3,3-trifluoropropoxy)-benzamide;
5-Fluoro-6-(3-methylbut-1-ynyl)-N-(2-sulfamoylphenylsulfonyl)nicotinamide;
5-Fluoro-N-(2-sulfamoylphenylsulfonyl)-6-((4-(trifluoromethyl)phenyl)ethynyl)-nicotinamide;
N-(2-Sulfamoylphenylsulfonyl)-6-((4-(trifluoromethyl)phenyl)ethynyl)-5-(3,3,3-trifluoro-propoxy)nicotinamide;
6-(3-Methylbut-1-ynyl)-N-(2-sulfamoylphenylsulfonyl)-5-(3,3,3-trifluoropropoxy)-nicotinamide;
3-Methyl-4-(3-methylbut-1-ynyl)-N-(2-sulfamoylphenylsulfonyl)benzamide;
4-(3-Methylbut-1-ynyl)-N-(2-sulfamoylphenylsulfonyl)-3-(2-(2,2,2-trifluoroethoxy)-ethoxy)benzamide;
3-(2,2-Difluoroethoxy)-4-(3-methylbut-1-ynyl)-N-(2-sulfamoylphenylsulfonyl)-benzamide;
4-(3-Methylbut-1-ynyl)-N-(2-sulfamoylphenylsulfonyl)-3-(2,2,2-trifluoroethoxy)-benzamide;
4-(3-Methylbut-1-ynyl)-N-(2-sulfamoylphenylsulfonyl)-3-(trifluoromethoxy)-benzamide;
4-(3-Methylbut-1-ynyl)-N-(2-sulfamoylphenylsulfonyl)-3-((3,3,3-trifluoropropoxy)-methyl)benzamide;
4-(3-Methylbut-1-ynyl)-N-(2-sulfamoylphenylsulfonyl)-3-((2,2,2-trifluoroethoxy)methyl)-benzamide;
4-(Benzofuran-2-yl)-3-fluoro-N-(2-sulfamoylphenylsulfonyl)benzamide;
4-(3-Methylbut-1-ynyl)-N-(2-sulfamoylphenylsulfonyl)-3-(2,2,3,3-tetrafluoropropoxy)-benzamide;
4-(Benzofuran-2-yl)-N-(2-sulfamoylphenylsulfonyl)-3-(3,3,3-trifluoropropoxy)benzamide;
4-(3,3-Dimethylbut-1-ynyl)-N-(2-sulfamoylphenylsulfonyl)-3-(3,3,3-trifluoropropoxy)-benzamide;
4-(3,3-Dimethylbut-1-ynyl)-N-(2-sulfamoylphenylsulfonyl)-3-(2-(2,2,2-trifluoroethoxy)-ethoxy)benzamide;
4-(Benzofuran-2-yl)-N-(2-sulfamoylphenylsulfonyl)-3-(2-(2,2,2-trifluoroethoxy)ethoxy)-benzamide;
4-(Phenylethynyl)-N-(2-sulfamoylphenylsulfonyl)-3-(2-(2,2,2-trifluoroethoxy)ethoxy)-benzamide;
4-(3-Methylbut-1-ynyl)-N-(2-sulfamoylphenylsulfonyl)-3-(4,4,4-trifluorobutoxy)-benzamide;
4-(Phenylethynyl)-N-(2-sulfamoylphenylsulfonyl)-3-(3,3,3-trifluoropropoxy)benzamide;
4-(Cyclopentylethynyl)-N-(2-sulfamoylphenylsulfonyl)-3-(3,3,3-trifluoropropoxy)-benzamide;
4-(Cyclopentylethynyl)-N-(2-sulfamoylphenylsulfonyl)-3-(2-(2,2,2-trifluoroethoxy)-ethoxy)benzamide;
4-(Cyclopentylethynyl)-3-(3,3-difluoropropoxy)-N-(2-sulfamoylphenylsulfonyl)-benzamide;
4-(Cyclopentylethynyl)-N-(2-sulfamoylphenylsulfonyl)-3-(4,4,4-trifluorobutoxy)-benzamide;
3-(3,3-Difluoropropoxy)-N-(2-sulfamoylphenylsulfonyl)-4-(p-tolylethynyl)benzamide;
3-(3,3-Difluoropropoxy)-4-(3-methylbut-1-ynyl)-N-(2-sulfamoylphenylsulfonyl)-benzamide;
3-(3-Fluoropropoxy)-4-(3-methylbut-1-ynyl)-N-(2-sulfamoylphenylsulfonyl)benzamide;
3-Methoxy-4-(phenylethynyl)-N-(2-sulfamoylphenylsulfonyl)benzamide;
3-Methoxy-4-(3-methylbut-1-ynyl)-N-(2-sulfamoylphenylsulfonyl)benzamide;
4-Hydroxy-benzene-1,2-disulfonic acid 2-amide 1-(4-benzofuran-2-yl-benzoylamide);
4-Methoxy-benzene-1,2-disulfonic acid 2-amide 1-(4-benzofuran-2-yl-benzoylamide);
4-Hydroxy-benzene-1,2-disulfonic acid 2-amide 1-[4-(3,3-dimethyl-but-1-ynyl)-benzoylamide];
4-Methoxy-benzene-1,2-disulfonic acid 2-amide 1-[4-(3,3-dimethyl-but-1-ynyl)-benzoylamide] and
3-Methoxy-N-(2-sulfamoylphenylsulfonyl)-4-(p-tolylethynyl)benzamide; or a pharmaceutically acceptable salt thereof.

18. A compound selected from the group consisting of:
4-(Benzyloxy)-3-(cyclopropylmethoxy)-N-(2-sulfamoylphenylsulfonyl)benzamide;
3-Methoxy-N-(2-sulfamoylphenylsulfonyl)-4-(p-tolyloxymethyl)benzamide;
3-(2,2-Difluoroethoxy)-4-(phenylethynyl)-N-(2-sulfamoylphenylsulfonyl)benzamide;
3-(2,2-Difluoroethoxy)-N-(2-sulfamoylphenylsulfonyl)-4-(p-tolylethynyl)benzamide;
4-(Cyclopentylethynyl)-3-(2,2-difluoroethoxy)-N-(2-sulfamoylphenylsulfonyl)benzamide;
3-(2,2-Difluoroethoxy)-4-(3,3-dimethylbut-1-ynyl)-N-(2-sulfamoylphenylsulfonyl)-benzamide;
4-(Benzofuran-2-yl)-3-(2,2-difluoroethoxy)-N-(2-sulfamoylphenylsulfonyl)benzamide;
N-(2-Sulfamoylphenylsulfonyl)-4-(o-tolylethynyl)benzamide;
N-(2-sulfamoylphenylsulfonyl)-4-(p-tolylethynyl)benzamide;
4-((4-Chlorophenyl)ethynyl)-N-(2-sulfamoylphenylsulfonyl)benzamide;
4-((4-Fluorophenyl)ethynyl)-N-(2-sulfamoylphenylsulfonyl)benzamide;
4-((3-Chlorophenyl)ethynyl)-N-(2-sulfamoylphenylsulfonyl)benzamide;
4-((3-Fluorophenyl)ethynyl)-N-(2-sulfamoylphenylsulfonyl)benzamide;
3-Methoxy-N-(2-sulfamoylphenylsulfonyl)-4-(o-tolylethynyl)benzamide;
4-((4-Chlorophenyl)ethynyl)-3-methoxy-N-(2-sulfamoylphenylsulfonyl)benzamide;
4-((4-Fluorophenyl)ethynyl)-3-methoxy-N-(2-sulfamoylphenylsulfonyl)benzamide;
3-Methoxy-N-(2-sulfamoylphenylsulfonyl)-4-(p-tolylethynyl)benzamide;
3-Methoxy-N-(2-sulfamoylphenylsulfonyl)-4-(m-tolylethynyl)benzamide;
4-((3-Chlorophenyl)ethynyl)-3-methoxy-N-(2-sulfamoylphenylsulfonyl)benzamide;
4-((3-Fluorophenyl)ethynyl)-3-methoxy-N-(2-sulfamoylphenylsulfonyl)benzamide;
4-(Benzofuran-2-yl)-3-(cyclopropylmethoxy)-N-(2-sulfamoylphenylsulfonyl)benzamide;
3-(Cyclopropylmethoxy)-4-(3,3-dimethylbut-1-ynyl)-N-(2-sulfamoylphenylsulfonyl)-benzamide;
3-(Cyclopropylmethoxy)-4-(3-methylbut-1-ynyl)-N-(2-sulfamoylphenylsulfonyl)-benzamide;
N-(2-Sulfamoylphenylsulfonyl)-4-((3,3,4,4-tetrafluoropyrrolidin-1-yl)methyl)benzamide;
3-(3-Hydroxy-3-methylbutoxy)-4-(3-methylbut-1-ynyl)-N-(2-sulfamoylphenylsulfonyl)-benzamide;
3-Isopropoxy-4-(3-methylbut-1-ynyl)-N-(2-sulfamoylphenylsulfonyl)benzamide;
4-(Cyclopropylethynyl)-3-(3-methoxy-3-methylbutoxy)-N-(2-sulfamoylphenylsulfonyl)-benzamide;
3-Isobutoxy-4-(3-methylbut-1-ynyl)-N-(2-sulfamoylphenylsulfonyl)benzamide;
3-Methoxy-4-(4-methylbenzyloxy)-N-(2-sulfamoylphenylsulfonyl)benzamide;
4-(Benzyloxy)-N-(2-sulfamoylphenylsulfonyl)-3-(3,3,3-trifluoropropoxy)benzamide;
4-(3,3-Dicyclopropyl-3-hydroxyprop-1-ynyl)-N-(2-sulfamoylphenylsulfonyl)-benzamide;
3-(Hydroxymethyl)-4-(3-methylhex-1-ynyl)-N-(2-sulfamoylphenylsulfonyl)benzamide;
3-Isopropoxy-N-(2-sulfamoylphenylsulfonyl)-4-((4-(trifluoromethyl)phenyl)ethynyl)-benzamide;
4-(3-Methylhex-1-ynyl)-N-(2-sulfamoylphenylsulfonyl)-3-(2-(2,2,2-trifluoroethoxy)-ethoxy)benzamide;
4-(3-Methylhex-1-ynyl)-N-(2-sulfamoylphenylsulfonyl)-3-(3,3,3-trifluoropropoxy)benzamide;
4-(3,3-Dicyclopropylprop-1-ynyl)-N-(2-sulfamoylphenylsulfonyl)-3-(3,3,3-trifluoropropoxy)benzamide;
3-Methoxy-4-(3-methylhex-1-ynyl)-N-(2-sulfamoylphenylsulfonyl)benzamide;
3-Methoxy-N-(2-sulfamoylphenylsulfonyl)-4-((4-(trifluoromethyl)phenyl)ethynyl)-benzamide;
3-(Hydroxymethyl)-N-(2-sulfamoylphenylsulfonyl)-4-((4-(trifluoromethyl)phenyl)-ethynyl)benzamide;
4-(3-Methylhex-1-ynyl)-N-(2-sulfamoylphenylsulfonyl)benzamide;
N-(2-Sulfamoylphenylsulfonyl)-4-((4-(trifluoromethyl)phenyl)ethynyl)benzamide;
4-(6-Chlorohex-1-ynyl)-N-(2-sulfamoylphenylsulfonyl)benzamide;
N-(2-Sulfamoylphenylsulfonyl)-6-((4-(trifluoromethyl)phenyl)ethynyl)nicotinamide;
5-Chloro-6-(3-methylbut-1-ynyl)-N-(2-sulfamoylphenylsulfonyl)nicotinamide;
4-(3-Methylbut-1-ynyl)-N-(2-sulfamoylphenylsulfonyl)-3-(3,3,3-trifluoropropoxy)-benzamide;
5-Fluoro-6-(3-methylbut-1-ynyl)-N-(2-sulfamoylphenylsulfonyl)nicotinamide;
5-Fluoro-N-(2-sulfamoylphenylsulfonyl)-6-((4-(trifluoromethyl)phenyl)ethynyl-nicotinamide;
N-(2-Sulfamoylphenylsulfonyl)-6-((4-(trifluoromethyl)phenyl)ethynyl)-5-(3,3,3-trifluoro-propoxy)nicotinamide;
6-(3-Methylbut-1-ynyl)-N-(2-sulfamoylphenylsulfonyl)-5-(3,3,3-trifluoropropoxy)-nicotinamide;
3-Methyl-4-(3-methylbut-1-ynyl)-N-(2-sulfamoylphenylsulfonyl)benzamide;
4-(3-Methylbut-1-ynyl)-N-(2-sulfamoylphenylsulfonyl)-3-(2-(2,2,2-trifluoroethoxy)-ethoxy)benzamide;
3-(2,2-Difluoroethoxy)-4-(3-methylbut-1-ynyl)-N-(2-sulfamoylphenylsulfonyl)-benzamide;
4-(3-Methylbut-1-ynyl)-N-(2-sulfamoylphenylsulfonyl)-3-(2,2,2-trifluoroethoxy)-benzamide;
4-(3-Methylbut-1-ynyl)-N-(2-sulfamoylphenylsulfonyl)-3-(trifluoromethoxy)-benzamide;
4-(3-Methylbut-1-ynyl)-N-(2-sulfamoylphenylsulfonyl)-3-((3,3,3-trifluoropropoxy)-methyl)benzamide;
4-(3-Methylbut-1-ynyl)-N-(2-sulfamoylphenylsulfonyl)-3-((2,2,2-trifluoroethoxy)methyl)-benzamide;
4-(Benzofuran-2-yl)-3-fluoro-N-(2-sulfamoylphenylsulfonyl)benzamide;
4-(3-Methylbut-1-ynyl)-N-(2-sulfamoylphenylsulfonyl)-3-(2,2,3,3-tetrafluoropropoxy)-benzamide;
4-(Benzofuran-2-yl)-N-(2-sulfamoylphenylsulfonyl)-3-(3,3,3-trifluoropropoxy)benzamide;
4-(3,3-Dimethylbut-1-ynyl)-N-(2-sulfamoylphenylsulfonyl)-3-(3,3,3-trifluoropropoxy)-benzamide;
4-(3,3-Dimethylbut-1-ynyl)-N-(2-sulfamoylphenylsulfonyl)-3-(2-(2,2,2-trifluoroethoxy)-ethoxy)benzamide;
4-(Benzofuran-2-yl)-N-(2-sulfamoylphenylsulfonyl)-3-(2-(2,2,2-trifluoroethoxy)ethoxy)-benzamide;
4-(Phenylethynyl)-N-(2-sulfamoylphenylsulfonyl)-3-(2-(2,2,2-trifluoroethoxy)ethoxy)-benzamide;
4-(3-Methylbut-1-ynyl)-N-(2-sulfamoylphenylsulfonyl)-3-(4,4,4-trifluorobutoxy)-benzamide;
4-(Phenylethynyl)-N-(2-sulfamoylphenylsulfonyl)-3-(3,3,3-trifluoropropoxy)benzamide;
4-(Cyclopentylethynyl)-N-(2-sulfamoylphenylsulfonyl)-3-(3,3,3-trifluoropropoxy)-benzamide;
4-(Cyclopentylethynyl)-N-(2-sulfamoylphenylsulfonyl)-3-(2-(2,2,2-trifluoroethoxy)-ethoxy)benzamide;
4-(Cyclopentylethynyl)-3-(3,3-difluoropropoxy)-N-(2-sulfamoylphenylsulfonyl)-benzamide;
4-(Cyclopentylethynyl)-N-(2-sulfamoylphenylsulfonyl)-3-(4,4,4-trifluorobutoxy)-benzamide;
3-(3,3-Difluoropropoxy)-N-(2-sulfamoylphenylsulfonyl)-4-(p-tolylethynyl)benzamide;
3-(3,3-Difluoropropoxy)-4-(3-methylbut-1-ynyl)-N-(2-sulfamoylphenylsulfonyl)-benzamide;
3-(3-Fluoropropoxy)-4-(3-methylbut-1-ynyl)-N-(2-sulfamoylphenylsulfonyl)benzamide;
3-Methoxy-4-(phenylethynyl)-N-(2-sulfamoylphenylsulfonyl)benzamide;
3-Methoxy-4-(3-methylbut-1-ynyl)-N-(2-sulfamoylphenylsulfonyl)benzamide;
4-Hydroxy-benzene-1,2-disulfonic acid 2-amide 1-(4-benzofuran-2-yl-benzoylamide);
4-Methoxy-benzene-1,2-disulfonic acid 2-amide 1-(4-benzofuran-2-yl-benzoylamide);
4-Hydroxy-benzene-1,2-disulfonic acid 2-amide 1-[4-(3,3-dimethyl-but-1-ynyl)-benzoylamide];
4-Methoxy-benzene-1,2-disulfonic acid 2-amide 1-[4-(3,3-dimethyl-but-1-ynyl)-benzoylamide] and
3-Methoxy-N-(2-sulfamoylphenylsulfonyl)-4-(p-tolylethynyl)benzamide; or a pharmaceutically acceptable salt thereof.

19. The compound N-(2-sulfamoylphenylsulfonyl)-4-(p-tolylethynyl)benzamide or a pharmaceutically acceptable salt thereof.

20. The compound 4-(Benzofuran-2-yl)-3-(cyclopropylmethoxy)-N-(2-sulfamoylphenylsulfonyl)benzamide or a pharmaceutically acceptable salt thereof.

21. The compound N-(2-Sulfamoylphenylsulfonyl)-4-((4-(trifluoromethyl)phenyl)ethynyl)benzamide or a pharmaceutically acceptable salt thereof.

22. A pharmaceutical composition comprising a compound or a pharmaceutically acceptable salt thereof according to claim 16 in association with a pharmaceutically acceptable adjuvant, diluent or carrier.

23. A process for the preparation of a pharmaceutical composition according to claim 22 which comprises mixing a compound or a pharmaceutically acceptable salt thereof according to claim 16 with a pharmaceutically acceptable adjuvant, diluent or carrier.

24. A method of treating, or reducing the risk of, an inflammatory disease or condition which comprises administering to a patient in need thereof a therapeutically effective amount of a compound or a pharmaceutically acceptable salt thereof according to claim 16.

25. A method of treating, or reducing the risk of, acute or chronic pain, nociceptive pain or neuropathic pain which comprises administering to a patient in need thereof a therapeutically effective amount of a compound or a pharmaceutically acceptable salt thereof according to claim 16.

26. A method of treating, or reducing the risk of, osteoarthritis, rheumatoid arthritis, benign or malignant neoplasias, apnea, sudden infant death (SID), atherosclerosis, cancer, aneurysm, stroke, hyperthermia, myositis, Alzheimer's disease or arthritis which comprises administering to a patient in need thereof a therapeutically effective amount of a compound or a pharmaceutically acceptable salt thereof according to claim 16.
